(19) United States
(12) Patent Application Publication
(10) Pub. No.: US 2024/0228483 A1
(43) Pub. Date: Jul. 11, 2024

(54) ORGANIC COMPOUND, ORGANIC LIGHT EMITTING DIODE AND ORGANIC LIGHT EMITTING DEVICE HAVING THE COMPOUND

(71) Applicant: LG DISPLAY CO., LTD., Seoul (KR)

(72) Inventor: Ji-Ae LEE, Paju-si (KR)

(73) Assignee: LG DISPLAY CO., LTD., Seoul (KR)

(21) Appl. No.: 18/385,217

(22) Filed: Oct. 30, 2023

(30) Foreign Application Priority Data
Dec. 16, 2022 (KR) 10-2022-0176582

(51) Int. Cl. C07D 471/04; C09K 11/06; H10K 50/13; H10K 85/60

(52) U.S. Cl. CPC C07D 471/04; C09K 11/06; H10K 50/13; H10K 85/6572; C09K 2211/1018

(57) ABSTRACT

The present disclosure relates to an organic compound having a wide plate-like structure fused with multiple aromatic and/or hetero aromatic rings, an organic light emitting diode and an organic light emitting device where an emissive layer comprises the organic compound. The organic compound has a structure of Chemical Formula 1.

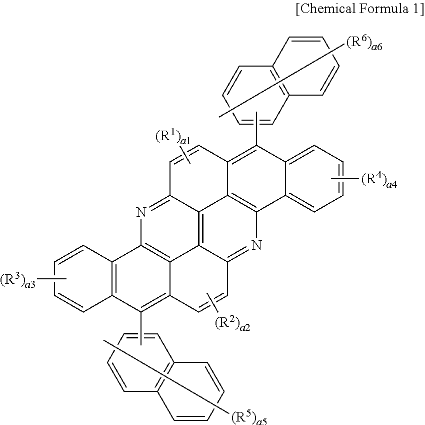

### CROSS-REFERENCE TO RELATED APPLICATIONS

This application claims the benefit of and the priority of Korean Patent Application No. 10-2022-0176582, filed in the Republic of Korea on Dec. 16, 2022, which is expressly incorporated hereby in its entirety into the present application.

### TECHNICAL FIELD

The present disclosure relates to an organic compound, and more particularly to, an organic compound with beneficial luminous efficiency and luminous lifespan and an organic light emitting diode and an organic light emitting device including the organic compound.

### BACKGROUND ART

A flat display device including an organic light emitting diode (OLED) has attracted attention as a display device that can replace a liquid crystal display device (LCD). The electrode configurations in the OLED can implement unidirectional or bidirectional images. Also, the OLED can be formed even on a flexible transparent substrate such as a plastic substrate so that a flexible or a foldable display device can be realized with ease using the OLED. In addition, the OLED can be driven at a lower voltage and the OLED has advantageous high color purity compared to the LCD.

Since fluorescent material uses only singlet excitons in the luminous process, the related art fluorescent material shows low luminous efficiency. On the contrary, phosphorescent material can show high luminous efficiency since it uses triplet exciton as well as singlet excitons in the luminous process. However, examples of phosphorescent material comprise metal complexes, which have a short luminous lifespan for commercial use. It is necessary to develop a compound or an organic light emitting diode having improved luminous efficiency and luminous lifespan.

### SUMMARY

Accordingly, some embodiments of the present disclosure are directed to an organic light emitting diode and an organic light emitting device that substantially obviate one or more of the problems due to the limitations and disadvantages of the related art.

An aspect of the present disclosure is to provide an organic compound with beneficial luminous efficiency and luminous lifespan, as well as an organic light emitting diode and an organic light emitting device having the organic compound.

Additional features and aspects will be set forth in the description that follows, and in part will be apparent from the description, or may be learned by practice of the disclosed concepts provided herein. Other features and aspects of the disclosed concept may be realized and attained by the structure particularly pointed out in the written description, or derivable therefrom, and the claims hereof as well as the appended drawings.

To achieve these and other aspects of the inventive concepts, as embodied and broadly described, in one aspect, the present disclosure provides an organic compound having the following structure of Chemical Formula 1:

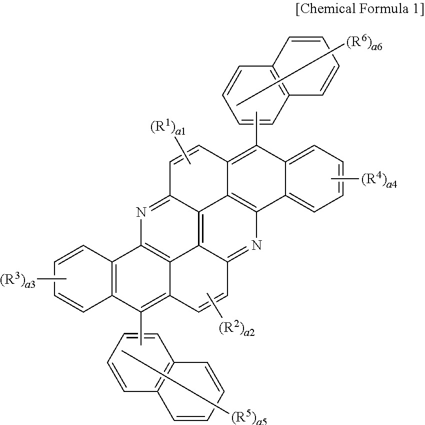

wherein, in Chemical Formula 1,
each of R¹, R², R³, R⁴, R⁵ and R⁶ is independently a halogen atom, a cyano group, an unsubstituted or substituted C₁-C₂₀ alkyl group, an unsubstituted or substituted C₁-C₂₀ alkyl amino group, an unsubstituted or substituted C₆-C₃₀ aryl group, an unsubstituted or substituted C₃-C₃₀ hetero aryl group, an unsubstituted or substituted C₆-C₃₀ aryl amino group or an unsubstituted or substituted C₃-C₃₀ hetero aryl amino group, where each R¹ is identical to or different from each other when a1 is 2, each R² is identical to or different from each other when a2 is 2, each R³ is identical to or different from each other when a3 is 2, 3 or 4, each R⁴ is identical to or different from each other when a4 is 2, 3 or 4, each R⁵ is identical to or different from each other when a5 is 2, 3, 4, 5, 6 or 7 and each R⁶ is identical to or different from each other when a6 is 2, 3, 4, 5, 6 or 7;
each of a1 and a2 is independently 0, 1 or 2;
each of a3 and a4 is independently 0, 1, 2, 3 or 4; and
each of a5 and a6 is independently 0, 1, 2, 3, 4, 5, 6 or 7.

The organic compound can have the following structure of Chemical Formula 2A or Chemical Formula 2B:

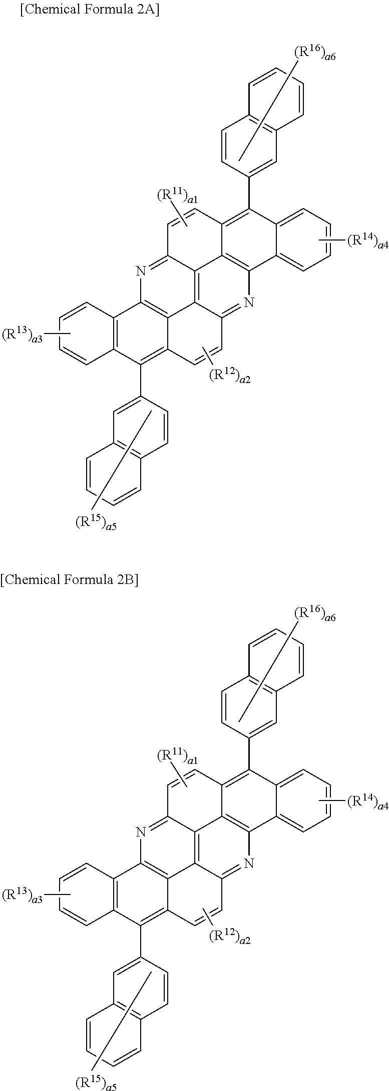

wherein, in Chemical Formulae 2A and 2B,
each of a1, a2, a3, a4, a5 and a6 is identical as defined in Chemical Formula 1,
each of R¹¹, R¹², R¹³, R¹⁴, R¹⁵ and R¹⁶ is independently an unsubstituted or substituted C₁-C₂₀ alkyl group or an unsubstituted or C₁-C₁₀ alkyl-substituted C₆-C₃₀ aryl group, where each R¹¹ is identical to or different from each other when a1 is 2, each R¹² is identical to or different from each other when a2 is 2, each R¹³ is identical to or different from each other when a3 is 2, 3 or 4, each R¹⁴ is identical to or different from each other when a4 is 2, 3 or 4, each R¹⁵ is identical to or different from each other when a5 is 2, 3, 4, 5, 6 or 7 and each R¹⁶ is identical to or different from each other when a6 is 2, 3, 4, 5, 6 or 7.

As an example, each of R¹, R², R³, R⁴, R⁵ and R⁶ can be independently an unsubstituted or substituted C₁-C₁₀ alkyl group or an unsubstituted or C₁-C₁₀ alkyl-substituted C₆-C₃₀ aryl group, each of a1 and a2 can be 0, and each of a3, a4, a5 and a6 can be independently 0 or 1.

In another embodiment, each of a1 and a2 can be 0, each of R³ and R⁴ can be independently an unsubstituted or C₁-C₁₀ alkyl-substituted C₆-C₃₀ aryl group, each of a3 and a4 can be independently 0 or 1, each of R⁵ and R⁶ can be independently an unsubstituted or substituted C₁-C₁₀ alkyl group, and each of a5 and a6 can be independently 0 or 1.

In another aspect, the present disclosure provides an organic light emitting diode that comprises a first electrode; a second electrode facing the first electrode; and an emissive layer disposed between the first electrode and the second electrode, wherein the emissive layer comprises the above organic compound.

The emissive layer can comprise at least one emitting material layer, and the at least one emitting material layer can comprise the organic compound.

The at least one emitting material layer can comprise an emitter, and the emitter can comprise the organic compound.

The at least one emitting material layer can further comprise a first host.

The first host can comprise a compound of Chemical Formula 4:

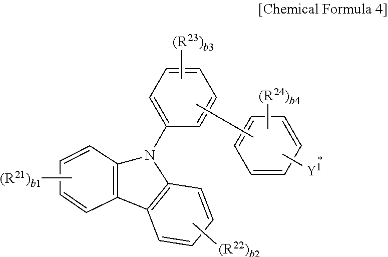

wherein, in Chemical Formula 4,
each of R²¹ and R²² is independently an unsubstituted or substituted C₁-C₂₀ alkyl group or an unsubstituted or substituted C₆-C₃₀ aryl group, where each R²¹ is identical to or different from each other when b1 is 2, 3 or 4, and each R²² is identical to or different from each other when b2 is 2, 3 or 4;
each of R²³ and R²⁴ is independently an unsubstituted or substituted C₁-C₂₀ alkyl group or an unsubstituted or substituted C₆-C₃₀ aryl group, where each R²³ is identical to or different from each other when b3 is 2, 3 or 4, and each R²⁴ is identical to or different from each other when b4 is 2, 3 or 4, or optionally,
optionally, R²³ and R²⁴ may be further linked together to form an unsubstituted or substituted hetero ring;
Y¹ has the following structure of Chemical Formula 5A or Chemical Formula 5B;
each of b1, b2, b3 and b4 is independently 0, 1, 2, 3 or 4; and
the asterisk indicates a link to the following Chemical Formula 5A or Chemical Formula 5B:

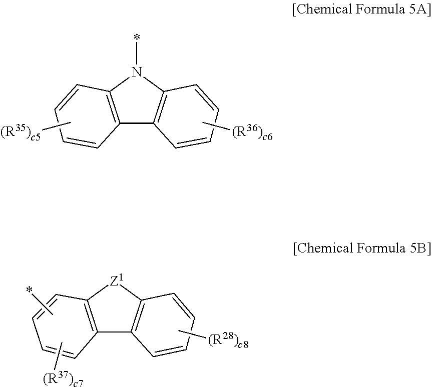

wherein, in Chemical Formulae 5A and 5B,
each of R³⁵, R³⁶, R³⁷ and R³⁸ is independently an unsubstituted or substituted C₁-C₂₀ alkyl group or an unsubstituted or substituted C₆-C₃₀ aryl group, where each R³⁵ is identical to or different from each other when c5 is 2, 3 or 4, each R³⁶ is identical to or different from each other when c6 is 2, 3 or 4, each R³⁷ is identical to or different from each other when c7 is 2 or 3, and each R³⁸ is identical to or different from each other when c8 is 2, 3 or 4;
Z¹ is NR³⁹, O or S, where R³⁹ is hydrogen, an unsubstituted or substituted C₁-C₂₀ alkyl group or an unsubstituted or substituted C₆-C₃₀ aryl group;
each of c5, c6 and c8 is independently 0, 1, 2, 3 or 4;
c7 is 0, 1, 2 or 3; and
asterisk indicates a link position.

The at least one emitting material layer can further comprise a second host.

In some embodiments, the emissive layer can have a single emitting unit, or multiple emitting parts to form a tandem structure.

In another aspect, the present disclosure provides an organic light emitting device, for example, an organic light emitting display device or organic light emitting luminance device, where the organic light emitting diode is disposed on a substrate.

The organic compound may have a fused structure of multiple aromatic and/or hetero aromatic rings to have a wide plate-like structure. The emitting material layer may comprise at least one host. The host can transfer exciton energy to the organic compound by FRET mechanism where the singlet exciton of the host is transferred to the singlet exciton of the organic compound of the emitter.

The organic compound can utilize only the singlet exciton because the organic compound may be fluorescent material. The amount of the singlet exciton, which can be utilized by the organic compound and can contribute the emission of the organic compound, is increased as the exciton energy is transferred to the organic compound by FRET mechanism that can transfer singlet-singlet exciton energy. Accordingly, the luminous efficiency and the luminous lifespan of an organic light emitting diode can be improved by using the inventive compound as final emitting material.

It is to be understood that both the foregoing general description and the following detailed description are exemplary and explanatory, and are intended to provide further explanation of the inventive concepts as claimed.

### BRIEF DESCRIPTION OF THE DRAWINGS

The accompanying drawings, which are comprised to provide a further understanding of the disclosure, are incorporated in and constitute a part of this application, illustrate embodiments of the disclosure and together with the description serve to explain principles of the disclosure.

FIG. 1 illustrates a schematic circuit diagram of an organic light emitting display device in accordance with the present disclosure.

FIG. 2 illustrates a schematic cross-sectional view of an organic light emitting display device as an example of an organic light emitting device in accordance with an example embodiment of the present disclosure.

FIG. 3 illustrates a schematic cross-sectional view of an organic light emitting diode having a single emitting part in accordance with an example embodiment of the present disclosure.

FIG. 4 illustrates a schematic cross-sectional view of an organic light emitting display device in accordance with another example embodiment of the present disclosure.

FIG. 5 illustrates a schematic cross-sectional view of an organic light emitting diode with two emitting parts forming a tandem structure in accordance with another example embodiment of the present disclosure.

FIG. 6 illustrates a schematic cross-sectional view of an organic light emitting diode with three emitting parts forming a tandem structure in accordance with another example embodiment of the present disclosure.

### DETAILED DESCRIPTION

Reference will now be made in detail to aspects of the disclosure, examples of which are illustrated in the accompanying drawings. Wherever possible, the same reference numbers will be used throughout the drawings to refer to the same or like parts.

### [Organic Compound]

The organic compound of the present disclosure may have a wide plate-like structure to receive efficiently singlet exciton energy from a host or other luminous materials. The luminous efficiency and the luminous lifespan of an organic light emitting diode can be improved by utilizing the organic compound as a final emitter. The organic compound can have the following structure of Chemical Formula 1:

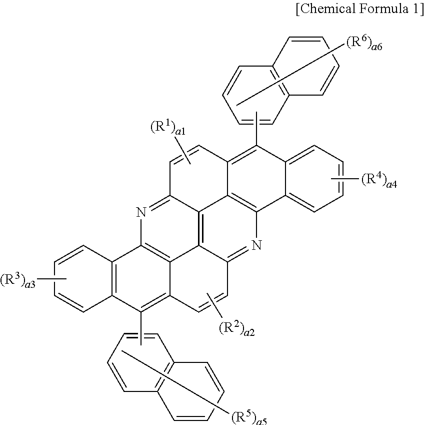

wherein, in Chemical Formula 1,
each of R¹, R², R³, R⁴, R⁵ and R⁶ is independently a halogen atom, a cyano group, an unsubstituted or substituted C₁-C₂₀ alkyl group, an unsubstituted or substituted C₁-C₂₀ alkyl amino group, an unsubstituted or substituted C₆-C₃₀ aryl group, an unsubstituted or substituted C₃-C₃₀ hetero aryl group, an unsubstituted or substituted C₆-C₃₀ aryl amino group or an unsubstituted or substituted C₃-C₃₀ hetero aryl amino group, where each R¹ is identical to or different from each other when a1 is 2, each R² is identical to or different from each other when a2 is 2, each R³ is identical to or different from each other when a3 is 2, 3 or 4, each R⁴ is identical to or different from each other when a4 is 2, 3 or 4, each R⁵ is identical to or different from each other when a5 is 2, 3, 4, 5, 6 or 7 and each R⁶ is identical to or different from each other when a6 is 2, 3, 4, 5, 6 or 7;
each of a1 and a2 is independently 0, 1 or 2;
each of a3 and a4 is independently 0, 1, 2, 3 or 4; and
each of a5 and a6 is independently 0, 1, 2, 3, 4, 5, 6 or 7.

As used herein, the term “unsubstituted” means that hydrogen is directly linked to a carbon atom. “Hydrogen,” as used herein, can refer to protium, deuterium and tritium.

As used herein, “substituted” means that the hydrogen is replaced with a substituent. The substituent can comprise, but is not limited to, an unsubstituted or substituted C₁-C₂₀ alkyl group, an unsubstituted or substituted C₁-C₂₀ alkoxy, halogen, a cyano group, a hydroxyl group, a carboxylic group, a carbonyl group, an amino group, a C₁-C₁₀ alkyl amino group, a C₆-C₃₀ aryl amino group, a C₃-C₃₀ hetero aryl amino group, a nitro group, a hydrazyl group, a sulfonate group, an unsubstituted or substituted C₁-C₁₀ alkyl silyl group, an unsubstituted or d substituted C₁-C₁₀ alkoxy silyl group, an unsubstituted or substituted C₃-C₂₀ cyclo alkyl silyl group, an unsubstituted or substituted C₆-C₃₀ aryl silyl group, an unsubstituted or substituted C₆-C₃₀ aryl group, an unsubstituted or substituted C₃-C₃₀ hetero aryl group, and a group formed by any combination of these groups.

For example, each of the C₆-C₃₀ aryl group and the C₃-C₃₀ hetero aryl group can be substituted with at least one of C₁-C₂₀ alkyl, C₆-C₃₀ aryl and C₃-C₃₀ hetero aryl.

As used herein, the term “hetero” in terms such as “a hetero aromatic group,” “a hetero cyclo alkylene group,” “a hetero arylene group,” “a hetero aryl alkylene group,” “a hetero aryl oxylene group,” “a hetero cyclo alkyl group,” “a hetero aryl group,” “a hetero aryl alkyl group,” “a hetero aryloxy group,” “a hetero aryl amino group” and the likes means that at least one carbon atom, for example 1 to 5 carbons atoms, constituting an aliphatic chain, an alicyclic group or ring or an aromatic group or ring is substituted with at least one hetero atom selected from the group consisting of N, O, S and P.

As used herein, the C₁-C₂₀ alkyl group can comprise, but is not limited to, a straight or branched alkyl chain alkyl group having 1 to 20 carbon atoms, 1 to 15 carbon atoms, 1 to 10 carbon atoms, 1 to 4 carbon atoms, such as a methyl group, an ethyl group, a propyl group, an isopropyl group, a butyl group, an isobutyl group, a sec-butyl group, a tert-butyl group, a pentyl group, a tert-pentyl group, a neopentyl group, isopentyl group, a hexyl group, a heptyl group, a octyl group, a nonyl group, a decyl group, a undecyl group, a dodecyl group, a tridecyl group, a tetradecyl group, a pentadecyl group, a hexadecyl group, a octadecyl group, a nonadecyl group, and a icosanyl group and the like.

As used herein, the C₆-C₃₀ aryl group can comprise, but is not limited to, an unfused or fused aryl group having 6 to 30 carbon atoms, 6 to 20 carbon atoms, 6 to 10 carbon atoms such as phenyl, biphenyl, terphenyl, naphthyl, anthracenyl, pentalenyl, indenyl, indeno-indenyl, heptalenyl, biphenylenyl, indacenyl, phenalenyl, phenanthrenyl, benzo-phenanthrenyl, dibenzo-phenanthrenyl, azulenyl, pyrenyl, fluoranthenyl, triphenylenyl, chrysenyl, tetraphenylenyl, tetracenyl, pleiadenyl, picenyl, pentaphenylenyl, pentacenyl, fluorenyl, indeno-fluorenyl or spiro-fluorenyl. The C₆-C₃₀ arylene group can comprise, but is not limited to, any bivalent linking group corresponding to the above aryl group. As used herein, the C₆-C₃₀ arylene group can be a bivalent linking group corresponding to each of the C₆-C₃₀ aryl group.

As used herein, the C₃-C₃₀ hetero aryl group can comprise, but is not limited to, an unfused or fused hetero aryl group having 3 to 30 carbon atoms, 3 to 10 carbon atoms, 3 to 6 carbon atoms, such as pyrrolyl, pyridinyl, pyrimidinyl, pyrazinyl, pyridazinyl, triazinyl, tetrazinyl, imidazolyl, pyrazolyl, indolyl, iso-indolyl, indazolyl, indolizinyl, pyrrolizinyl, carbazolyl, benzo-carbazolyl, dibenzo-carbazolyl, indolo-carbazolyl, indeno-carbazolyl, benzo-furo-carbazolyl, benzo-thieno-carbazolyl, carbolinyl, quinolinyl, iso-quinolinyl, phthlazinyl, quinoxalinyl, cinnolinyl, quinazolinyl, quinolizinyl, purinyl, benzo-quinolinyl, benzo-iso-quinolinyl, benzo-quinazolinyl, benzo-quinoxalinyl, acridinyl, phenazinyl, phenoxazinyl, phenothiazinyl, phenanthrolinyl, perimidinyl, phenanthridinyl, pteridinyl, naphthyridinyl, furanyl, pyranyl, oxazinyl, oxazolyl, oxadiazolyl, triazolyl, dioxinyl, benzo-furanyl, dibenzo-furanyl, thiopyranyl, xanthenyl, chromenyl, iso-chromenyl, thioazinyl, thiophenyl, benzo-thiophenyl, dibenzo-thiophenyl, difuro-pyrazinyl, benzofuro-dibenzo-furanyl, benzothieno-benzo-thiophenyl, benzothieno-dibenzo-thiophenyl, benzothieno-benzo-furanyl, benzothieno-dibenzo-furanyl, xanthene-linked spiro acridinyl, dihydroacridinyl substituted with at least one C₁-C₁₀ alkyl and N-substituted spiro fluorenyl. The C₃-C₃₀ hetero arylene group can comprise, but is not limited to, any bivalent linking group corresponding to the above hetero aryl group.

As used herein, the term “halogen” refers to bromo, chloro, fluoro or iodo.

As an example, each of the aryl group or the hetero aryl group of R¹ to R⁶ in Chemical Formula 1 can consist of one to four, for example, one to three aromatic and/or hetero aromatic rings. When the number of the aromatic and/or hetero aromatic rings of R¹ to R⁶ becomes more than four, conjugated structure among the within the entire molecule becomes too long, thus, the organometallic compound can have too narrow energy bandgap. For example, each of the aryl group or the hetero aryl group of R¹ to R⁶ can comprise independently, but is not limited to, phenyl, biphenyl, naphthyl, anthracenyl, pyrrolyl, triazinyl, imidazolyl, pyrazolyl, pyridinyl, pyrazinyl, pyrimidinyl, pyridazinyl, furanyl, benzo-furanyl, dibenzo-furanyl, thiophenyl, benzo-thiophenyl, dibenzo-thiophenyl, carbazolyl, acridinyl, carbolinyl, phenazinyl, phenoxazinyl, or phenothiazinyl.

The organic compound having the structure of Chemical Formula 1 may comprise a fused ring system of multiple aromatic rings and/or hetero aromatic rings, so that the organic compound has a wide plate-like structure. The excited singlet exciton energy of the host and/or other luminous materials can be transferred efficiently to the singlet exciton of the organic compound having the structure of Chemical Formula 1 through Forster Resonance Energy Transfer (FRET) mechanism.

The organic compound may not be able to utilize triplet excitons because the organic compound having the structure of Chemical Formula 1 may be fluorescent material. Only the singlet exciton energy transferred by the FRET mechanism can contribute to the emission of the organic compound having the structure of Chemical Formula 1. The amount of singlet exciton energy that can be utilized by the organic compound having the structure of Chemical Formula 1 and be contributed the emission of the organic compound is increased as the exciton energies may be transferred to the organic compound through the FRET mechanism that can transfer only singlet-singlet exciton energy that can contribute the emission of the organic compound having the structure of Chemical Formula 1. The luminous efficiency and luminous lifespan of an organic light emitting diode can be improved by using the organic compound having the structure of Chemical Formula 1 as the final emitting material.

For example, the organic compound having the structure of Chemical Formula 1 can emit red to yellow-green color light. Applying the organic compound into an emissive layer between two electrodes enables the organic light emitting diode to improve its luminous efficiency and luminous lifespan.

In some embodiments, each of R¹, R², R³, R⁴, R⁵ and R⁶ in Chemical Formula 1 can be independently an unsubstituted or substituted C₁-C₂₀ alkyl group or an unsubstituted or C₁-C₁₀ alkyl-substituted C₆-C₃₀ aryl group. The organic compound with such a structure can comprise an organic compound having the following structure of Chemical Formula 2A or Chemical Formula 2B:

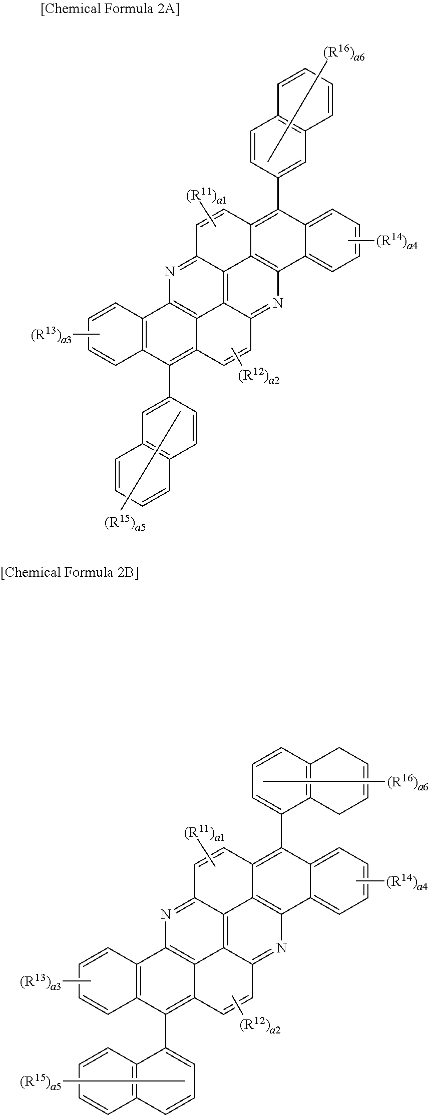

wherein, in Chemical Formulae 2A and 2B,
each of a1, a2, a3, a4, a5 and a6 is identical as defined in Chemical Formula 1,
each of R¹¹, R¹², R¹³, R¹⁴, R¹⁵ and R¹⁶ is independently an unsubstituted or substituted C₁-C₂₀ alkyl group or an unsubstituted or C₁-C₁₀ alkyl-substituted C₆-C₃₀ aryl group, where each R¹¹ is identical to or different from each other when a1 is 2, each R¹² is identical to or different from each other when a2 is 2, each R¹³ is identical to or different from each other when a3 is 2, 3 or 4, each R¹⁴ is identical to or different from each other when a4 is 2, 3 or 4, each R¹⁵ is identical to or different from each other when a5 is 2, 3, 4, 5, 6 or 7 and each R¹⁶ is identical to or different from each other when a6 is 2, 3, 4, 5, 6 or 7.

In some embodiments, each of R¹, R², R³, R⁴, R⁵ and R⁶ in Chemical Formula 1 can be independently a C₁-C₁₀ alkyl group (e.g., methyl, ethyl, n- or iso-propyl or tert-butyl) or an unsubstituted or a C₁-C₁₀ alkyl (e.g., methyl, ethyl, n- or iso-propyl or tert-butyl)-substituted C₆-C₃₀ aryl group (e.g., phenyl or naphthyl), each of a1 and a2 can be 0, and each of a3, a4, a5 and a6 can be independently 0 or 1, but is not limited thereto.

In another embodiments, each of a1 and a2 can be 0, each of R³ and R⁴ can be independently an unsubstituted or C₁-C₁₀ alkyl (e.g., methyl, ethyl, n- or iso-propyl or tert-butyl)-substituted C₆-C₃₀ aryl group (e.g., phenyl or naphthyl), each of a3 and a4 can be independently 0 or 1, each of R⁵ and R⁶ can be independently a C₁-C₁₀ alkyl group (e.g., methyl, ethyl, n- or iso-propyl or tert-butyl), and each of a5 and a6 can be independently 0 or 1.

For example, the organic compound having the structure of Chemical Formula 1 can be, but is not limited to, at least one of the following compounds of Chemical Formula 3 (compounds 1-1 to 1-56):

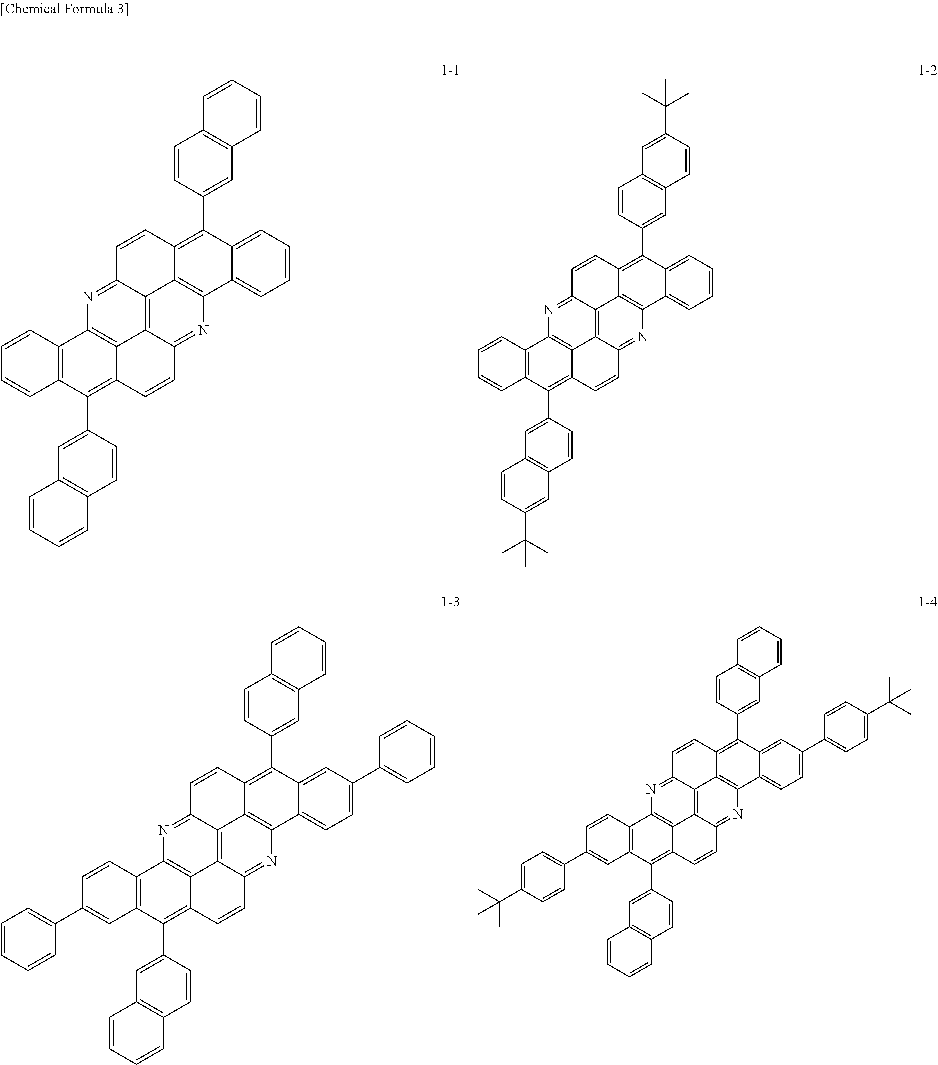 [SMILES: c1ccc2cc(-c3c4ccccc4c4nc5ccc6c(-c7ccc8ccccc8c7)c7ccccc7c7nc8ccc3c4c8c5c67)ccc2c1 ; CC(C)(C)c1ccc2cc(-c3c4ccccc4c4nc5ccc6c(-c7ccc8cc(C(C)(C)C)ccc8c7)c7ccccc7c7nc8ccc3c4c8c5c67)ccc2c1 ; c1ccc(-c2ccc3c(c2)c(-c2ccc4ccccc4c2)c2ccc4nc5c6ccc(-c7ccccc7)cc6c(-c6ccc7ccccc7c6)c6ccc7nc3c2c4c7c65)cc1 ; CC(C)(C)c1ccc(-c2ccc3c(c2)c(-c2ccc4ccccc4c2)c2ccc4nc5c6ccc(-c7ccc(C(C)(C)C)cc7)cc6c(-c6ccc7ccccc7c6)c6ccc7nc3c2c4c7c65)cc1]

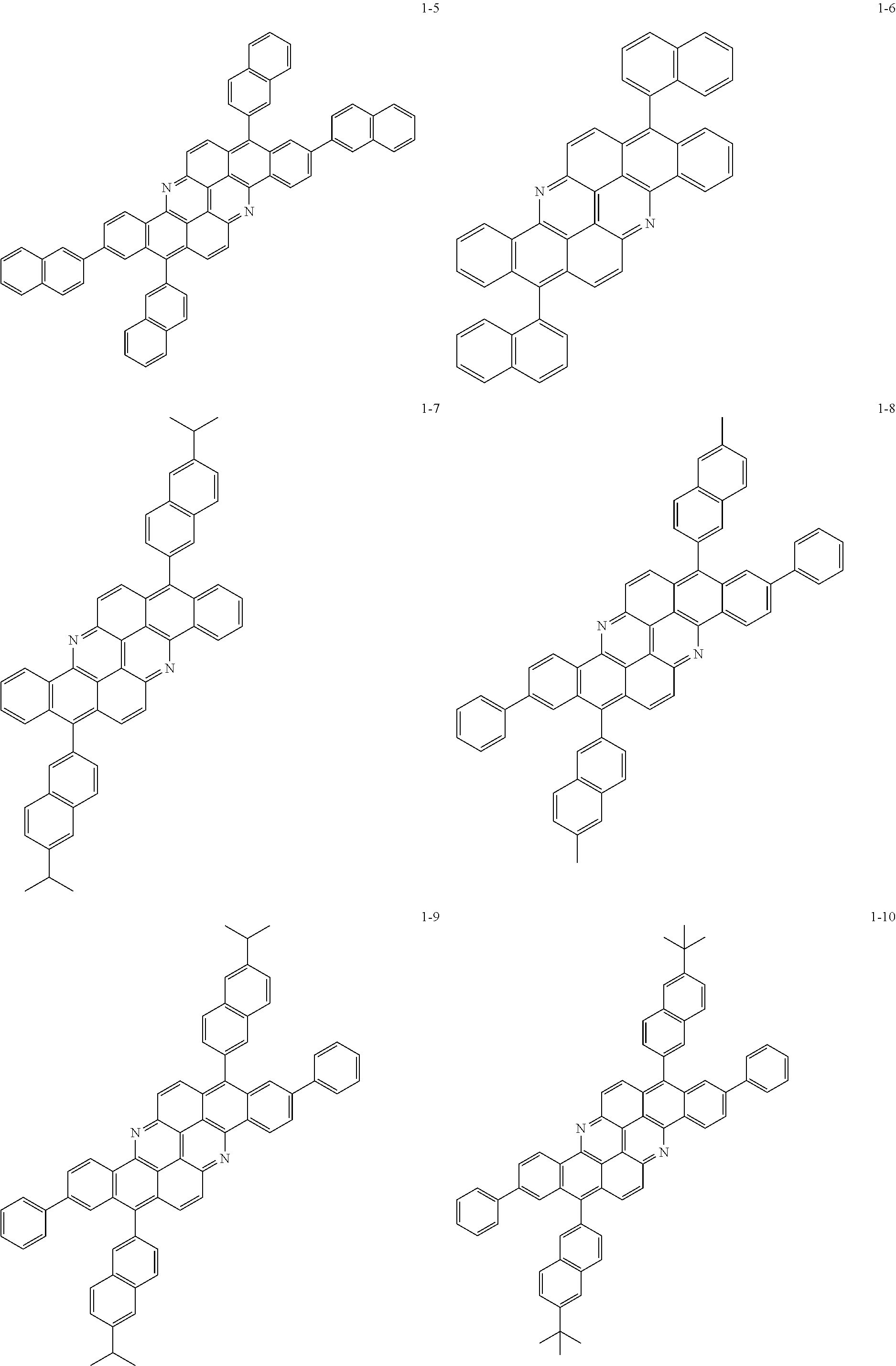

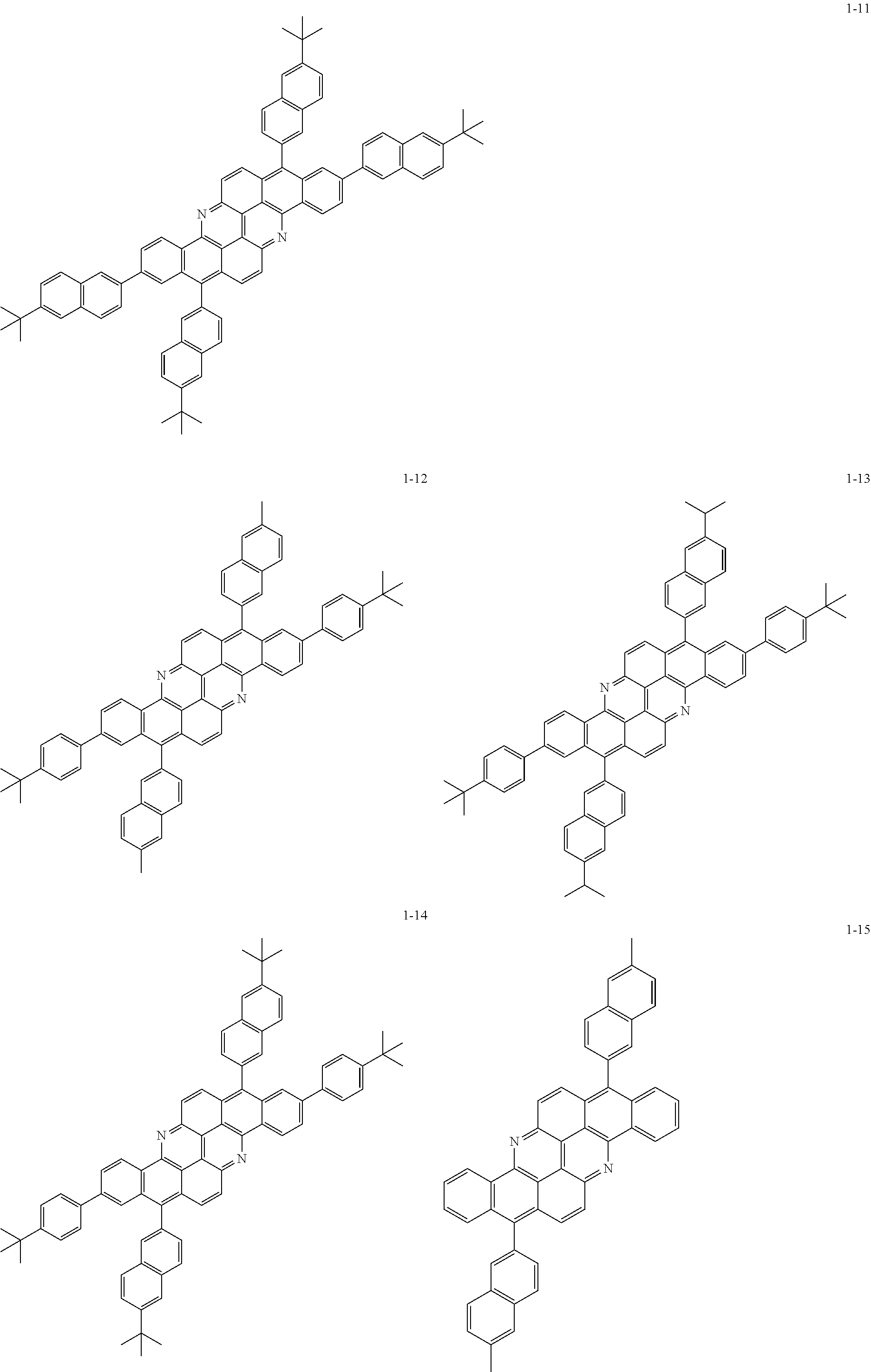

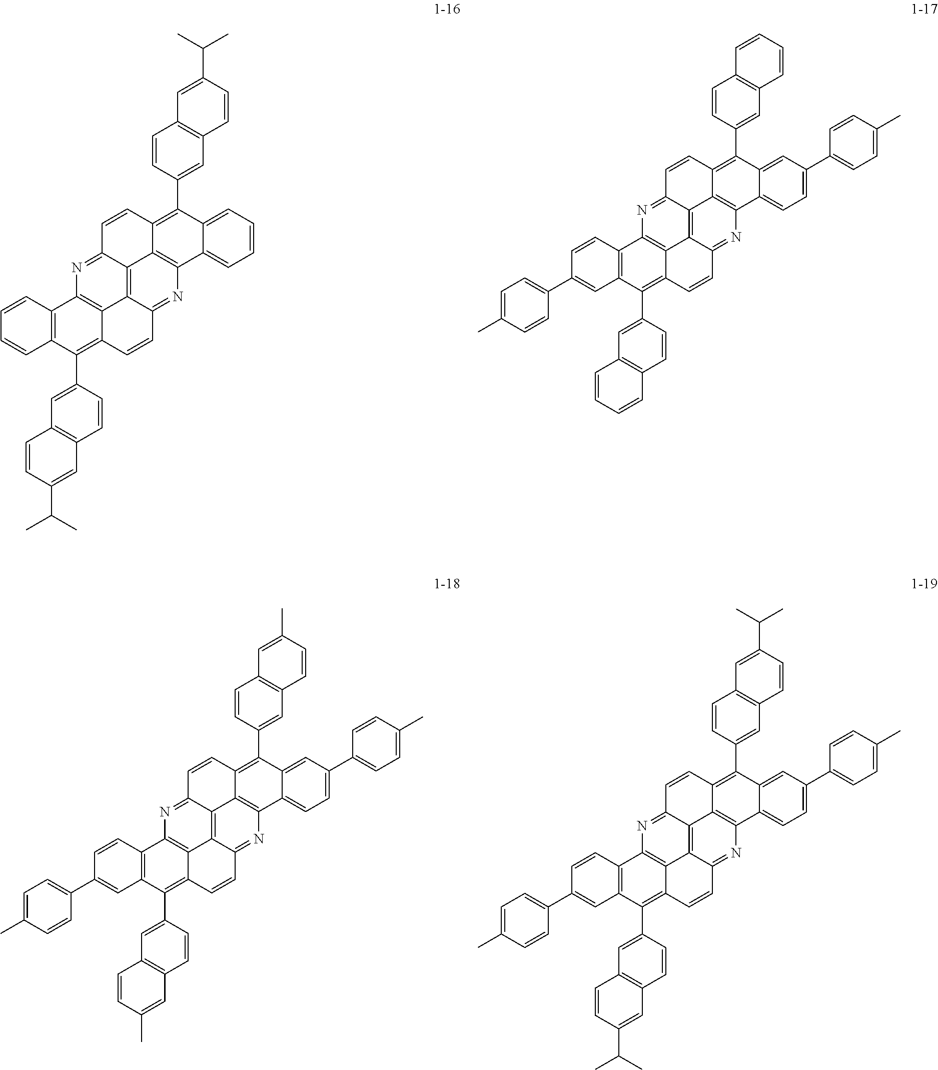

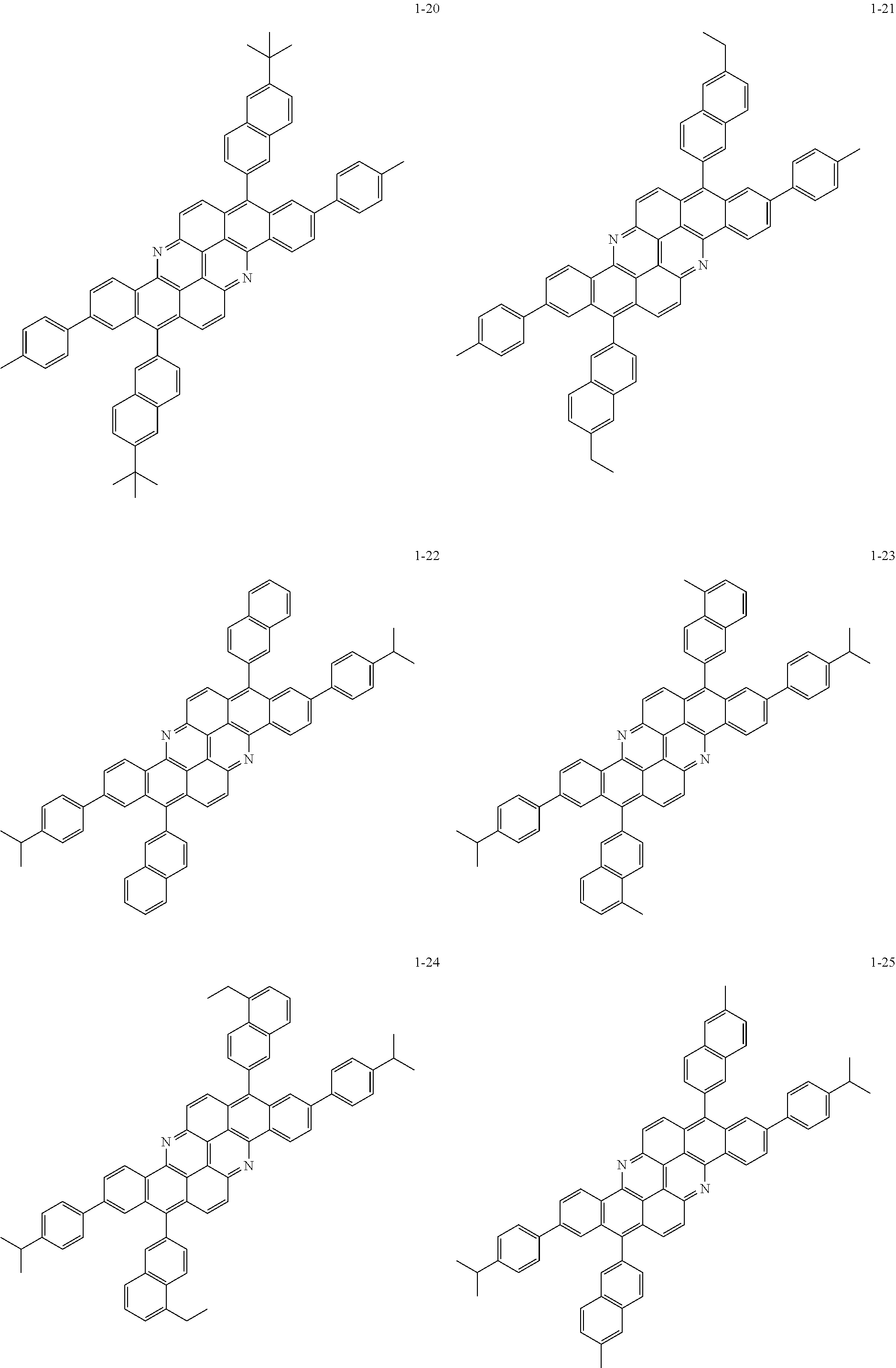

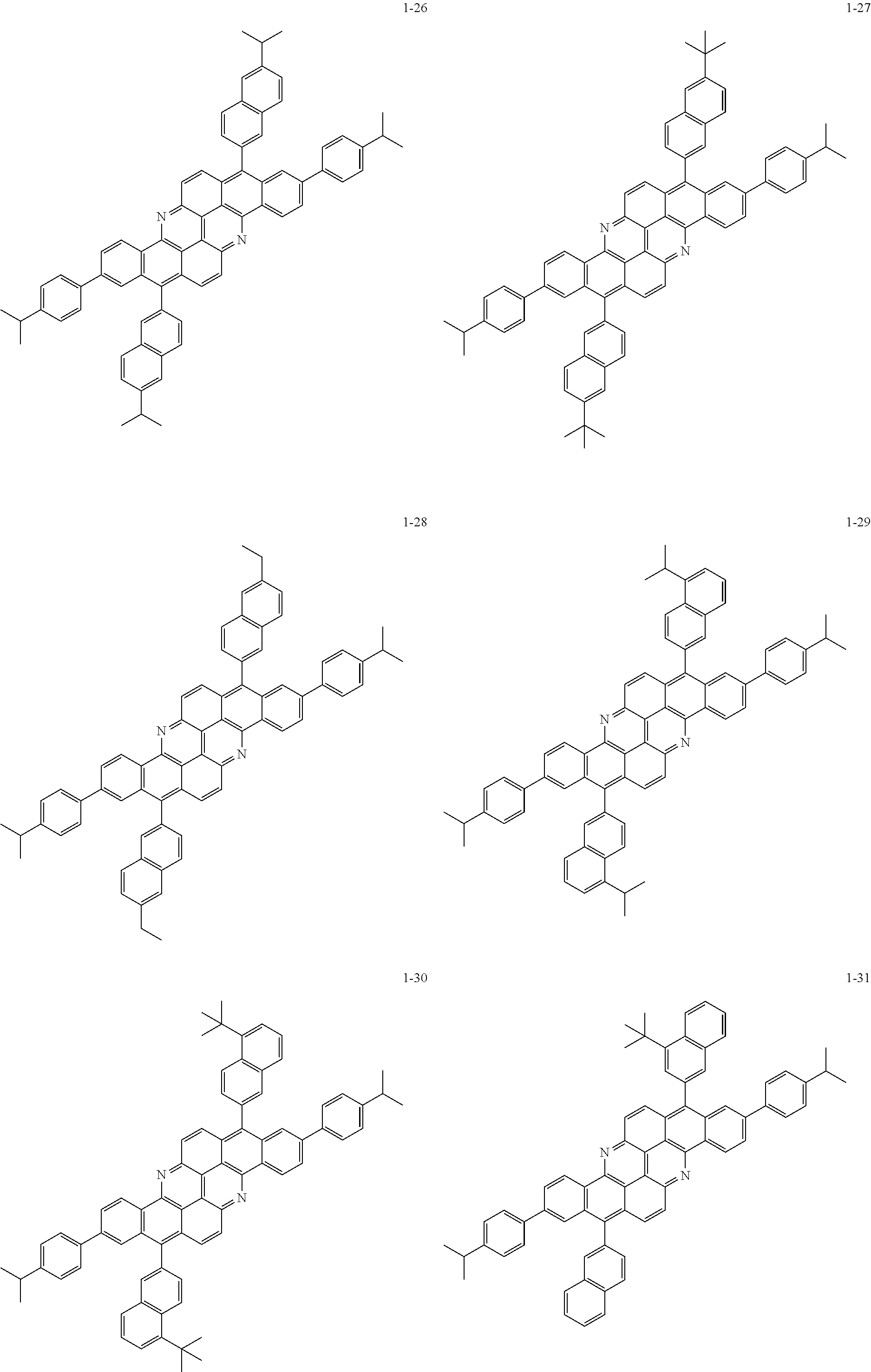

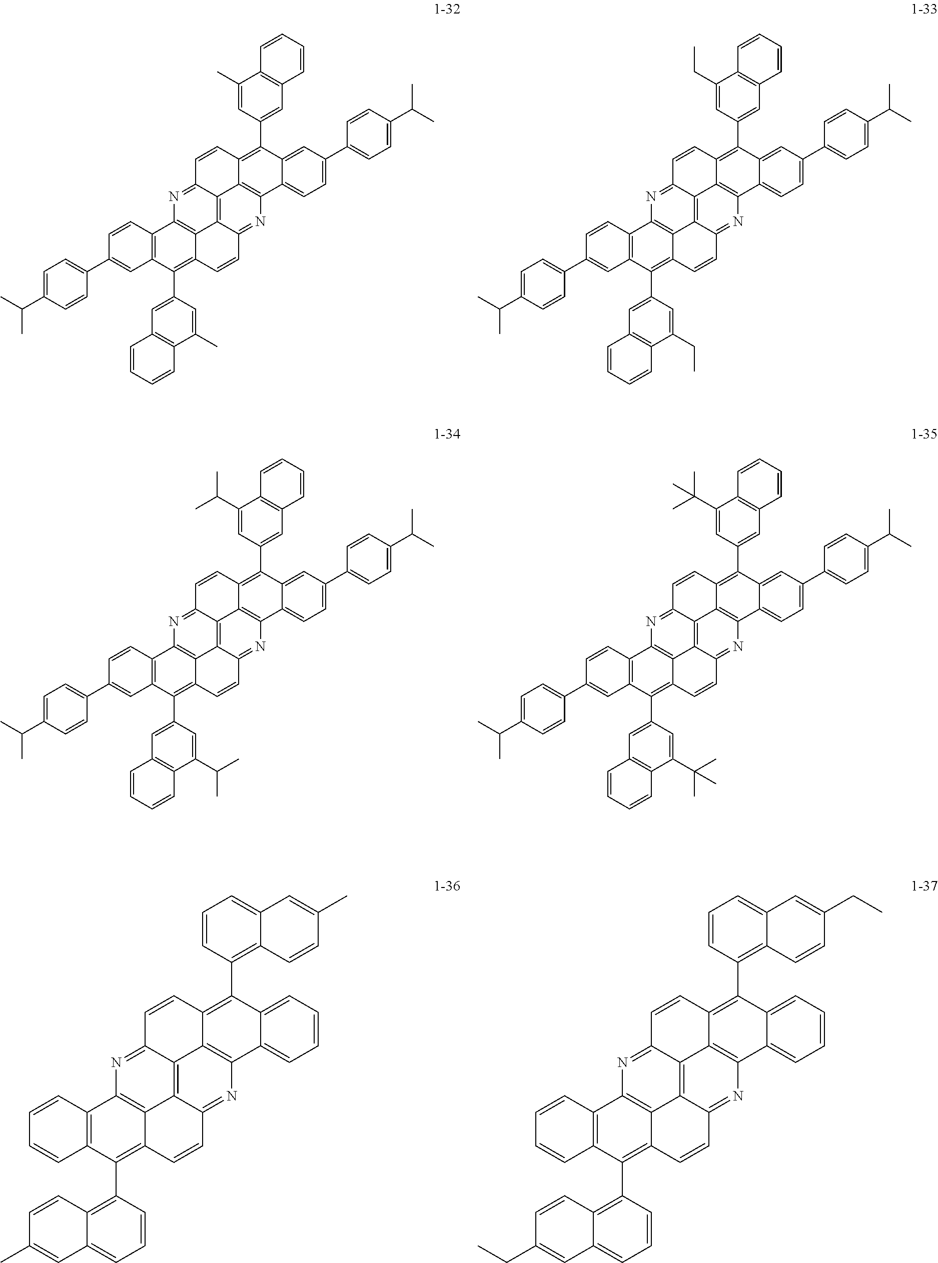

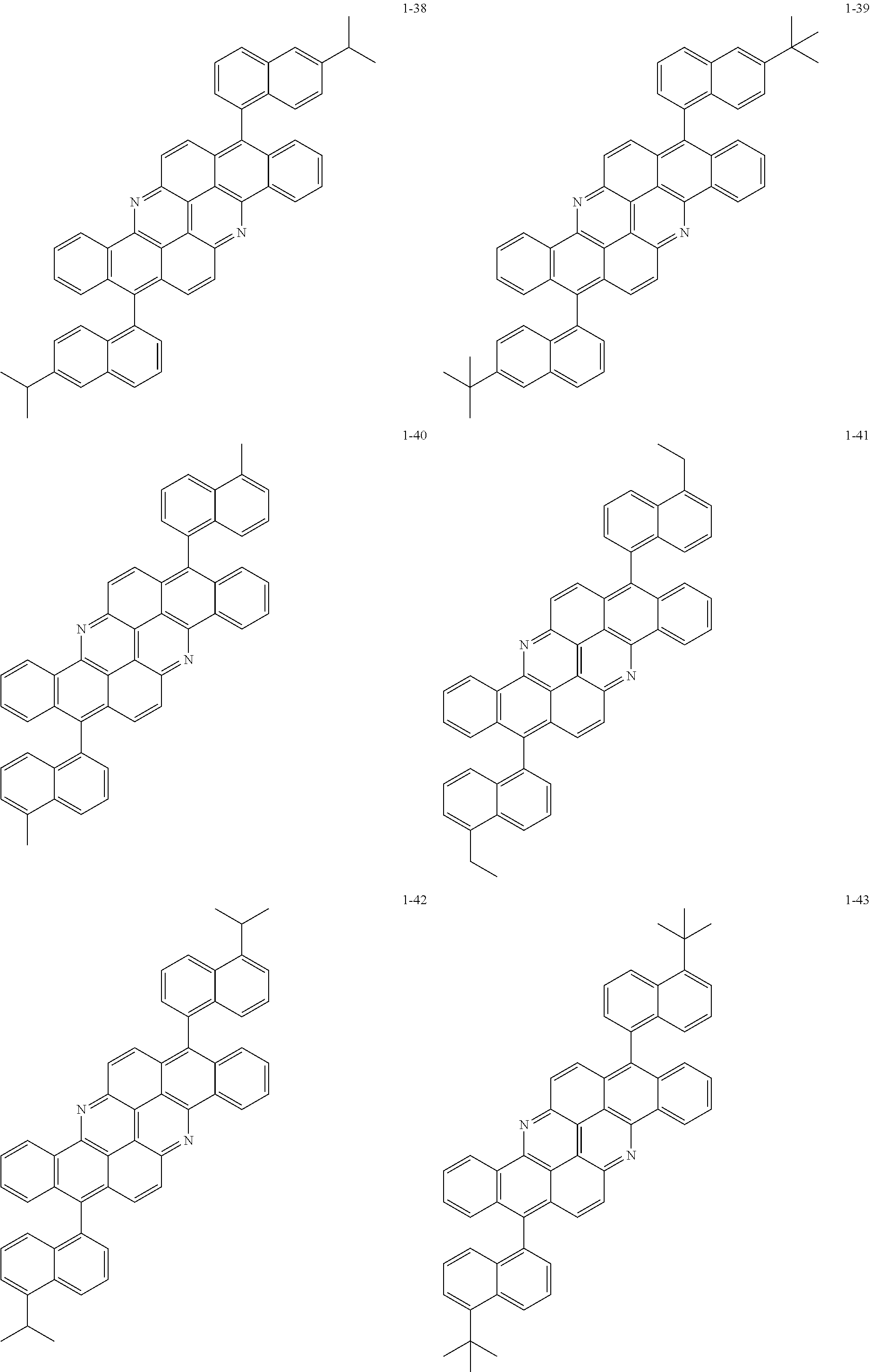

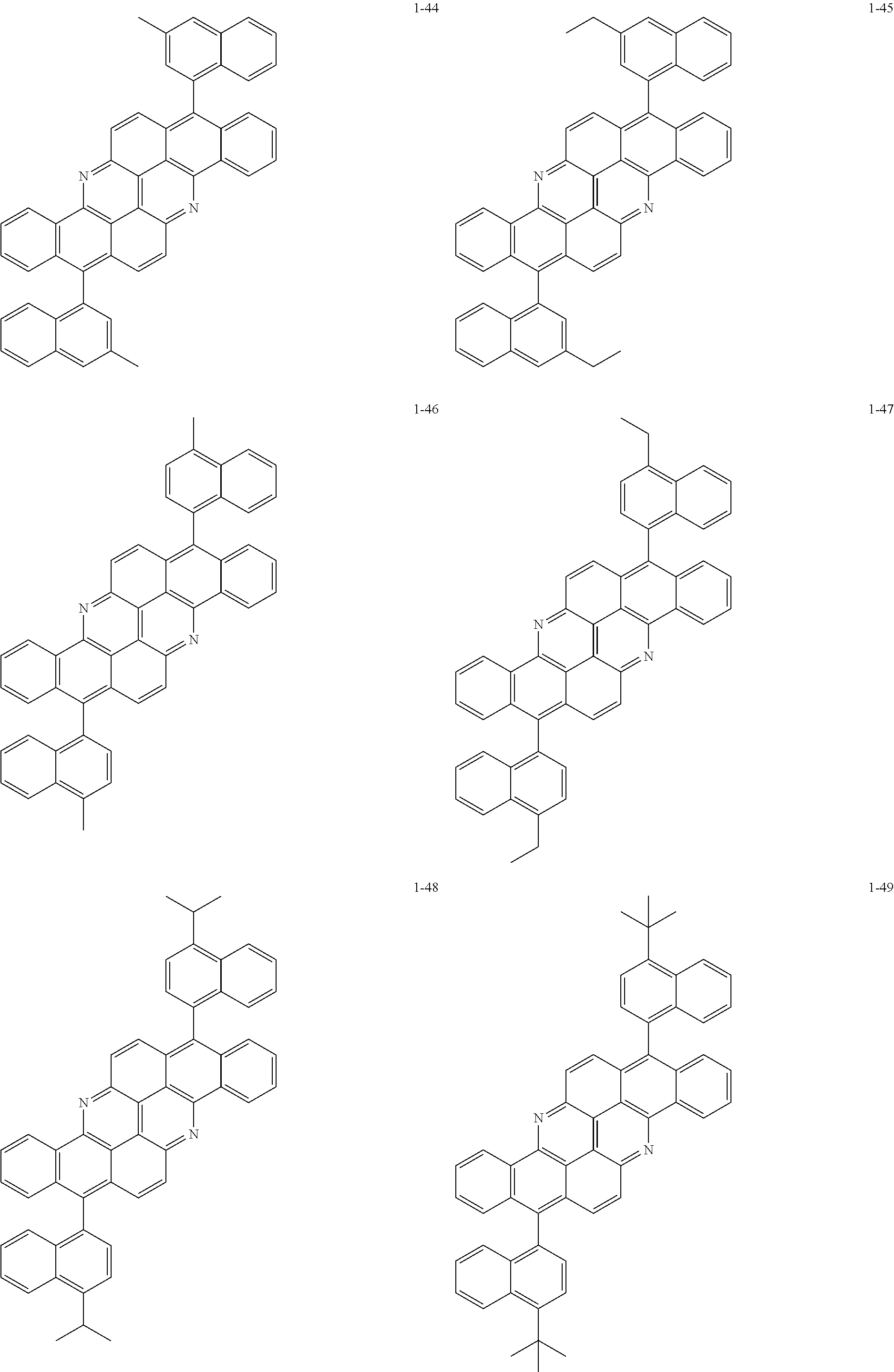

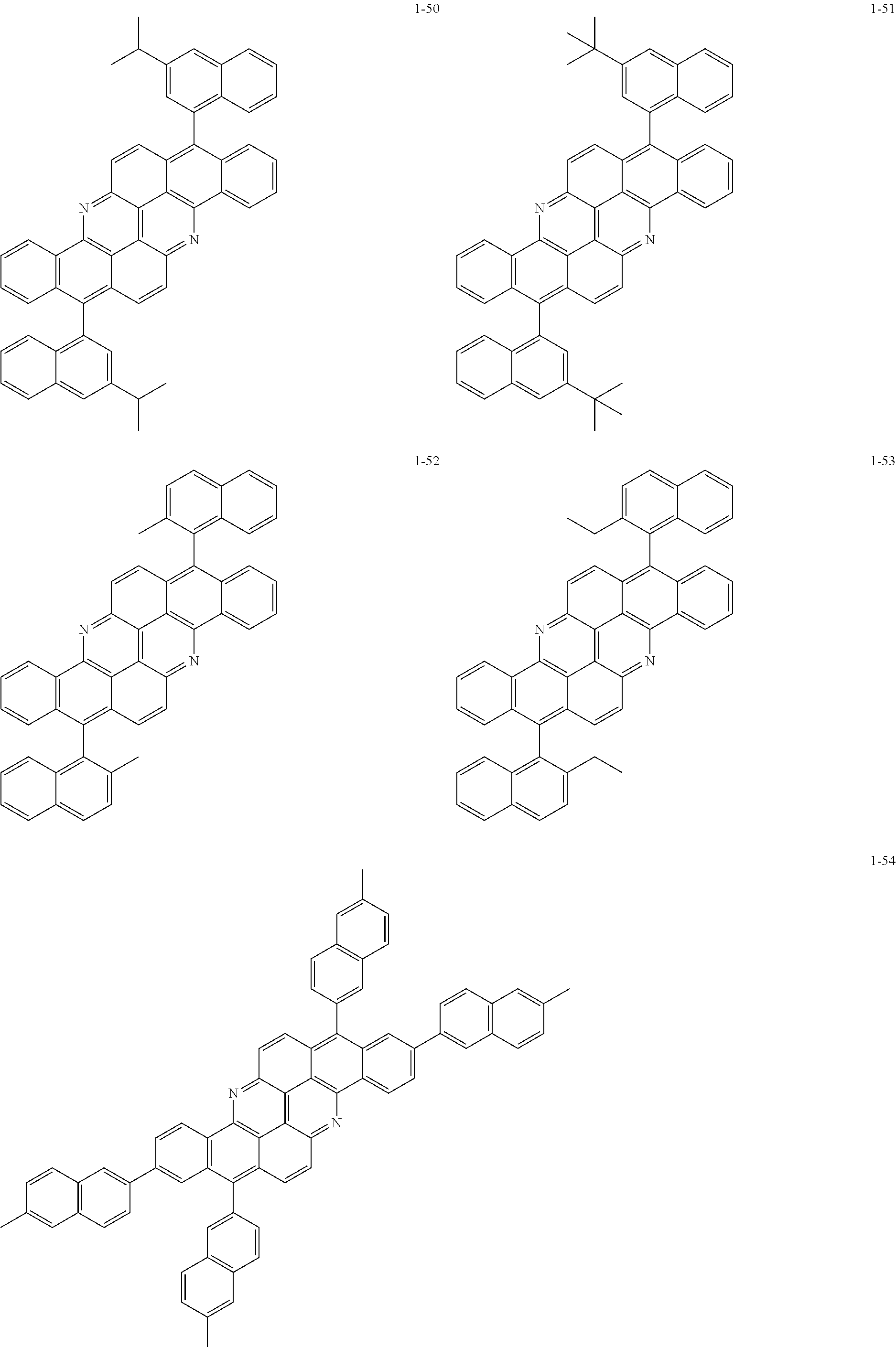

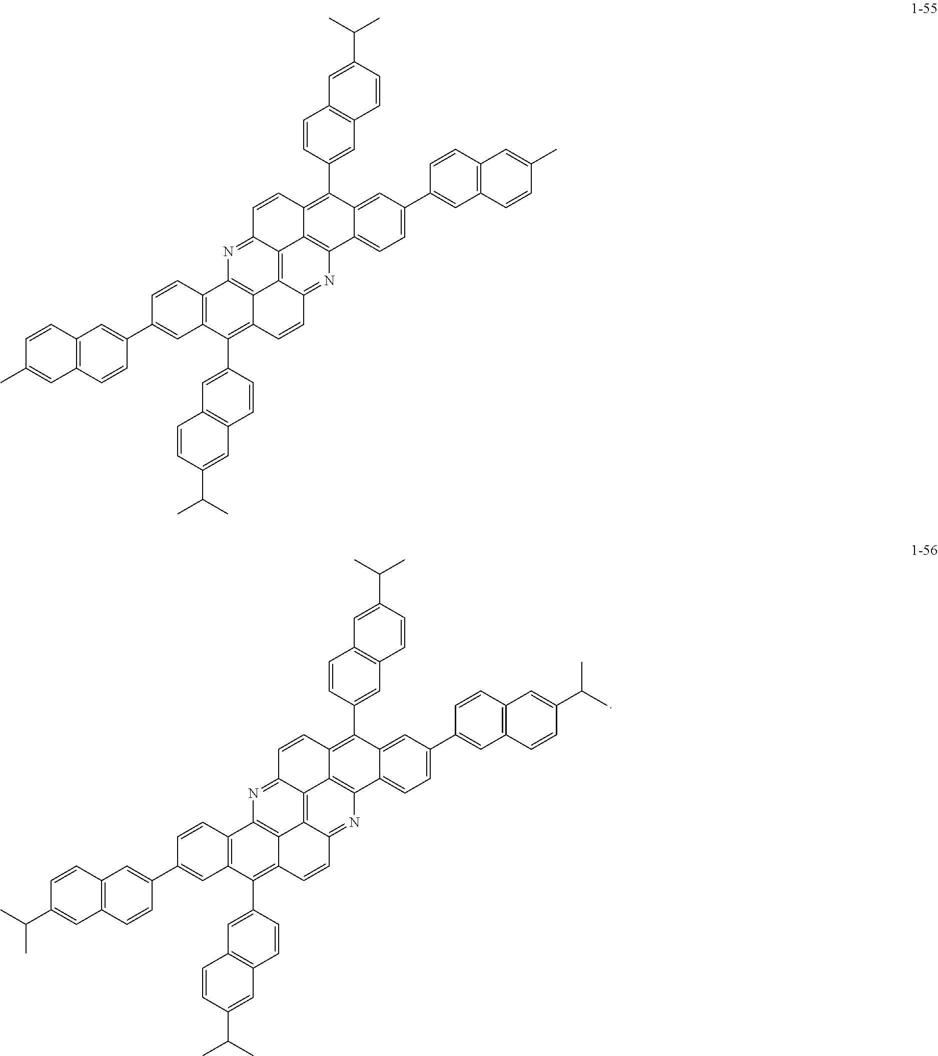

The organic compound having the structure of Chemical Formulae 1 to 3 comprises fused ring system of multiple aromatic or hetero aromatic rings to have a wide plate-like structure. The singlet exciton energy of the host and/or other luminous materials can be transferred efficiently to the singlet exciton of the organic compound having the structure of Chemical Formulae 1 to 3. The organic light emitting diode can realize beneficial luminous efficiency and luminous lifespan by introducing the organic compound having the structure of Chemical Formulae 1 to 3 into the emissive layer.

### [Organic Light Emitting Diode and Organic Light Emitting Device]

In some embodiments, an emissive layer including the organic compound can be applied to an organic light emitting diode having a single emitting part in a red pixel region. Alternatively, the emissive layer including the organic compound can be applied to an organic light emitting diode having a tandem structure where at least two emitting parts are stacked.

The organic light emitting diode where an emissive layer comprises the organic compound can be applied to an organic light emitting device such as an organic light emitting display device or an organic light emitting illumination device. As an example, an organic light emitting display device will be described.

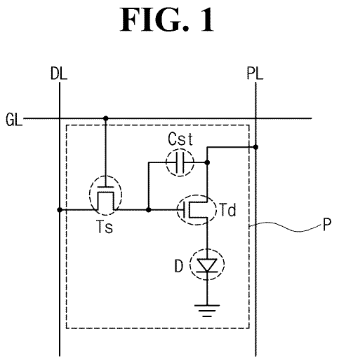

FIG. 1 illustrates a schematic circuit diagram of an organic light emitting display device in accordance with the present disclosure. As illustrated in FIG. 1, a gate line GL, a data line DL and power line PL, each of which crosses each other to define a pixel region P, in an organic light emitting display device. A switching thin film transistor Ts, a driving thin film transistor Td, a storage capacitor Cst and an organic light emitting diode D are disposed within the pixel region P. The pixel region P may comprise a red (R) pixel region, a green (G) pixel region and a blue (B) pixel region. However, embodiments of the present disclosure are not limited to such examples.

The switching thin film transistor Ts is connected to the gate line GL and the data line DL. The driving thin film transistor Td and the storage capacitor Cst are connected between the switching thin film transistor Ts and the power line PL. The organic light emitting diode D is connected to the driving thin film transistor Td. When the switching thin film transistor Ts is turned on by a gate signal applied to the gate line GL, a data signal applied to the data line DL is applied to a gate electrode of the driving thin film transistor Td and one electrode of the storage capacitor Cst through the switching thin film transistor Ts.

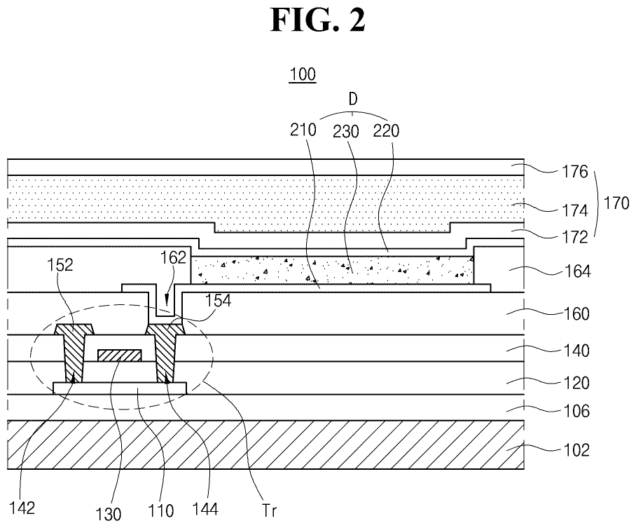

The driving thin film transistor Td is turned on by the data signal applied to the gate electrode 130 (FIG. 2) so that a current proportional to the data signal is supplied from the power line PL to the organic light emitting diode D through the driving thin film transistor Td. And then, the organic light emitting diode D emits light having a luminance proportional to the current flowing through the driving thin film transistor Td. In this case, the storage capacitor Cst is charged with a voltage proportional to the data signal so that the voltage of the gate electrode in the driving thin film transistor Td is kept constant during one frame. Therefore, the organic light emitting display device can display a desired image.

FIG. 2 illustrates a schematic cross-sectional view of an organic light emitting display device in accordance with an example embodiment of the present disclosure. As illustrated in FIG. 2, the organic light emitting display device 100 comprises a substrate 102, a thin-film transistor Tr on the substrate 102, and an organic light emitting diode D connected to the thin film transistor Tr.

As an example, the substrate 102 can comprise a red pixel region, a green pixel region and a blue pixel region and an organic light emitting diode D can be located in each pixel region P. Each of the organic light emitting diodes D emitting red, green and blue light, respectively, is located correspondingly in the red pixel region, the green pixel region and the blue pixel region.

The substrate 102 can comprise, but is not limited to, glass, thin flexible material and/or polymer plastics. For example, the flexible material can include at least one selected from the group consisting of, but is not limited to, polyimide (PI), polyethersulfone (PES), polyethylenenaphthalate (PEN), polyethylene terephthalate (PET), polycarbonate (PC) and combinations thereof. The substrate 102, on which the thin film transistor Tr and the organic light emitting diode D are arranged, forms an array substrate.

A buffer layer 106 can be disposed on the substrate 102. The thin film transistor Tr can be disposed on the buffer layer 106. The buffer layer 106 can be omitted.

A semiconductor layer 110 is disposed on the buffer layer 106. In some example embodiments, the semiconductor layer 110 can comprise, but is not limited to, oxide semiconductor materials. In this case, a light-shield pattern may be disposed under the semiconductor layer 110, and the light-shield pattern can prevent light from being incident toward the semiconductor layer 110, and thereby, preventing or reducing the semiconductor layer 110 from being degraded by the light. Alternatively, the semiconductor layer 110 can comprise polycrystalline silicon. In this case, opposite edges of the semiconductor layer 110 can be doped with impurities.

A gate insulating layer 120 including an insulating material is disposed on the semiconductor layer 110. The gate insulating layer 120 can comprise, but is not limited to, an inorganic insulating material such as silicon oxide (SiOₓ, wherein 0<x≤2) or silicon nitride (SiNₓ, wherein 0<x≤2).

A gate electrode 130 made of a conductive material such as a metal is disposed on the gate insulating layer 120 so as to correspond to a center of the semiconductor layer 110. While the gate insulating layer 120 is disposed on an entire area of the substrate 102 as shown in FIG. 2, the gate insulating layer 120 may be patterned identically as the gate electrode 130.

An interlayer insulating layer 140 including an insulating material is disposed on the gate electrode 130 and covers an entire surface of the substrate 102. The interlayer insulating layer 140 can comprise, but is not limited to, an inorganic insulating material such as silicon oxide (SiOₓ) or silicon nitride (SiNₓ), or an organic insulating material such as benzocyclobutene or photo-acryl.

The interlayer insulating layer 140 has first and second semiconductor layer contact holes 142 and 144 that expose or do not cover a portion of the surface nearer to the opposing ends than to a center of the semiconductor layer 110. The first and second semiconductor layer contact holes 142 and 144 are disposed on opposite sides of the gate electrode 130 and spaced apart from the gate electrode 130. The first and second semiconductor layer contact holes 142 and 144 are formed within the gate insulating layer 120 as illustrated in FIG. 2. Alternatively, the first and second semiconductor layer contact holes 142 and 144 can be formed only within the interlayer insulating layer 140 when the gate insulating layer 120 is patterned identically as the gate electrode 130.

A source electrode 152 and a drain electrode 154, which are made of conductive material such as a metal, are disposed on the interlayer insulating layer 140. The source electrode 152 and the drain electrode 154 are spaced apart from each other on opposing sides of the gate electrode 130, and contact both sides of the semiconductor layer 110 through the first and second semiconductor layer contact holes 142 and 144, respectively.

The semiconductor layer 110, the gate electrode 130, the source electrode 152 and the drain electrode 154 constitute the thin film transistor Tr, which acts as a driving element. The thin film transistor Tr in FIG. 2 has a coplanar structure in which the gate electrode 130, the source electrode 152 and the drain electrode 154 are disposed on the semiconductor layer 110. Alternatively, the thin film transistor Tr can have an inverted staggered structure in which a gate electrode is disposed under a semiconductor layer and a source and drain electrodes are disposed on the semiconductor layer. In this case, the semiconductor layer can comprise amorphous silicon.

The gate line GL and the data line DL, which cross each other to define a pixel region P, and a switching element Ts, which is connected to the gate line GL and the data line DL, can be further formed in the pixel region P. The switching element Ts is connected to the thin film transistor Tr, which is a driving element. In addition, the power line PL is spaced apart in parallel from the gate line GL or the data line DL. The thin film transistor Tr may further comprise a storage capacitor Cst configured to constantly keep a voltage of the gate electrode 130 for one frame.

A passivation layer 160 is disposed on the source and drain electrodes 152 and 154. The passivation layer 160 covers the thin film transistor Tr on the entire substrate 102. The passivation layer 160 has a flat top surface and a drain contact hole 162 that exposes or does not cover the drain electrode 154 of the thin film transistor Tr. While the drain contact hole 162 is disposed on the second semiconductor layer contact hole 144, it may be spaced apart from the second semiconductor layer contact hole 144.

The organic light emitting diode (OLED) D comprises a first electrode 210 that is disposed on the passivation layer 160 and connected to the drain electrode 154 of the thin film transistor Tr. The OLED D further comprises an emissive layer 230 and a second electrode 220 each of which is disposed sequentially on the first electrode 210.

The first electrode 210 is disposed in each pixel region. The first electrode 210 can be an anode and comprise conductive material having relatively high work function value. For example, the first electrode 210 can comprise a transparent conductive oxide (TCO). More particularly, the first electrode 210 can comprise, but is not limited to, indium tin oxide (ITO), indium zinc oxide (IZO), indium tin zinc oxide (ITZO), tin oxide (SnO), zinc oxide (ZnO), indium cerium oxide (ICO), aluminum doped zinc oxide (AZO), and/or combinations thereof.

In one example embodiment, when the organic light emitting display device 100 is a bottom-emission type, the first electrode 210 can have a single-layered structure of the TCO. Alternatively, when the organic light emitting display device 100 is a top-emission type, a reflective electrode or a reflective layer may be disposed under the first electrode 210. For example, the reflective electrode or the reflective layer can comprise, but is not limited to, silver (Ag) or aluminum-palladium-copper (APC) alloy. In the OLED D of the top-emission type, the first electrode 210 can have a triple-layered structure of ITO/Ag/ITO or ITO/APC/ITO.

In addition, a bank layer 164 is disposed on the passivation layer 160 in order to cover edges of the first electrode 210. The bank layer 164 exposes or does not cover a center of the first electrode 210 corresponding to each pixel region. In certain embodiment, the bank layer 164 can be omitted.

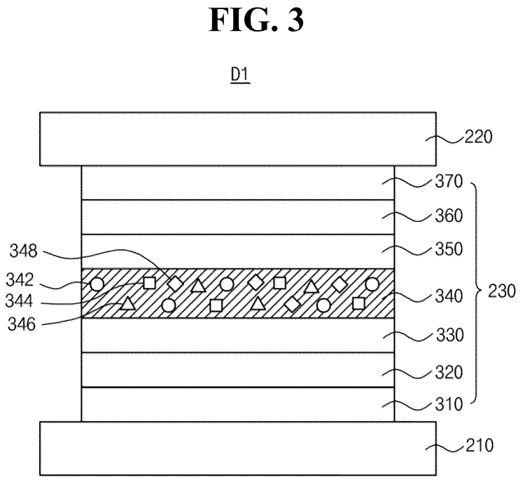

An emissive layer 230 is disposed on the first electrode 210. In some example embodiments, the emissive layer 230 can have a single-layered structure of an emitting material layer (EML). Alternatively, the emissive layer 230 can have a multiple-layered structure of a hole injection layer (HIL), a hole transport layer (HTL), an electron blocking layer (EBL), an EML, a hole blocking layer (HBL), an electron transport layer (ETL), an electron injection layer (EIL) and/or a charge generation layer (CGL) as illustrated in FIG. 3. In one aspect, the emissive layer 230 can have a single emitting part. Alternatively, the emissive layer 230 can have multiple emitting parts to form a tandem structure. For example, the emissive layer 230 can be applied to an OLED with a single emitting part located each of the red pixel region, the green pixel region and the blue pixel region. Alternatively, the emissive layer 230 can be applied to a tandem-type OLED where at least two emitting parts are stacked.

The emissive layer 230 can comprise the organic compound having at least one of the structure of Chemical Formulae 1 to 3. The luminous efficiency and the luminous lifespan of the OLED D and the organic light emitting display device 100 can be improved by including the organic compound.

The second electrode 220 is disposed on the substrate 102 above which the emissive layer 230 is disposed. The second electrode 220 can be disposed on an entire display area. The second electrode 220 can comprise a conductive material with a relatively low work function value compared to the first electrode 210. The second electrode 220 can be a cathode providing electrons. For example, the second electrode 220 can comprise at least one selected from the group consisting of, but is not limited to, aluminum (Al), magnesium (Mg), calcium (Ca), silver (Ag), alloy thereof and combinations thereof such as aluminum-magnesium alloy (Al—Mg). When the organic light emitting display device 100 is a top-emission type, the second electrode 220 is thin so as to have light-transmissive (semi-transmissive) property.

In addition, an encapsulation film 170 can be disposed on the second electrode 220 in order to prevent or reduce outer moisture from penetrating into the OLED D. The encapsulation film 170 can have, but is not limited to, a laminated structure of a first inorganic insulating film 172, an organic insulating film 174 and a second inorganic insulating film 176. In certain embodiment, the encapsulation film 170 can be omitted.

A polarizing plate can be attached onto the encapsulation film 170 to reduce reflection of external light. For example, the polarizing plate may be a circular polarizing plate. When the organic light emitting display device 100 is a bottom-emission type, the polarizing plate can be disposed under the substrate 102. Alternatively, when the organic light emitting display device 100 is a top-emission type, the polarizing plate can be disposed on the encapsulation film 170. In addition, a cover window can be attached to the encapsulation film 170 or the polarizing plate. In this case, the substrate 102 and the cover window may have a flexible property, thus the organic light emitting display device 100 may be a flexible display device.

The OLED D is described in more detail. FIG. 3 illustrates a schematic cross-sectional view of an organic light emitting diode having a single emitting part in accordance with some embodiments of the present disclosure. As illustrated in FIG. 3, the organic light emitting diode (OLED) D1 in accordance with the present disclosure comprises first and second electrodes 210 and 220 facing each other and an emissive layer 230 disposed between the first and second electrodes 210 and 220. The organic light emitting display device 100 comprises a red pixel region, a green pixel region and a blue pixel region, and the OLED D1 can be disposed in the red pixel region, the green pixel region and the blue pixel region. As an example, the OLED D1 can be disposed in the red pixel region.

In some embodiments, the emissive layer 230 may comprise an emitting material layer (EML) 340 disposed between the first and second electrodes 210 and 220. Also, the emissive layer 230 can comprise at least one of a hole transport layer (HTL) 320 disposed between the first electrode 210 and the EML 340 or an electron transport layer (ETL) 360 disposed between the second electrode 220 and the EML 340. In addition, the emissive layer 230 can further comprise at least one of a hole injection layer (HIL) 310 disposed between the first electrode 210 and the HTL 320 or an electron injection layer (EIL) 370 disposed between the second electrode 220 and the ETL 360. Alternatively, the emissive layer 230 can further comprise a first exciton blocking layer, i.e. an electron blocking layer (EBL) 330 disposed between the HTL 320 and the EML 340 and/or a second exciton blocking layer, i.e. a hole blocking layer (HBL) 350 disposed between the EML 340 and the ETL 360.

The first electrode 210 can be an anode that provides holes into the EML 340. The first electrode 210 can comprise a conductive material having a relatively high work function value, for example, a transparent conductive oxide (TCO). In an example embodiment, the first electrode 210 can comprise, but is not limited to, ITO, IZO, ITZO, SnO, ZnO, ICO, AZO, and/or combinations thereof.

The second electrode 220 can be a cathode that provides electrons into the EML 340. The second electrode 220 can comprise a conductive material having a relatively low work function values, i.e., a highly reflective material such as Al, Mg, Ca, Ag, and/or alloy thereof and/or combinations thereof such as Al—Mg.

The HIL 310 is disposed between the first electrode 210 and the HTL 320 and can improve an interface property between the inorganic first electrode 210 and the organic HTL 320. In some embodiments, hole injecting material in the HIL 310 can comprise, but is not limited to, 4,4′,4″-tris(3-methylphenylamino)triphenylamine (MTDATA), 4,4′,4″-tris(N,N-diphenyl-amino)triphenylamine (NATA), 4,4′,4″-tris(N-(naphthalene-1-yl)-N-phenyl-amino)triphenylamine (1T-NATA), 4,4′,4″-tris(N-(naphthalene-2-yl)-N-phenyl-amino)triphenylamine (2T-NATA), copper phthalocyanine (CuPc), tris(4-carbazoyl-9-yl-phenyl)amine (TCTA), N,N′-diphenyl-N,N′-bis(1-naphthyl)-1,1′-biphenyl-4,4″-diamine (NPB; NPD), N,N′-bis{4-[bis(3-methylphenyi)amino]phenylI-N,Ndiphenyl-44biphenyidiaine (DNTPD), 1,4,5,8,9,11-hexaazatriphenylenehexacarbonitrile (dipyrazino[2,3-f:2′3′-h]quinoxaline-2,3,6,7,10,11-hexacarbonitrile; HAT-CN), 2,3,5,6-tetrafluoro-7,7,8,8-tetracyanoquinodimethane (F4-TCNQ), 1,3,4,5,7,8-hexafluorotetracyanonaphthoquinodimethane (F6-TCNNQ), 1,3,5-tris[4-(diphenylamino)phenyl]benzene (TDAPB), poly(3,4-ethylenedioxythiphene)polystyrene sulfonate (PEDOT/PSS), N-(biphenyl-4-yl)-9,9-dimethyl-N-(4-(9-phenyl-9H-carbazol-3-yl)phenyl)-9H-fluoren-2-amine, N,N′-diphenyl-N,N′-di[4-(N,N′-diphenyl-amino)phenyl]benzidine (NPNPB) and/or combinations thereof.

In another embodiments, the HIL 310 comprises the hole transporting material below doped with hole injecting material (e.g., HAT-CN, F4-TCNQ and/or F6-TCNNQ). In this case, the contents of the hole injection material in the HIL 310 can be between about 2 wt % and about 15 wt %. In certain embodiment, the HIL 310 can be omitted in compliance of the OLED D1 property.

The HTL 320 is disposed adjacently to the EML 340 between the first electrode 210 and the EML 340. In some embodiments, a hole transporting material in the HTL 320 can comprise, but is not limited to, N,N′-diphenyl-N,N′-bis(3-methylphenyl)-1,1′-biphenyl-4,4′-diamine (TPD), NPB(NPD), DNTPD, 4,4′-bis(N-carbazolyl)-1,1′-biphenyl (CBP), poly[N,N′-bis(4-butylphenyl)-N,N′-bis(phenyl)-benzidine] (Poly-TPD), poly[(9,9-dioctylfluorenyl-2,7-diyl)-co-(4,4′-(N-(4-sec-butylphenyl)diphenylamine))] (TFB), di-[4-(N,N-di-p-tolyl-amino)-phenyl]cyclohexane (TAPC), 3,5-di(9H-carbazol-9-yl)-N,N-diphenylaniline (DCDPA), N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine, N-([1,1′-biphenyl]-4-yl)-9,9-dimethyl-N-(4-(9-phenyl-9H-carbazol-3-yl)phenyl)-9H-fluoren-2-amine and/or combinations thereof.

The EML 340 can comprise an emitter (a dopant) 342 and a first host 344, and optionally, a second host 346, and ultimate emission is occurred at the emitter 342. The EML 340 can emit red to yellow-green color light, for example, red color light. The emitter 342 can comprise at least one of the organic compounds having the structure of Chemical Formulae 1 to 3.

The first host 344 can be a P-type host with relatively strong hole affinity properties. For example, the first host 344 can comprise, but is not limited to, a carbazole-based organic compound, an aryl and/or hetero aryl amino-based organic compound with at least one fused aromatic and/or fused hetero aromatic moiety and/or an aryl and/or hetero aryl amino-based organic compound with at least one spirofluorene moiety.

The second host 346 can be an N-type host with relatively strong electron affinity properties. For example, the second host 346 can comprise, but is not limited to, an azine-based organic compound and a quinazoline-based organic compound.

In some embodiments, the first host 344 can be the carbazole-based organic compound. For example, the first host 344 can comprise a compound having the following structure of Chemical Formula 4:

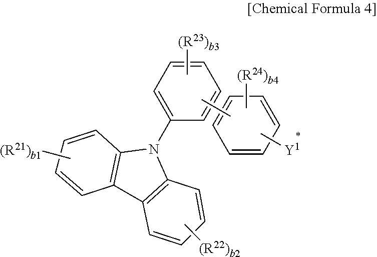

wherein, in Chemical Formula 4,
each of R²¹ and R²² is independently an unsubstituted or substituted C₁-C₂₀ alkyl group or an unsubstituted or substituted C₆-C₃₀ aryl group, where each R²¹ is identical to or different from each other when b1 is 2, 3 or 4, and each R²² is identical to or different from each other when b2 is 2, 3 or 4;
each of R²³ and R²⁴ is independently an unsubstituted or substituted C₁-C₂₀ alkyl group or an unsubstituted or substituted C₆-C₃₀ aryl group, where each R²³ is identical to or different from each other when b3 is 2, 3 or 4, and each R²⁴ is identical to or different from each other when b4 is 2, 3 or 4, or optionally,
optionally, R²³ and R²⁴ may be linked together to form an unsubstituted or substituted hetero ring;
Y¹ has the following structure of Chemical Formula 5A or Chemical Formula 5B;
each of b1, b2, b3 and b4 is independently 0, 1, 2, 3 or 4; and
the asterisk indicates a link to the following Chemical Formula 5A or Chemical Formula 5B:

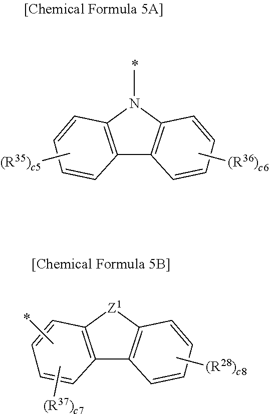

wherein, in Chemical Formulae 5A and 5B,
each of R³⁵, R³⁶, R³⁷ and R³⁸ is independently an unsubstituted or substituted C₁-C₂₀ alkyl group or an unsubstituted or substituted C₆-C₃₀ aryl group, where each R³⁵ is identical to or different from each other when c5 is 2, 3 or 4, each R³⁶ is identical to or different from each other when c6 is 2, 3 or 4, each R³⁷ is identical to or different from each other when c7 is 2 or 3, and each R³⁸ is identical to or different from each other when c8 is 2, 3 or 4;
Z¹ is NR³⁹, O or S, where R³⁹ is hydrogen, an unsubstituted or substituted C₁-C₂₀ alkyl group or an unsubstituted or substituted C₆-C₃₀ aryl group;
each of c5, c6 and c8 is independently 0, 1, 2, 3 or 4;
c7 is 0, 1, 2 or 3; and
asterisk indicates a link position.

For example, the carbazolyl moiety including R²¹ and R²² and the phenyl moiety including R²⁴ in Chemical Formula 4 can be linked to an ortho-, meta- or para- position to the benzene ring with R²³. In addition, R²³ and R²⁴ are further linked together to form a 5-membered hetero aromatic ring including a nitrogen atom, an oxygen atom and/or a sulfur atom. The nitrogen atom in the 5-membered hetero aromatic ring formed by R²³ and R²⁴ can be unsubstituted or substituted with a C₆-C₂₀ aryl group (e.g., phenyl).

As an example, the first host 344 having the structure of Chemical Formula 4 can be, but is not limited to, at least one of the following organic compounds of Chemical Formula 6 (compounds 2-1 to 2-24):

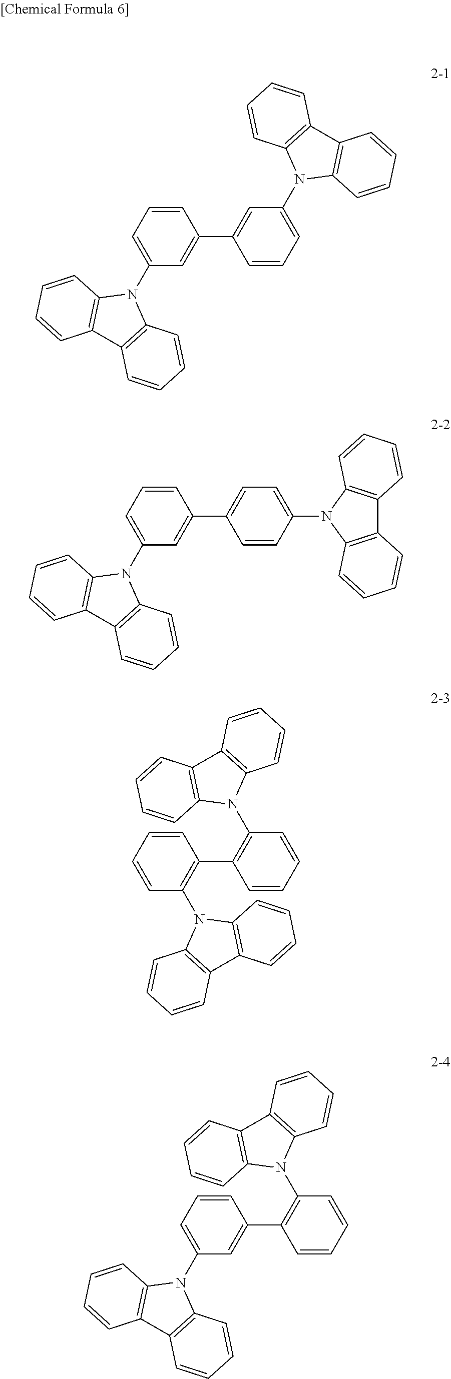 [SMILES: c1cc(-c2cccc(-n3c4ccccc4c4ccccc43)c2)cc(-n2c3ccccc3c3ccccc32)c1 ; c1cc(-c2ccc(-n3c4ccccc4c4ccccc43)cc2)cc(-n2c3ccccc3c3ccccc32)c1 ; c1ccc(-n2c3ccccc3c3ccccc32)c(-c2ccccc2-n2c3ccccc3c3ccccc32)c1 ; c1cc(-c2ccccc2-n2c3ccccc3c3ccccc32)cc(-n2c3ccccc3c3ccccc32)c1]

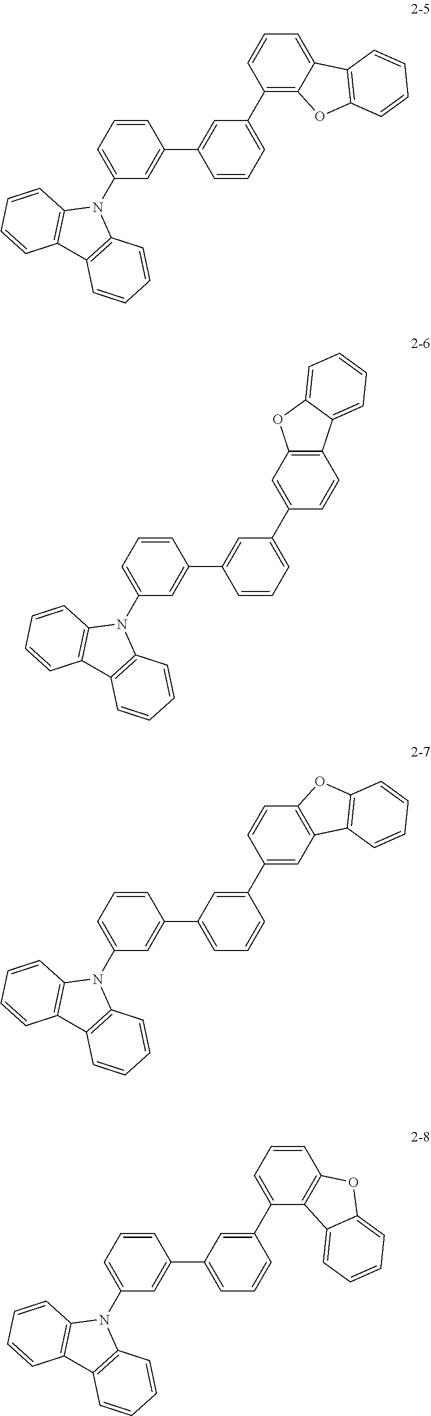 [SMILES: c1cc(-c2cccc(-n3c4ccccc4c4ccccc43)c2)cc(-c2cccc3c2oc2ccccc23)c1 ; c1cc(-c2cccc(-n3c4ccccc4c4ccccc43)c2)cc(-c2ccc3c(c2)oc2ccccc23)c1 ; c1cc(-c2cccc(-n3c4ccccc4c4ccccc43)c2)cc(-c2ccc3oc4ccccc4c3c2)c1 ; c1cc(-c2cccc(-n3c4ccccc4c4ccccc43)c2)cc(-c2cccc3oc4ccccc4c23)c1]

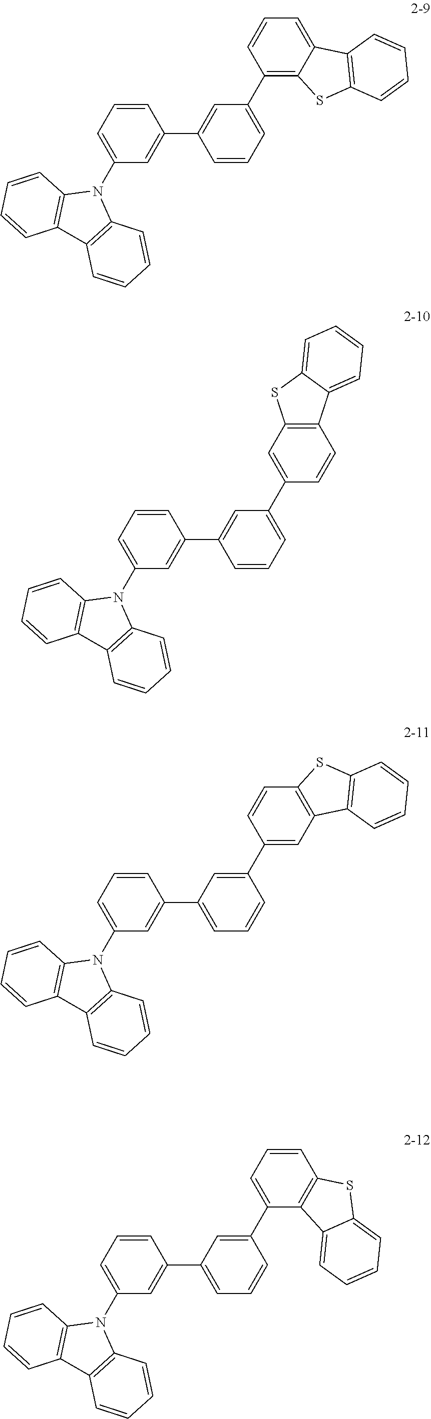 [SMILES: c1cc(-c2cccc(-n3c4ccccc4c4ccccc43)c2)cc(-c2cccc3c2sc2ccccc23)c1 ; c1cc(-c2cccc(-n3c4ccccc4c4ccccc43)c2)cc(-c2ccc3c(c2)sc2ccccc23)c1 ; c1cc(-c2cccc(-n3c4ccccc4c4ccccc43)c2)cc(-c2ccc3sc4ccccc4c3c2)c1 ; c1cc(-c2cccc(-n3c4ccccc4c4ccccc43)c2)cc(-c2cccc3sc4ccccc4c23)c1]

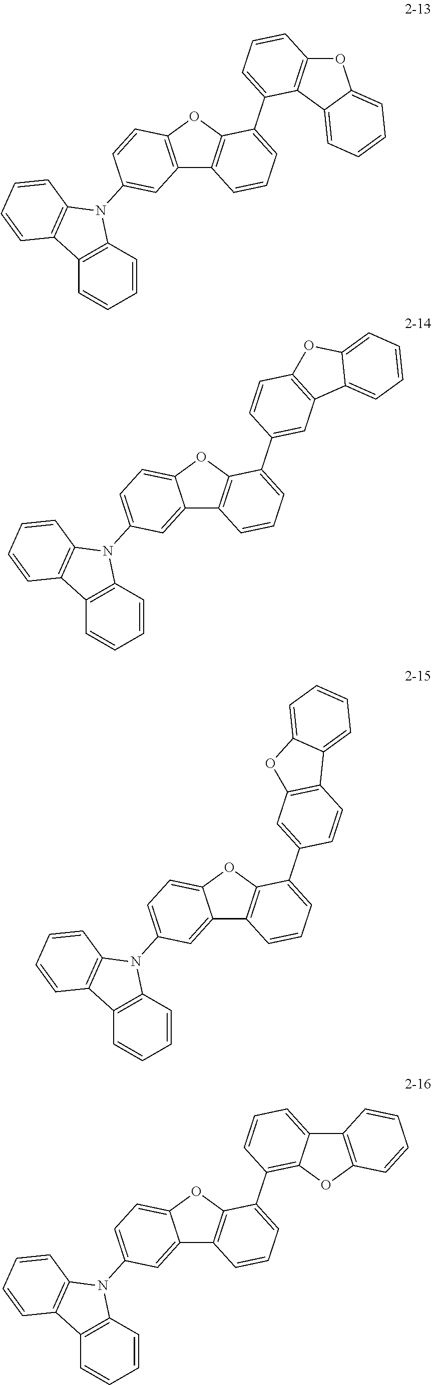 [SMILES: c1ccc2c(c1)oc1cccc(-c3cccc4c3oc3ccc(-n5c6ccccc6c6ccccc65)cc34)c12 ; c1ccc2c(c1)oc1ccc(-c3cccc4c3oc3ccc(-n5c6ccccc6c6ccccc65)cc34)cc12 ; c1ccc2c(c1)oc1cc(-c3cccc4c3oc3ccc(-n5c6ccccc6c6ccccc65)cc34)ccc12 ; c1ccc2c(c1)oc1c(-c3cccc4c3oc3ccc(-n5c6ccccc6c6ccccc65)cc34)cccc12]

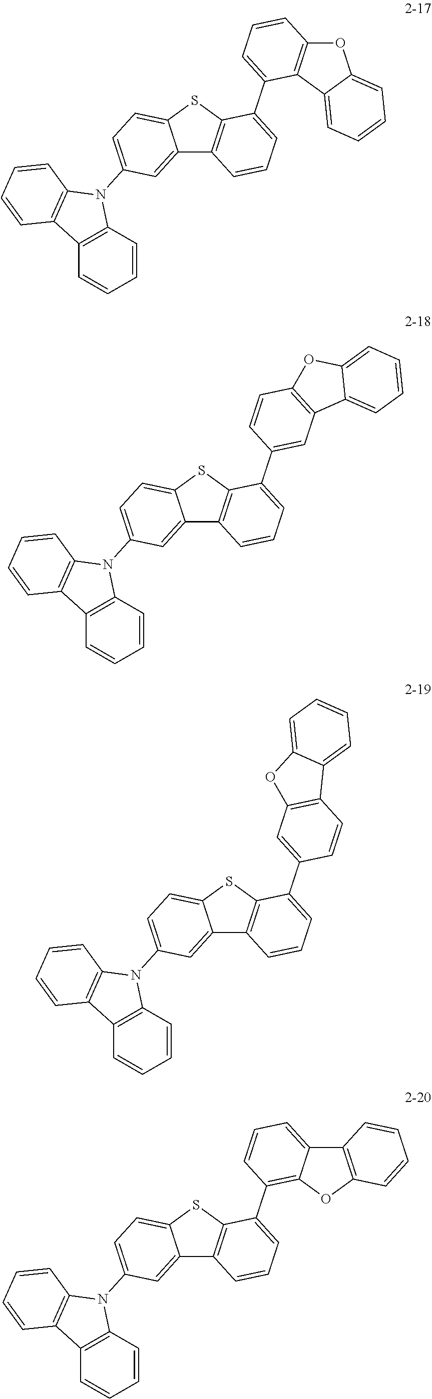 [SMILES: c1ccc2c(c1)oc1cccc(-c3cccc4c3sc3ccc(-n5c6ccccc6c6ccccc65)cc34)c12 ; c1ccc2c(c1)oc1ccc(-c3cccc4c3sc3ccc(-n5c6ccccc6c6ccccc65)cc34)cc12 ; c1ccc2c(c1)oc1cc(-c3cccc4c3sc3ccc(-n5c6ccccc6c6ccccc65)cc34)ccc12 ; c1ccc2c(c1)oc1c(-c3cccc4c3sc3ccc(-n5c6ccccc6c6ccccc65)cc34)cccc12]

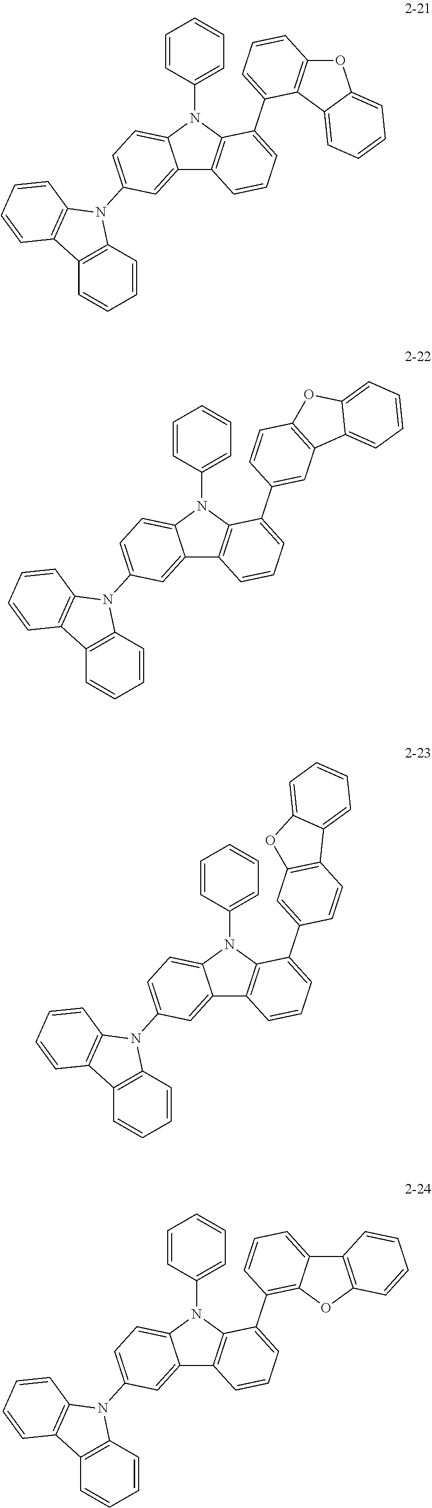 [SMILES: c1ccc(-n2c3ccc(-n4c5ccccc5c5ccccc54)cc3c3cccc(-c4cccc5oc6ccccc6c45)c32)cc1 ; c1ccc(-n2c3ccc(-n4c5ccccc5c5ccccc54)cc3c3cccc(-c4ccc5oc6ccccc6c5c4)c32)cc1 ; c1ccc(-n2c3ccc(-n4c5ccccc5c5ccccc54)cc3c3cccc(-c4ccc5c(c4)oc4ccccc45)c32)cc1 ; c1ccc(-n2c3ccc(-n4c5ccccc5c5ccccc54)cc3c3cccc(-c4cccc5c4oc4ccccc45)c32)cc1]

In another embodiments, the first host 344 and/or the second host 346 can comprise, but is not limited to, 9-(3-(9H-carbazol-9-yl)phenyl)-9H-carbazole-3-carbonitrile (mCP-CN), CBP, 3,3′-bis(N-carbazolyl)-1,1′-biphenyl (mCBP), 1,3-bis(carbazol-9-yl)benzene (mCP), bis[2-(diphenylphosphino)phenyl]ether oxide (DPEPO), 2,8-bis(diphenylphosphoryl)dibenzothiophene (PPT), 1,3,5-tri[(3-pyridyl)-phen-3-yl]benzene (TmPyPB), 2,6-di(9H-carbazol-9-yl)pyridine (PYD-2Cz), 2,8-di(9H-carbazol-9-yl)dibenzothiophene (DCzDBT), 3′,5′-di(carbazol-9-yl)-[1,1′-biphenyl]-3,5-dicarbonitrile (DCzTPA), 4′-(9H-carbazol-9-yl)biphenyl-3,5-dicarbonitrile (pCzB-2CN), 3′-(9H-carbazol-9-yl)biphenyl-3,5-dicarbonitrile (mCzB-2CN), diphenyl-4-triphenylsilyl-phenylphosphine oxide (TSPO1), 9-(9-phenyl-9H-carbazol-6-yl)-9H-carbazole (CCP), 4-(3-(triphenylen-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole, 9-(6-(9H-carbazol-9-yl)pyridin-3-yl)-9H-3,9′-bicabazole, 9,9′-diphenyl-9H,9′H-3,3′-bicarbazole (BCzPh), 1,3,5-tris(carbazole-9-yl)benzene (TCP), TCTA, 4,4′-Bis(carbazole-9-yl)-2,2′-dimethylbiphenyl (CDBP), 2,7-bis(carbazole-9-yl)-9,9-dimethylfluorene (DMFL-CBP), 2,2′,7,7′-tetrakis(carbazol-9-yl)-9,9-spirofluorene (Spiro-CBP), 3,6-bis(carbazole-9-yl)-9-(2-ethyl-hexyl)-9H-carbazole (TCzl) and/or combinations thereof.

In some embodiments, the contents of each of the host including the first host 344 and the second host 346 in the EML 340 can be about 50 wt % to about 99 wt %, for example, about 80 wt % to about 95 wt %, and the contents of the emitter 342 in the EML 340 can be about 1 wt % to about 50 wt %, for example, about 5 wt % to about 20 wt %, but is not limited thereto. When the EML 340 comprises both the first host 344 and the second host 346, the first host 344 and the second host 346 can be mixed, but is not limited to, with a weight ratio ranging from about 4:1 to about 1:4, for example from about 3:1 to about 1:3. As an example, the EML 340 can have a thickness of, but is not limited to, about 100 Å to about 500 Å.

The ETL 360 and the EIL 370 can be laminated sequentially between the EML 340 and the second electrode 220. An electron transporting material comprised in the ETL 360 has high electron mobility so as to provide electrons stably with the EML 340 by fast electron transportation.

In one example embodiment, electron transporting material in the ETL 360 can comprise at least one selected from the group consisting of an oxadiazole-based compound, a triazole-based compound, a phenanthroline-based compound, a benzoxazole-based compound, a benzothiazole-based compound, a benzimidazole-based compound and a triazine-based compound.

More particularly, the electron transporting material in the ETL 360 can include, but is not limited to, at least one selected from the group consisting of tris-(8-hydroxyquinoline aluminum (Alq₃), 2-biphenyl-4-yl-5-(4-t-butylphenyl)-1,3,4-oxadiazole (PBD), spiro-PBD, lithium quinolate (Liq), 1,3,5-tris(N-phenylbenzimidazol-2-yl)benzene (TPBi), bis(2-methyl-8-quinolinolato-N1,08)-(1,1′-biphenyl-4-olato)aluminum (BAlq), 4,7-diphenyl-1,10-phenanthroline (Bphen), 2,9-bis(naphthalene-2-yl)4,7-diphenyl-1,10-phenanthroline (NBphen), 2,9-dimethyl-4,7-diphenyl-1,10-phenathroline (BCP), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), 1,3,5-tri(p-pyrid-3-yl-phenyl)benzene (TpPyPB), 2,4,6-tris(3′-(pyridin-3-yl)biphenyl-3-yl)1,3,5-triazine (TmPPPyTz), poly[9,9-bis(3′-((N,N-dimethyl)-N-ethylammonium)-propyl)-2,7-fluorene]-alt-2,7-(9,9-dioctylfluorene)] (PFNBr), tris(phenylquinoxaline (TPQ), TSPO1, 2-[4-(9,10-di-2-naphthalen2-yl-2-anthracen-2-yl)phenyl]-1-phenyl-1H-benzimidazole (ZADN), and/or combinations thereof.

The EIL 370 is disposed between the second electrode 220 and the ETL 360, and can improve physical properties of the second electrode 220 and therefore, can enhance the lifespan of the OLED D1. In some embodiments, electron injecting material in the EIL 370 can comprise, but is not limited to, an alkali metal halide or an alkaline earth metal halide such as LiF, CsF, NaF, BaF₂ and the like, and/or an organometallic compound such as Liq, lithium benzoate, sodium stearate, and the like. Alternatively, the EIL 370 can be omitted.

When holes are transferred to the second electrode 220 via the EML 340 and/or electrons are transferred to the first electrode 210 via the EML 340, the OLED D1 can have short lifespan and reduced luminous efficiency. In order to prevent those phenomena, the OLED D1 in accordance with this aspect of the present disclosure can have at least one exciton blocking layer adjacent to the EML 340.

As an example, the OLED D1 can comprise the EBL 330 disposed between the HTL 320 and the EML 340 so as to control and prevent electron transfers. In some embodiments, an electron blocking material in the EBL 330 can comprise, but is not limited to, at least one selected from the group consisting of TCTA, Tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, 1,3-Bis(carbazol-9-yl)benzene (mCP), 3,3-Di(9H-carbazol-9-yl)biphenyl (mCBP), CuPc, DNTPD, TDAPB, DCDPA, 2,8-bis(9-phenyl-9H-carbazol-3-yl)dibenzo[b,d]thiophene and/or combinations thereof.

In addition, the OLED D1 can further comprise the HBL 350 as a second exciton blocking layer between the EML 340 and the ETL 360 so that holes cannot be transferred from the EML 340 to the ETL 360. In some embodiments, a hole blocking material in the HBL 350 can comprise, but is not limited to, at least one selected from the group consisting of an oxadiazole-based compound, a triazole-based compound, a phenanthroline-based compound, a benzoxazole-based compound, a benzothiazole-based compound, a benzimidazole-based compound, and a triazine-based compound.

For example, the hole blocking material in the HBL 350 can comprise material having a relatively low HOMO energy level compared to the luminescent materials in EML 340. The HBL 350 can comprise, but is not limited to, at least one selected from the group consisting of BCP, BAlq, Alq₃, PBD, spiro-PBD, Liq, bis-4,5-(3,5-di-3-pyridylphenyl)-2-methylpyrimidine (B3PYMPM), DPEPO, 9-(6-(9H-carbazol-9-yl)pyridine-3-yl)-9H-3,9′-bicarbazole, TSPO1 and/or combinations thereof.

As described above, the EML 340 comprises the emitter 342 and the host 344 and/or 346, and the emitter 342 can comprise at least one of the organic compounds having the structure of Chemical Formulae 1 to 3. The organic compound having the structure of Chemical Formulae 1 to 3 may be fluorescent emitter with a wide plate-like structure. The singlet exciton energy of the first host 344 and/or the second host 346 can be transferred efficiently to the emitter 342 by FRET mechanism. Accordingly, the luminous efficiency and the luminous lifetime of the OLED D1 can be improved.

The organic light emitting device and the OLED D1 with a single emitting part are shown in FIG. 2. In another example embodiments, an organic light emitting display device can implement full-color including white color.

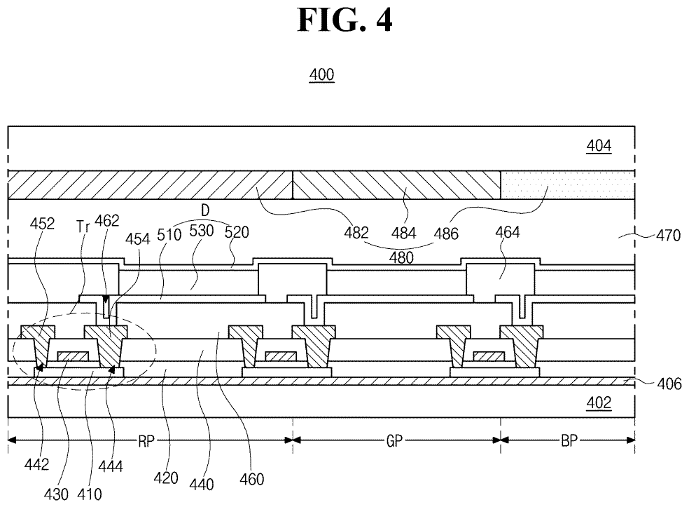

FIG. 4 illustrates a schematic cross-sectional view of an organic light emitting display device in accordance with another example embodiments of the present disclosure. As illustrated in FIG. 4, the organic light emitting display device 400 comprises a first substrate 402 that defines each of a red pixel region RP, a green pixel region GP and a blue pixel region BP, a second substrate 404 facing the first substrate 402, a thin film transistor Tr on the first substrate 402, an OLED D disposed between the first and second substrates 402 and 404 and emitting white (W) light and a color filter layer 480 disposed between the OLED D and the second substrate 404.

Each of the first and second substrates 402 and 404 can comprise, but is not limited to, glass, a flexible material and/or polymer plastics. For example, each of the first and second substrates 402 and 404 can include at least one selected from the group consisting of PI, PES, PEN, PET, PC and combinations thereof. In certain embodiments, the second substrate 404 can be omitted. The first substrate 402, on which a thin film transistor Tr and the OLED D are arranged, forms an array substrate.

A buffer layer 406 can be disposed on the first substrate 402. The thin film transistor Tr is disposed on the buffer layer 406 correspondingly to each of the red pixel region (RP), the green pixel region (GP) and the blue pixel region (BP). In certain embodiments, the buffer layer 406 can be omitted.

A semiconductor layer 410 is disposed on the buffer layer 406. The semiconductor layer 410 can be made of or comprise oxide semiconductor material or polycrystalline silicon.

A gate insulating layer 420 comprising an insulating material, for example, inorganic insulating material such as silicon oxide (SiOₓ, wherein 0<x≤2) or silicon nitride (SiNₓ, wherein 0<x≤2) is disposed on the semiconductor layer 410.

A gate electrode 430 made of a conductive material such as a metal is disposed over the gate insulating layer 420 so as to correspond to a center of the semiconductor layer 410. An interlayer insulating layer 440 including an insulating material, for example, inorganic insulating material such as SiOₓ or SiNₓ (wherein 0<x≤2), or an organic insulating material such as benzocyclobutene or a photo-acryl, is disposed on the gate electrode 430.

The interlayer insulating layer 440 has a first semiconductor layer contact hole 442 and a second semiconductor layer contact hole 444 that expose or do not cover a portion of the surface nearer to the opposing ends than to a center of the semiconductor layer 410. The first and second semiconductor layer contact holes 442 and 444 are disposed on opposite sides of the gate electrode 430 with spacing apart from the gate electrode 430.

A source electrode 452 and a drain electrode 454, which are made of or comprise a conductive material such as a metal, are disposed on the interlayer insulating layer 440. The source electrode 452 and the drain electrode 454 are spaced apart from each other with respect to the gate electrode 430. The source electrode 452 and the drain electrode 454 contact both sides of the semiconductor layer 410 through the first and second semiconductor layer contact holes 442 and 444, respectively.

The semiconductor layer 410, the gate electrode 430, the source electrode 452 and the drain electrode 454 constitute the thin film transistor Tr, which acts as a driving element.

Although not shown in FIG. 4, the gate line GL and the data line DL, which cross each other to define the pixel region P, and a switching element Ts, which is connected to the gate line GL and the data line DL, can be further formed in the pixel region P. The switching element Ts is connected to the thin film transistor Tr, which is a driving element. In addition, the power line PL is spaced apart in parallel from the gate line GL or the data line DL, and the thin film transistor Tr can further comprise the storage capacitor Cst configured to constantly keep a voltage of the gate electrode 430 for one frame.

A passivation layer 460 is disposed on the source electrode 452 and the drain electrode 454 and covers the thin film transistor Tr over the entire first substrate 402. The passivation layer 460 has a drain contact hole 462 that exposes or does not cover the drain electrode 454 of the thin film transistor Tr.

The OLED D is located on the passivation layer 460. The OLED D comprises a first electrode 510 that is connected to the drain electrode 454 of the thin film transistor Tr, a second electrode 520 facing the first electrode 510 and an emissive layer 530 disposed between the first and second electrodes 510 and 520.

The first electrode 510 formed for each pixel region RP, GP or BP can be an anode and can comprise a conductive material having relatively high work function value. For example, the first electrode 510 can comprise, but is not limited to, at least one selected from the group consisting of ITO, IZO, ITZO, SnO, ZnO, ICO, AZO, and combinations thereof. Alternatively, a reflective electrode or a reflective layer can be disposed under the first electrode 510. For example, the reflective electrode or the reflective layer can comprise, but is not limited to, Ag or an APC alloy (Ag-alloy).

A bank layer 464 is disposed on the passivation layer 460 in order to cover edges of the first electrode 510. The bank layer 464 exposes or does not cover a center of the first electrode 510 corresponding to each of the red pixel region (RP), the green pixel region (GP) and the blue pixel region (BP). In certain embodiments, the bank layer 464 can be omitted.

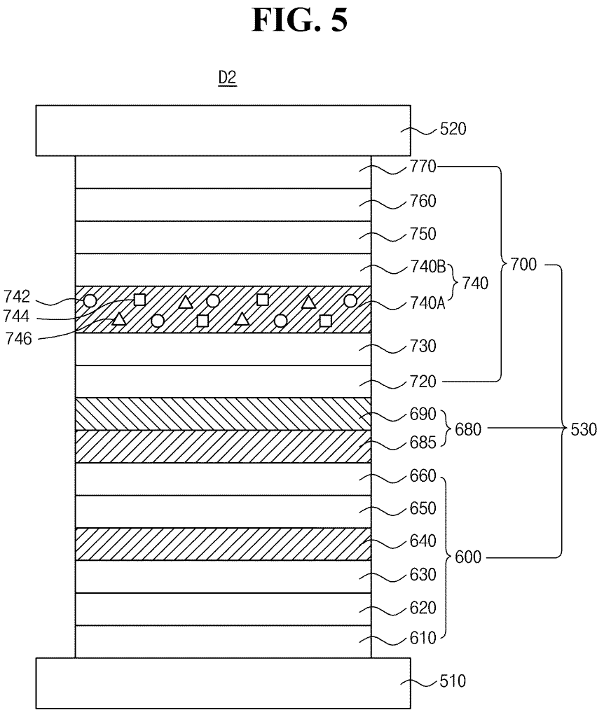

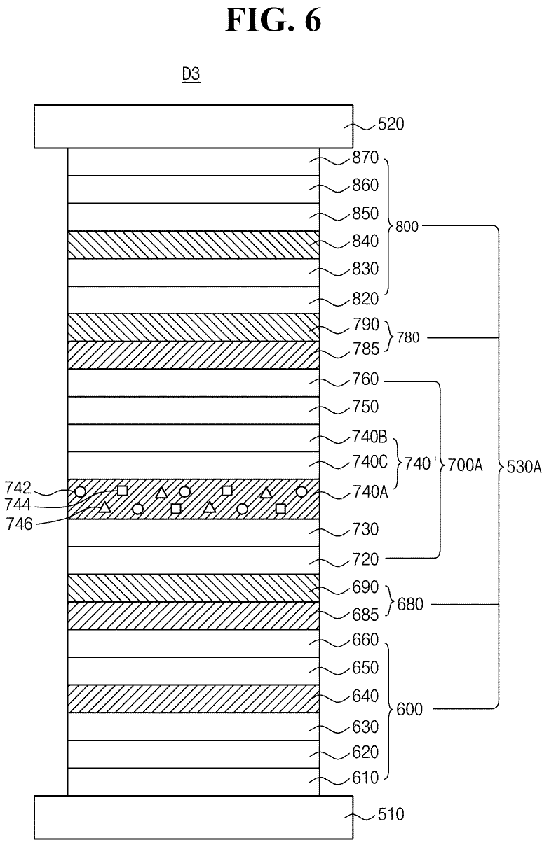

An emissive layer 530 that can comprise multiple emitting parts is disposed on the first electrode 510. As illustrated in FIGS. 5 and 6, the emissive layer 530 can comprise multiple emitting parts 600, 700, 700A, and 800 and at least one charge generation layer 680 and 780. Each of the emitting parts 600, 700, 700A and 800 comprises at least one emitting material layer (EML) and can further comprise a hole injection layer (HIL), a hole transport layer (HTL), an electron blocking layer (EBL), a hole blocking layer (HBL), an electron transport layer (ETL) and/or an electron injection layer (EIL).

The second electrode 520 can be disposed on the first substrate 402 above which the emissive layer 530 can be disposed. The second electrode 520 can be disposed over an entire display area, can comprise a conductive material with a relatively low work function value compared to the first electrode 510, and can be a cathode. For example, the second electrode 520 can comprise, but is not limited to, at least one selected from the group consisting of Al, Mg, Ca, Ag, alloy thereof, and combinations thereof such as Al—Mg.

Since the light emitted from the emissive layer 530 is incident to the color filter layer 480 through the second electrode 520 in the organic light emitting display device 400 in accordance with the second embodiment of the present disclosure, the second electrode 520 has a thin thickness so that the light can be transmitted.

The color filter layer 480 is disposed on the OLED D and comprises a red color filter pattern 482, a green color filter pattern 484 and a blue color filter pattern 486 each of which is disposed correspondingly to the red pixel region (RP), the green pixel region (GP) and the blue pixel region (BP), respectively. Although not shown in FIG. 4, the color filter layer 480 can be attached to the OLED D through an adhesive layer. Alternatively, the color filter layer 480 can be disposed directly on the OLED D.

In addition, an encapsulation film 470 can be disposed on the second electrode 520 in order to prevent or reduce outer moisture from penetrating into the OLED D. The encapsulation film 470 can have, but is not limited to, a laminated structure including a first inorganic insulating film, an organic insulating film and a second inorganic insulating film (170 in FIG. 2). In addition, a polarizing plate can be attached onto the second substrate 404 to reduce reflection of external light. For example, the polarizing plate can be a circular polarizing plate.

In FIG. 4, the light emitted from the OLED D is transmitted through the second electrode 520 and the color filter layer 480 is disposed on the OLED D. In this case, the organic light emitting display device 400 can be a top-emission type. Alternatively, when the organic light emitting display device 400 is a bottom-emission type, the light emitted from the OLED D is transmitted through the first electrode 510 and the color filter layer 480 can be disposed between the OLED D and the first substrate 402.

In addition, a color conversion layer may be formed or disposed between the OLED D and the color filter layer 480. The color conversion layer may comprise a red color conversion layer, a green color conversion layer and a blue color conversion layer each of which is disposed correspondingly to each pixel region (RP, GP and BP), respectively, so as to convert the white (W) color light to each of a red, green and blue color lights, respectively. Alternatively, the organic light emitting display device 400 can comprise the color conversion layer instead of the color filter layer 480.

As described above, the white (W) color light emitted from the OLED D is transmitted through the red color filter pattern 482, the green color filter pattern 484 and the blue color filter pattern 486 each of which is disposed correspondingly to the red pixel region (RP), the green pixel region (GP) and the blue pixel region (BP), respectively, so that red, green and blue color lights are displayed in the red pixel region (RP), the green pixel region (GP) and the blue pixel region (BP).

An OLED that can be applied into the organic light emitting display device 400 will be described in more detail. FIG. 5 illustrates a schematic cross-sectional view of an organic light emitting diode having a tandem structure of two emitting parts.

As illustrated in FIG. 5, the OLED D2 in accordance with some embodiments of the present disclosure comprises a first electrode 510, a second electrode 520 facing to the first electrode 510, and an emissive layer 530 disposed between the first and second electrodes 510 and 520. The emissive layer 530 comprises a first emitting part 600 disposed between the first electrode 510 and the second electrode 520, a second emitting part 700 disposed between the first emitting part 600 and the second electrode 520 and a charge generation layer (CGL) 680 disposed between the first emitting part 600 and the second emitting part 700.

The first electrode 510 can be an anode and can comprise a conductive material having relatively high work function value such as TCO. For example, the first electrode 510 can comprise, but is not limited to, at least one selected from the group consisting of ITO, IZO, ITZO, SnO, ZnO, ICO, AZO, and combinations thereof. The second electrode 520 can be a cathode and can comprise a conductive material with a relatively low work function value. For example, the second electrode 520 can comprise, but is not limited to, a highly reflective material such as Al, Mg, Ca, Ag, alloy thereof and/or combinations thereof such as Al—Mg.

The first emitting part 600 comprises a first emitting material layer (EML1) 640. The first emitting part 600 can further comprise at least one of a hole injection layer (HIL) 610 disposed between the first electrode 510 and the EML1 640, a first hole transport layer (HTL1) 620 disposed between the HIL 610 and the EML1 640, and a first electron transport layer (ETL1) 660 disposed between the EML1 640 and the CGL 680. Alternatively, the first emitting part 600 can further comprise a first electron blocking layer (EBL1) 630 disposed between the HTL1 620 and the EML1 640 and/or a first hole blocking layer (HBL1) 650 disposed between the EML1 640 and the ETL1 660.

The second emitting part 700 comprises a second emitting material layer (EML2) 740. The second emitting part 700 can further comprise at least one of a second hole transport layer (HTL2) 720 disposed between the CGL 680 and the EML2 740, a second electron transport layer (ETL2) 760 disposed between the second electrode 520 and the EML2 740 and an electron injection layer (EIL) 770 disposed between the second electrode 520 and the ETL2 760. Alternatively, the second emitting part 700 can further comprise a second electron blocking layer (EBL2) 730 disposed between the HTL2 720 and the EML2 740 and/or a second hole blocking layer (HBL2) 750 disposed between the EML2 740 and the ETL2 760.

At least one of the EML1 640 or the EML2 740 can comprise at least one of the organic compounds having the structure of Chemical Formulae 1 to 3 so that it can emit red to green color light, and the other of the EML1 640 and the EML2 740 can emit blue color light, so that the OLED D2 can realize white (W) emission. Hereinafter, the OLED D2 where the EML2 740 comprises at least one of the organic compounds having the structure of Chemical Formulae 1 to 3 to emit red to green color light will be described in detail.

The HIL 610 is disposed between the first electrode 510 and the HTL1 620 and may improve an interface property between the inorganic first electrode 510 and the organic HTL1 620. In some embodiments, the hole injecting material in the HIL 610 can comprise, but is not limited to, at least one selected from the group consisting of MTDATA, NATA, 1T-NATA, 2T-NATA, CuPc, TCTA, NPB (NPD), DNTPD, HAT-CN, F4-TCNQ, F6-TCNNQ, TDAPB, PEDOT/PSS, N-(biphenyl-4-yl)-9,9-dimethyl-N-(4-(9-phenyl-9H-carbazol-3-yl)phenyl)-9H-fluoren-2-amine, NPNPB and combinations thereof. In another embodiments, the HIL 610 can comprise hole transporting material doped with hole injecting material. In certain embodiments, the HIL 610 can be omitted in compliance of the OLED D2 property.

In some embodiments, each of hole transporting materials in the HTL1 620 and the HTL2 720 can independently comprise, but is not limited to, at least one selected from the group consisting of TPD, NPB (NPD), DNTPD, CBP, poly-TPD, TFB, TAPC, DCDPA, N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine, N-([1,1′-Biphenyl]-4-yl)-9,9-dimethyl-N-(4-(9-phenyl-9H-carbazol-3-yl)phenyl)-9H-fluoren-2-amine and combinations thereof.

Each of the ETL1 660 and the ETL2 760 facilitates electron transportation in each of the first emitting part 600 and the second emitting part 700, respectively. As an example, each of electron transporting materials in the ETL1 660 and the ETL2 760 can independently comprise at least one selected from the group consisting of an oxadiazole-based compound, a triazole-based compound, a phenanthroline-based compound, a benzoxazole-based compound, a benzothiazole-based compound, a benzimidazole-based compound and a triazine-based compound. For example, each of electron transporting materials in the ETL1 660 and the ETL2 760 can independently comprise, but is not limited to, at least one selected from the group consisting of Alq₃, PBD, spiro-PBD, Liq, TPBi, BAlq, Bphen, NBphen, BCP, TAZ, NTAZ, TpPyPB, TmPPPyTz, PFNBr, TPQ, TSPO1, ZADN and combinations thereof.

The EIL 770 is disposed between the second electrode 520 and the ETL2 760, and can improve physical properties of the second electrode 520 and therefore, can enhance the lifespan of the OLED D2. In some embodiments, electron injecting material in the EIL 770 can comprise, but is not limited to, at least one selected from the group consisting of an alkali metal halide or an alkaline earth metal halide such as LiF, CsF, NaF, BaF₂ and the like, and an organometallic compound such as Liq, lithium benzoate, sodium stearate, and the like. In certain embodiments, the EIL 770 can be omitted.

Each of electron blocking materials in the EBL1 630 and the EBL2 730 can independently comprise, but is not limited to, at least one selected from the group consisting of TCTA, tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, mCP, mCBP, CuPc, DNTPD, TDAPB, DCDPA, 2,8-bis(9-phenyl-9H-carbazol-3-yl)dibenzo[b,d]thiophene and combinations thereof, respectively.

Each of hole blocking materials in the HBL1 650 and the HBL2 750 can independently comprise, but is not limited to, at least one selected from the group consisting of an oxadiazole-based compound, a triazole-based compound, a phenanthroline-based compound, a benzoxazole-based compound, a benzothiazole-based compound, a benzimidazole-based compound, and a triazine-based compound. For example, each of the hole blocking materials in the HBL1 650 and the HBL2 750 can independently comprise, but is not limited to, at least one selected from the group consisting of BCP, BA1q, Alq₃, PBD, spiro-PBD, Liq, B3PYMPM, DPEPO, 9-(6-(9H-carbazol-9-yl)pyridine-3-yl)-9H-3,9′-bicarbazole, TSPO1 and combinations thereof, respectively.

The CGL 680 is disposed between the first emitting part 600 and the second emitting part 700. The CGL 680 comprises an N-type charge generation layer (N-CGL) 685 disposed between the ETL1 660 and the HTL2 720 and a P-type charge generation layer (P-CGL) 690 disposed between the N-CGL 685 and the HTL2 720. The N-CGL 685 injects electrons to the EML1 640 of the first emitting part 600 and the P-CGL 690 injects holes to the EML2 740 of the second emitting part 700.

The N-CGL 685 can be an organic layer including electron transporting material doped with an alkali metal such as Li, Na, K and Cs and/or an alkaline earth metal such as Mg, Sr, Ba and Ra. For example, the contents of the alkali metal or the alkaline earth metal in the N-CGL 685 can be, but is not limited to, between about 0.01 wt % and about 30 wt %, between about 0.05 wt % and about 20 wt %, or between about 1 wt % and about 10 wt %.

The P-CGL 690 can comprise, but is not limited to, at least one inorganic material selected from the group consisting of tungsten oxide (WOₓ), molybdenum oxide (MoOₓ), beryllium oxide (Be₂O₃), vanadium oxide (V₂O₅) and combinations thereof. In another example embodiment, the P-CGL 690 can comprise a hole transporting material doped with a hole injecting material (e.g., HAT-CN, F4-TCNQ and/or F6-TCNNQ). The contents of the hole injecting material in the P-CGL 690 can be, but is not limited to, between about 2 wt % and about 15 wt %, or between about 5 wt % and about 10 wt %.

The EML1 640 can be a blue emitting material layer (B-EML). In this case, the EML1 640 can be a blue emitting material layer, a sky-blue emitting material layer or a deep-blue emitting material layer. The EML1 640 can comprise a blue host and blue emitter (dopant).

The blue host can comprise at least one of a P-type blue host or an N-type blue host. For example, the blue host can comprise, but is not limited to, at least one selected from the group consisting of mCP, 9-(3-(9H-carbazol-9-yl)phenyl)-9H-carbazole-3-carbonitrile (mCP-CN), mCBP, CBP-CN, 9-(3-(9H-carbazol-9-yl)phenyl)-3-(diphenylphosphoryl)-9H-carbazole (mCPPO1) 3,5-di(9H-carbazol-9-yl)biphenyl (Ph-mCP), TSPO1, 9-(3′-(9H-carbazol-9-yl)-[1,1′-biphenyl]-3-yl)-9H-pyrido[2,3-b]indole (CzBPCb), bis(2-methylphenyl)diphenylsilane (UGH-1), 1,4-bis(triphenylsilyl)benzene (UGH-2), 1,3-Bis(triphenylsilyl)benzene (UGH-3), 9,9-spirobifluoren-2-yl-diphenyl-phosphine oxide (SPPO1), 9,9′-(5-(triphenylsilyl)-1,3-phenylene)bis(9H-carbazole) (SimCP) and combinations thereof.

The blue emitter can comprise at least one selected from the group consisting of blue phosphorescent material, blue fluorescent material and blue delayed fluorescent material. As an example, the blue emitter can comprise, but is not limited to, perylene, 4,4′-bis[4-(di-p-tolylamino)styryl]biphenyl (DPAVBi), 4-(di-p-tolylamino)-4-4′-[(di-p-tolylamino)styryl]stilbene (DPAVB), 4,4′-bis[4-(diphenylamino)styryl]biphenyl (BDAVBi), 2,7-bis(4-diphenylamino)styryl)-9,9-spirofluorene (spiro-DPVBi), [1,4-bis[2-[4-[N,N-di(p-tolyl)amino]phenyl]vinyl]benzene (DSB), 1-4-di-[4-(N,N-diphenyl)amino]styryl-benzene (DSA), 2,5,8,11-tetra-tert-butylperylene (TBPe), bis(2-hydroxylphenyl)-pyridine)beryllium (Bepp₂), 9-(9-phenylcarbazole-3-yl)-10-(naphthalene-1-yl)anthracene (PCAN), mer-tris(1-phenyl-3-methylimidazolin-2-ylidene-C,C(2)′iridium(III) (mer-Ir(pmi)₃), fac-tris(1,3-diphenyl-benzimidazolin-2-ylidene-C,C(2)′iridium(III) (fac-Ir(dpbic)₃), bis(3,4,5-trifluoro-2-(2-pyridyl)phenyl-(2-carboxypyridyl)iridium(III) (Ir(tfpd)₂pic), tris(2-(4,6-difluorophenyl)pyridine))iridium(III) (Ir(Fppy)₃), bis[2-(4,6-difluorophenyl)pyridinato-C²,N](picolinato)iridium(III) (FIrpic) and combinations thereof.

The contents of the blue host in the EMIL1 640 can be about 50 wt % to about 99 wt %, for example, about 80 wt % to about 95 wt %, and the contents of the blue emitter in the EML1 640 can be about 1 wt % to about 50 wt %, for example, about 5 wt % to about 20 wt %, but is not limited thereto. When the EML1 640 comprises both the P-type blue host and the N-type blue host, the P-type blue host and the N-type blue host can be admixed, but is not limited to, with a weight ratio of about 4:1 to about 1:4, for example about 3:1 to about 1:3.

The EML2 740 can comprise a first layer (a lower emitting material layer) 740A disposed between the EBL2 730 and the HBL2 750 and a second layer (an upper emitting material layer) 740B disposed between the first layer 740A and the HBL2 750. One of the first layer 740A or the second layer 740B can emit red to yellow color light and the other of the first layer 740A and the second layer 740B can emit green color light. Hereinafter, the EML2 740 where the first layer 740A emits a red to yellow color light and the second layer 740B emits a green color light will be described in detail.

The first layer 740A comprises an emitter 742, a first host 744, and optionally, a second host 746. The emitter 742 may be a fluorescent emitter (fluorescent dopant) comprising a compound having at least one of the structures of Chemical Formulae 1 to 3, and can emit red to yellow color light.

The first host 744 can be a P-type host that can comprise a carbazole-based organic compound, an aryl or hetero aryl amine-based organic compound with at least one fused aromatic or hetero aromatic moiety and/or an aryl or hetero aryl amine-based organic compound having a spirofluorene moiety. The second host 746 can be an N-type host that can comprise an azine-based organic compound and a quinazoline-based organic compound.

In some embodiments, the first host 744 can comprise, but is not limited to, at least one of the compounds having the structures of Chemical Formulae 4 to 6. In another embodiment, the first host 744 and/or the second host 746 can comprise, but is not limited to, at least one selected from the group consisting of mCP-CN, CBP, mCBP, mCP, DPEPO, PPT, TmPyPB, PYD-2Cz, DCzDBT, DCzTPA, pCzB-2CN, mCzB-2CN, TSPO1, CCP, 4-(3-(triphenylen-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole, 9-(6-(9H-carbazol-9-yl)pyridin-3-yl)-9H-3,9′-bicabazole, BCzPh, TCP, TCTA, CDBP, DMFL-CBP, Spiro-CBP, TCzl and combinations thereof.

The contents of the emitter 742, the first host 744, and the second host 746 can be identical as the corresponding materials described with referring to FIG. 3.

The second layer 740B can comprise a green host and a green emitter (green dopant). The green host can comprise at least one of a P-type green host or an N-type green host. In some embodiments, the green host can be identical to the first host 744 and/or the second host 746. In another example embodiments, the green host can comprise, but is not limited to, at least one selected from the group consisting of mCP-CN, CBP, mCBP, mCP, DPEPO, PPT, TmPyPB, PYD-2Cz, DCzDBT, DCzTPA, pCzB-2CN, mCzB-2CN, TSPO1, CCP, 4-(3-(triphenylen-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole, 9-(6-(9H-carbazol-9-yl)pyridin-3-yl)-9H-3,9′-bicabazole, BCzPh, BCZ, TCP, TCTA, CDBP, DMFL-CBP, Spiro-CBP, TCzl and combinations thereof.

The green emitter can comprise at least one of green phosphorescent material, green fluorescent material or green delayed fluorescent material. As an example, the green emitter can comprise, but is not limited to, at least one selected from the group consisting of [bis(2-phenylpyridine)](pyridyl-2-benzofuro[2,3-b]pyridine)iridium, tris[2-phenylpyridine]iridium(III) (Ir(ppy)₃), fac-tris(2-phenylpyridine)iridium(III) (fac-Ir(ppy)₃), bis(2-phenylpyridine)(acetylacetonate)iridium(III) (Ir(ppy)₂(acac)), tris[2-(p-tolyl)pyridine]iridium(III) (Ir(mppy)₃), bis(2-(naphthalen-2-yl)pyridine)(acetylacetonate)iridium(III) (Ir(npy)₂acac), tris(2-phenyl-3-methyl-pyridine)iridium (Ir(3mppy)₃), fac-tris(2-(3-p-xylyl)phenyl)pyridine iridium(III) (TEG) and combinations thereof.

The contents of the green host in the second layer 740B can be about 50 wt % to about 99 wt %, for example, about 80 wt % to about 95 wt %, and the contents of the green emitter in the second layer 740B can be about 1 wt % to about 50 wt %, for example, about 5 wt % to about 20 wt %, but is not limited thereto. When the second layer 740B comprises both the P-type green host and the N-type green host, the P-type green host and the N-type green host can be admixed, but is not limited to, with a weight ratio of about 4:1 to about 1:4, for example about 3:1 to about 1:3.

Alternatively or additionally, the EML2 740 can further comprise a third layer (740C in FIG. 6) that can emit yellow-green color light and can be disposed between the first layer 740A of the red EML and the second layer 740B of the green EML.

The OLED D2 with a tandem structure in accordance with this embodiment comprises at least one of the organic compounds having the structure of Chemical Formulae 1 to 3. The at least one of the organic compounds having the structure of Chemical Formulae 1 to 3 may have a wide plate-like structure and can receive singlet exciton energy from the host 744 and/or 746. The luminous efficiency and the luminous lifespan of the OLED D2 can be improved.

An OLED can have three or more emitting parts to form a tandem structure. FIG. 6 is a schematic cross-sectional view illustrating an organic light emitting diode in accordance with yet another example embodiments of the present disclosure.

As illustrated in FIG. 6, the OLED D4 comprises first and second electrodes 510 and 520 facing each other and an emissive layer 530A disposed between the first and second electrodes 510 and 520. The emissive layer 530A comprises a first emitting part 600 disposed between the first and second electrodes 510 and 520, a second emitting part 700A disposed between the first emitting part 600 and the second electrode 520, a third emitting part 800 disposed between the second emitting part 700A and the second electrode 520, a first charge generation layer (CGL1) 680 disposed between the first and second emitting parts 600 and 700A, and a second charge generation layer (CGL2) 780 disposed between the second and third emitting parts 700A and 800.

The first emitting part 600 comprises a first emitting material layer (EML1) 640. The first emitting part 600 can further comprise at least one of a hole injection layer (HIL) 610 disposed between the first electrode 510 and the EML1 640, a first hole transport layer (HTL1) 620 disposed between the HIL 610 and the EML1 640, or a first electron transport layer (ETL1) 660 disposed between the EML1 640 and the CGL1 680. Alternatively, the first emitting part 600 can further comprise a first electron blocking layer (EBL1) 630 disposed between the HTL1 620 and the EML1 640 and/or a first hole blocking layer (HBL1) 650 disposed between the EML1 640 and the ETL1 660.

The second emitting part 700A comprises a second emitting material layer (EML2) 740′. The second emitting part 700A can further comprise at least one of a second hole transport layer (HTL2) 720 disposed between the CGL1 680 and the EML2 740′ or a second electron transport layer (ETL2) 760 disposed between the EML2 740′ and the CGL2 780. Alternatively, the second emitting part 700A can further comprise a second electron blocking layer (EBL2) 730 disposed between the HTL2 720 and the EML2 740′ and/or a second hole blocking layer (HBL2) 750 disposed between the EML2 740′ and the ETL2 760.

The third emitting part 800 comprises a third emitting material layer (EML3) 840. The third emitting part 800 can further comprise at least one of a third hole transport layer (HTL3) 820 disposed between the CGL2 780 and the EML3 840, a third electron transport layer (ETL3) 860 disposed between the second electrode 520 and the EML3 840, or an electron injection layer (EIL) 870 disposed between the second electrode 520 and the ETL3 860. Alternatively, the third emitting part 800 can further comprise a third electron blocking layer (EBL3) 830 disposed between the HTL3 820 and the EML3 840 and/or a third hole blocking layer (HBL3) 850 disposed between the EML3 840 and the ETL3 860.

The CGL1 680 is disposed between the first emitting part 600 and the second emitting part 700A, and the CGL2 780 is disposed between the second emitting part 700A and the third emitting part 800. The CGL1 680 comprises a first N-type charge generation layer (N-CGL1) 685 disposed between the ETL1 660 and the HTL2 720 and a first P-type charge generation layer (P-CGL1) 690 disposed between the N-CGL1 685 and the HTL2 720. The CGL2 780 comprises a second N-type charge generation layer (N-CGL2) 785 disposed between the ETL2 760 and the HTL3 820 and a second P-type charge generation layer (P-CGL2) 790 disposed between the N-CGL2 785 and the HTL3 820. Each of the N-CGL1 685 and the N-CGL2 785 injects electrons to the EML1 640 of the first emitting part 600 and the EML2 740′ of the second emitting part 700A, respectively, and each of the P-CGL1 690 and the P-CGL2 790 injects holes to the EML2 740′ of the second emitting part 700A and the EML3 840 of the third emitting part 800, respectively.

The materials comprised in the HIL 610, the HTL1 to the HTL3 620, 720 and 820, the EBL1 to the EBL3 630, 730 and 830, the HBL1 to the HBL3 650, 750 and 850, the ETL1 to the ETL3 660, 760 and 860, the EIL 870, the CGL1 680, and the CGL2 780 can be identical to the materials with referring to FIGS. 3 and 5.

At least one of the EML1 640, the EML2 740′ or the EML3 840 can comprise at least one of the organic compounds having the structure of Chemical Formulae 1 to 3. For example, at least one of the EML1 640, the EML2 740′ and the EML3 840 can emit red to green color light, and the other of the EML1 640, the EML2 740′ and the EML3 840 can emit a blue color light so that the OLED D4 can realize white (W) emission. Hereinafter, the OLED D4 where the EML2 740′ comprises at least one of the compounds having the structure of Chemical Formulae 1 to 3 and emits red to green color light, and each of the EML1 640 and the EML3 840 emits a blue color light will be described in detail.

Each of the EML1 640 and the EML3 840 can be independently a blue emitting material layer (B-EML). In this case, each of the EML1 640 and the EML3 840 can be independently a blue emitting material layer, a sky-blue emitting material layer or a deep-blue emitting material layer. Each of the EML1 640 and the EML3 840 can independently comprises a blue host and a blue emitter (dopant). Each of the blue host and the blue emitter can be identical to corresponding materials with referring to FIG. 5. For example, the blue emitter can comprise at least one of a blue phosphorescent material, a blue fluorescent material or a blue delayed fluorescent material. Alternatively, the blue emitter in the EML1 640 can be identical to or different from the blue emitter in the EML3 840 in terms of color and/or luminous efficiency.

The EML2 740′ can comprise a first layer (lower emitting material layer) 740A disposed between the EBL2 730 and the HBL2 750, a second layer (upper emitting material layer) 740B disposed between the first layer 740A and the HBL2 750, and a third layer (middle emitting material layer) 740C disposed between the first layer 740A and the second layer 740B. One of the first layer 740A and the second layer 740B can emit red to yellow color and the other of the first layer 740A and the second layer 740B can emit green color. Hereinafter, the EML2 740′ where the first layer 740A emits a red to yellow color and the second layer 740B emits a green color will be described in detail.

The first layer 740A can comprise an emitter 742, a first host 744, and optionally, a second host 746. The emitter 742 is fluorescent emitter (fluorescent dopant) at least one of the compounds having the structure of Chemical Formulae 1 to 3, and can emit red to yellow color light.

The first host 744 can be a P-type host that can comprise a carbazole-based organic compound, an aryl or hetero aryl amine-based organic compound with at least one fused aromatic or hetero aromatic moiety and/or an aryl or hetero aryl amine-based organic compound having a spirofluorene moiety. The second host 746 can be an N-type host that can comprise an azine-based organic compound and a quinazoline-based organic compound.

The first host 744 and/or the second host 746 can be identical to the corresponding materials with referring to FIGS. 3 and 5. The contents of the emitter 742, the first host 744, and the second host 746 can be identical as the corresponding materials described with referring to FIG. 3.

The second layer 740B can comprise a green host and green emitter (green dopant). The kinds and the contents of the green host and the green emitter can be identical as the corresponding materials described with referring to FIG. 5. For example, the green emitter can comprise at least one of green phosphorescent material, green fluorescent material and green delayed fluorescent material.

The third layer 740C can be a yellow-green emitting material layer. The third layer 740C can comprise a yellow-green host and a yellow-green emitter (dopant). The yellow-green host can comprise at least one of a P-type yellow-green host or an N-type yellow-green host. As an example, the yellow-green host can be identical to the first host 744 and/or the second host 746.

The yellow-green emitter can comprise at least one of yellow-green fluorescent material, yellow-green phosphorescent material or yellow-green delayed fluorescent material. For example, the yellow-green emitter can comprise, but is not limited to, at least one selected from the group consisting of 5,6,11,12-tetraphenylnaphthalene (Rubrene), 2,8-di-tert-butyl-5,11-bis(4-tert-butylphenyl)-6,12-diphenyltetracene (TBRb), bis(2-phenylbenzothiazolato)(acetylacetonate)iridium(III) (Ir(BT)₂(acac)), bis(2-(9,9-diethytl-fluoren-2-yl)-1-phenyl-1H-benzo[d]imdiazolato)(acetylacetonate)iridium(III) (Ir(fbi)₂(acac)), bis(2-phenylpyridine)(3-(pyridine-2-yl)-2H-chromen-2-onate)iridium(III) (fac-Ir(ppy)₂Pc), bis(2-(2,4-difluorophenyl)quinoline)(picolinate)iridium(III) (FPQIrpic), bis(4-phenylthieno[3,2-c]pyridinato-N,C2′) (acetylacetonate) iridium(III) (PO-01) and combinations thereof.

The contents of the yellow-green host in the third layer 740C can be about 50 wt % to about 99 wt %, for example, about 80 wt % to about 95 wt %, and the contents of the yellow-green emitter in the third layer 740C can be about 1 wt % to about 50 wt %, for example, about 5 wt % to about 20 wt %, but is not limited thereto. When the third layer 740C comprises both the P-type yellow-green host and the N-type yellow-green host, the P-type yellow-green host and the N-type yellow-green host can be admixed, but is not limited to, with a weight ratio of about 4:1 to about 1:4, for example about 3:1 to about 1:3.

The OLED D3 with a tandem structure in accordance with this embodiment comprises at least one of the organic compounds having the structure of Chemical Formulae 1 to 3 in the at least one emitting material layer. Since the organic compounds may have a wide plate-like structure, the organic compounds can receive efficiently singlet exciton energy from the first host 744 and/or the second host 746. The OLED D3 with three emitting parts including the organic compound can implement white emission with improved luminous efficiency and the luminous lifespan. In addition, the organic light emitting diode can comprise four or more emitting parts.

### Synthesis Example 1: Synthesis of Compound 1-1

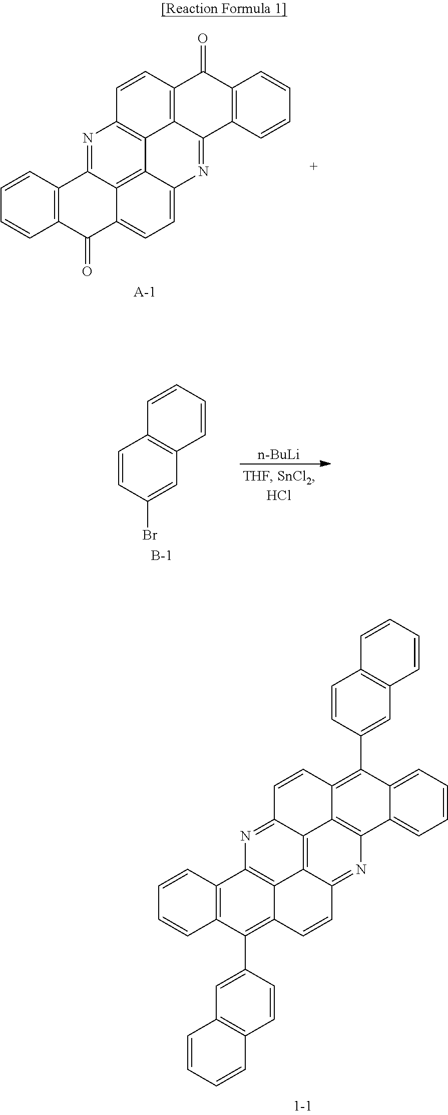

Compound A-1 (3.0 g, 7.3 mmol) and compound B-1 (4.55 g, 22.1 mmol) dissolved in anhydrous tetrahydrofuran (THF, 50 ml) were added into a 100 ml round bottom flask under nitrogen atmosphere, and then the solution was stirred at −78° C. n-BuLi (8.8 ml, 2.5 M) were added dropwisely into the round bottom flask and then the solution was stirred again for 3 hours. After the reaction was complete, the temperature was raised to a room temperature, and then the reactants were stirred for 12 hours. 3M HCl solution (50 ml) and SnCl₂ (2.08 g, 11 mmol) were added into the round bottom flask under nitrogen atmosphere, and the solution was stirred for 3 hours. Triethylamine was added into the solution to adjust pH of the solution to be neutral, the solution was stirred for 3 hours. Organic phase was extracted with water and dichloromethane and treated with anhydrous MgSO₄. The organic phase was filtered and subjected to reduced pressure to obtain a crude product. The crude product was purified with a column chromatography (eluent: dichloromethane) and recrystallized to give a solid Compound 1-1 (0.55 g, 12%).

### Synthesis Example 2: Synthesis of Compound 1-2

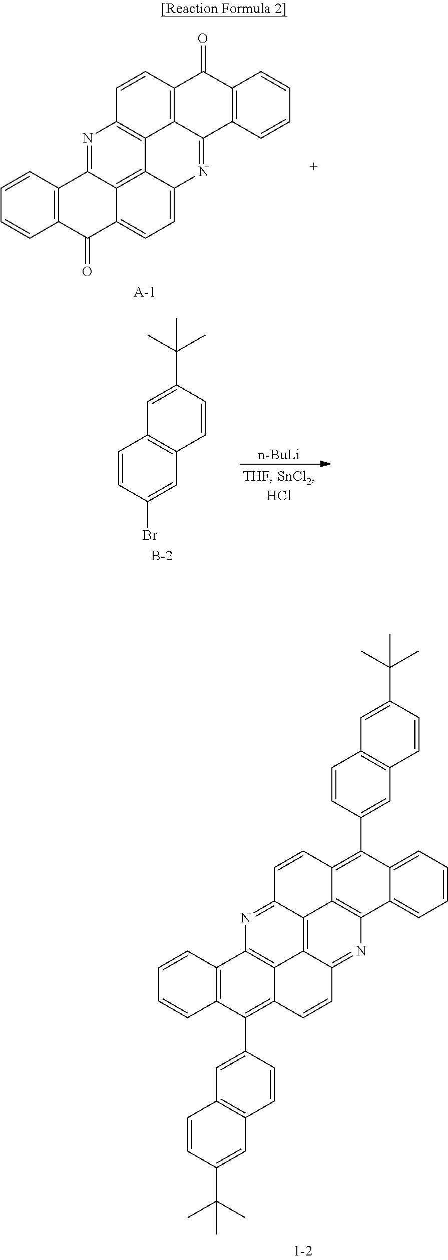

Compound A-1 (3.0 g, 7.3 mmol) and compound B-2 (5.79 g, 22.1 mmol) dissolved in anhydrous tetrahydrofuran (THF, 50 ml) were added into a 100 ml round bottom flask under nitrogen atmosphere, and then the solution was stirred at −78° C. n-BuLi (8.8 ml, 2.5 M) were added dropwisely into the round bottom flask and then the solution was stirred again for 3 hours. After the reaction was complete, the temperature was raised to a room temperature, and then the reactants were stirred for 12 hours. 3M HCl solution (50 ml) and SnCl₂ (2.08 g, 11 mmol) were added into the round bottom flask under nitrogen atmosphere, and the solution was stirred for 3 hours. Triethylamine was added into the solution to adjust pH of the solution to be neutral, the solution was stirred for 3 hours. Organic phase was extracted with water and dichloromethane and treated with anhydrous MgSO₄. The organic phase was filtered and subjected to under pressure to obtain a crude product. The crude product was purified with a column chromatography (eluent: dichloromethane) and recrystallized to give a solid Compound 1-2 (0.71 g, 13%).

### Synthesis Example 3: Synthesis of Compound 1-3

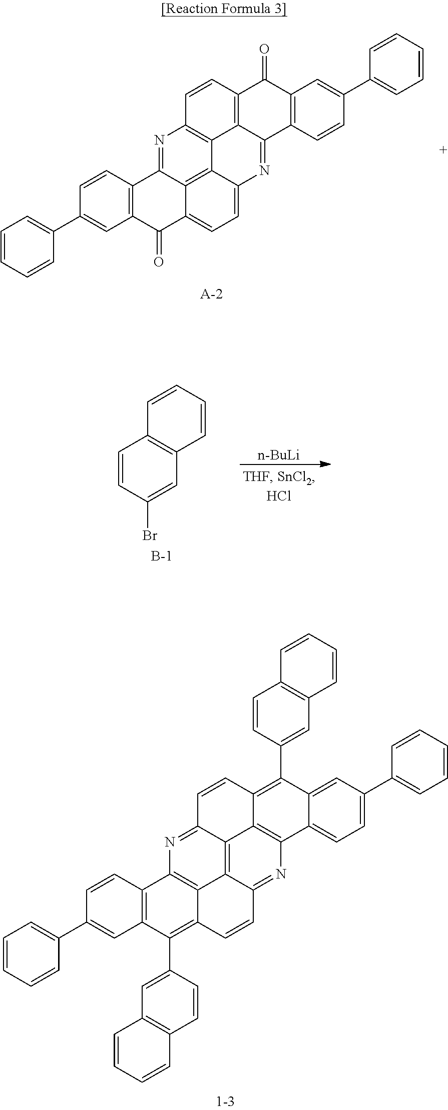

Compound A-2 (4.1 g, 7.3 mmol) and compound B-1 (4.55 g, 22.1 mmol) dissolved in anhydrous tetrahydrofuran (THF, 50 ml) were added into a 100 ml round bottom flask under nitrogen atmosphere, and then the solution was stirred at −78° C. n-BuLi (8.8 ml, 2.5 M) were added dropwisely into the round bottom flask and then the solution was stirred again for 3 hours. After the reaction was complete, the temperature was raised to a room temperature, and then the reactants were stirred for 12 hours. 3M HCl solution (50 ml) and SnCl₂ (2.08 g, 11 mmol) were added into the round bottom flask under nitrogen atmosphere, and the solution was stirred for 3 hours. Triethylamine was added into the solution to adjust pH of the solution to be neutral, the solution was stirred for 3 hours. Organic phase was extracted with water and dichloromethane and treated with anhydrous MgSO₄. The organic phase was filtered and subjected to reduced pressure to obtain a crude product. The crude product was purified with a column chromatography (eluent: dichloromethane) and recrystallized to give a solid Compound 1-3 (0.42 g, 10%).

### Synthesis Example 4: Synthesis of Compound 1-4

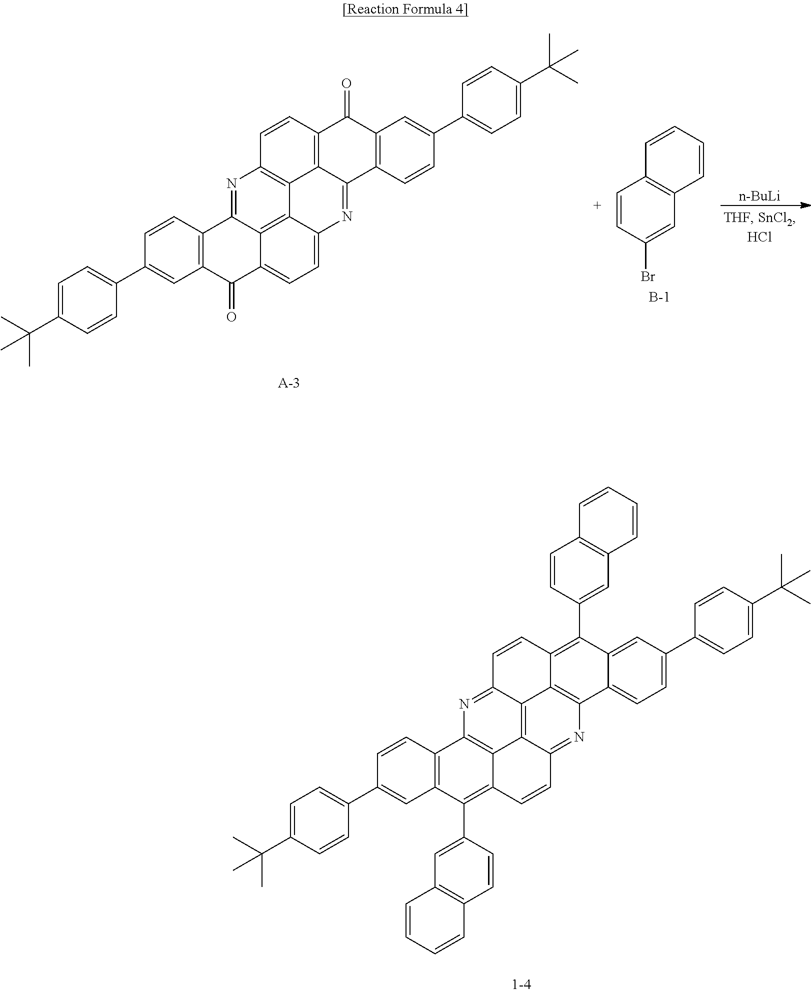

Compound A-3 (4.93 g, 7.3 mmol) and compound B-1 (4.55 g, 22.1 mmol) dissolved in anhydrous tetrahydrofuran (THF, 50 ml) were added into a 100 ml round bottom flask under nitrogen atmosphere, and then the solution was stirred at −78° C. n-BuLi (8.8 ml, 2.5 M) were added dropwisely into the round bottom flask and then the solution was stirred again for 3 hours. After the reaction was complete, the temperature was raised to a room temperature, and then the reactants were stirred for 12 hours. 3M HCl solution (50 ml) and SnCl₂ (2.08 g, 11 mmol) were added into the round bottom flask under nitrogen atmosphere, and the solution was stirred for 3 hours. Triethylamine was added into the solution to adjust pH of the solution to be neutral, the solution was stirred for 3 hours. Organic phase was extracted with water and dichloromethane and treated with anhydrous MgSO₄. The organic phase was filtered and subjected to reduced pressure to obtain a crude product. The crude product was purified with a column chromatography (eluent: dichloromethane) and recrystallized to give a solid Compound 1-4 (0.44 g, 11%).

### Synthesis Example 5: Synthesis of Compound 1-5

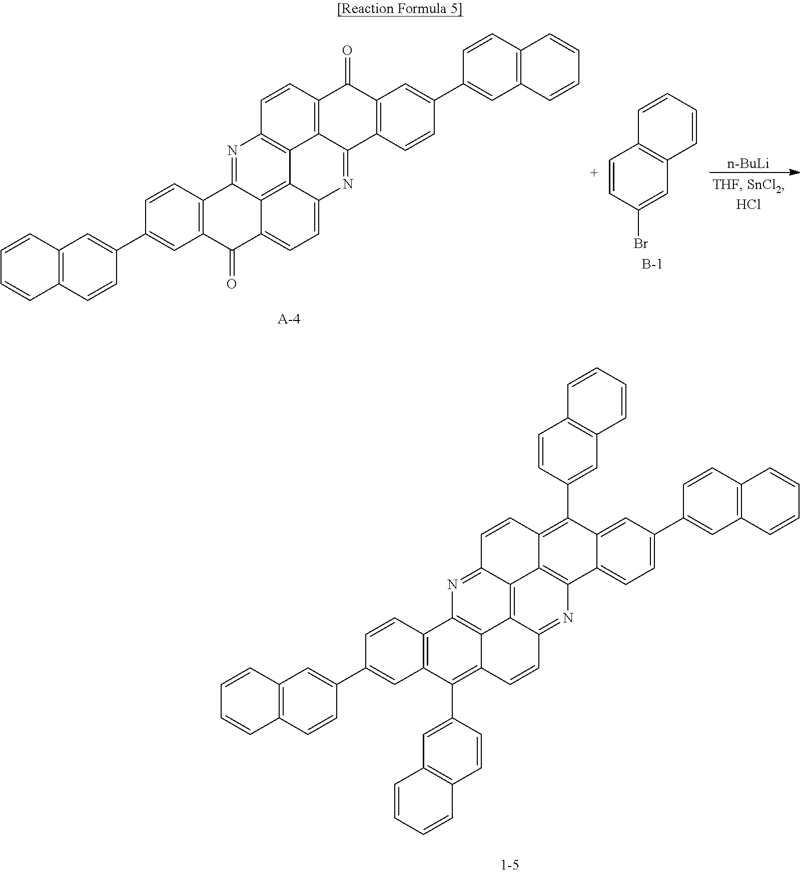

Compound A-4 (4.9 g, 7.3 mmol) and compound B-1 (4.55 g, 22.1 mmol) dissolved in anhydrous tetrahydrofuran (THF, 50 ml) were added into a 100 ml round bottom flask under nitrogen atmosphere, and then the solution was stirred at −78° C. n-BuLi (8.8 ml, 2.5 M) were added dropwisely into the round bottom flask and then the solution was stirred again for 3 hours. After the reaction was complete, the temperature was raised to a room temperature, and then the reactants were stirred for 12 hours. 3M HCl solution (50 ml) and SnCl₂ (2.08 g, 11 mmol) were added into the round bottom flask under nitrogen atmosphere, and the solution was stirred for 3 hours. Triethylamine was added into the solution to adjust pH of the solution to be neutral, the solution was stirred for 3 hours. Organic phase was extracted with water and dichloromethane and treated with anhydrous MgSO₄. The organic phase was filtered and subjected to reduced pressure to obtain a crude product. The crude product was purified with a column chromatography (eluent: dichloromethane) and recrystallized to give a solid Compound 1-5 (0.16 g, 11%).

### Synthesis Example 6: Synthesis of Compound 1-6

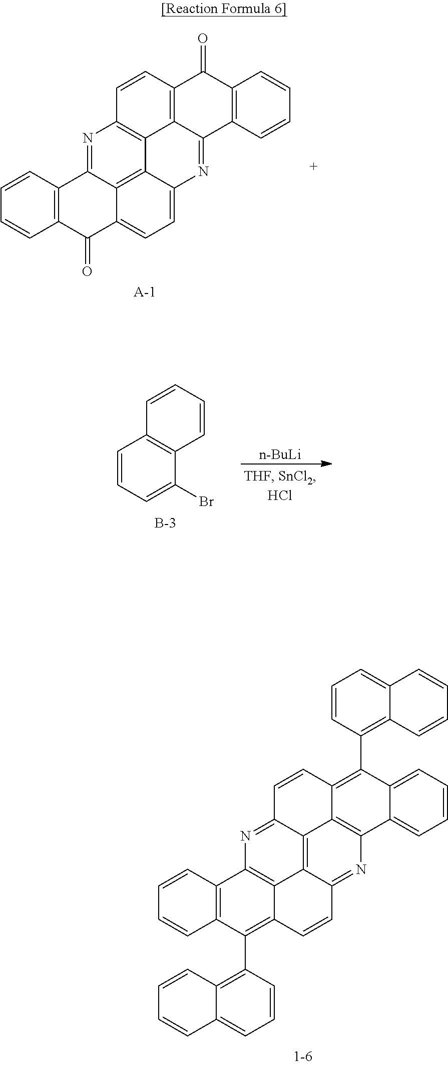

Compound A-1 (3.0 g, 7.3 mmol) and compound B-3 (4.55 g, 22.1 mmol) dissolved in anhydrous tetrahydrofuran (THF, 50 ml) were added into a 100 ml round bottom flask under nitrogen atmosphere, and then the solution was stirred at −78° C. n-BuLi (8.8 ml, 2.5 M) were added dropwisely into the round bottom flask and then the solution was stirred again for 3 hours. After the reaction was complete, the temperature was raised to a room temperature, and then the reactants were stirred for 12 hours. 3M HCl solution (50 ml) and SnCl₂ (2.08 g, 11 mmol) were added into the round bottom flask under nitrogen atmosphere, and the solution was stirred for 3 hours. Triethylamine was added into the solution to adjust pH of the solution to be neutral, the solution was stirred for 3 hours. Organic phase was extracted with water and dichloromethane and treated with anhydrous MgSO₄. The organic phase was filtered and subjected to reduced pressure to obtain a crude product. The crude product was purified with a column chromatography (eluent: dichloromethane) and recrystallized to give a solid Compound 1-6 (0.69 g, 15%).

### Synthesis Example 7: Synthesis of Compound 1-7

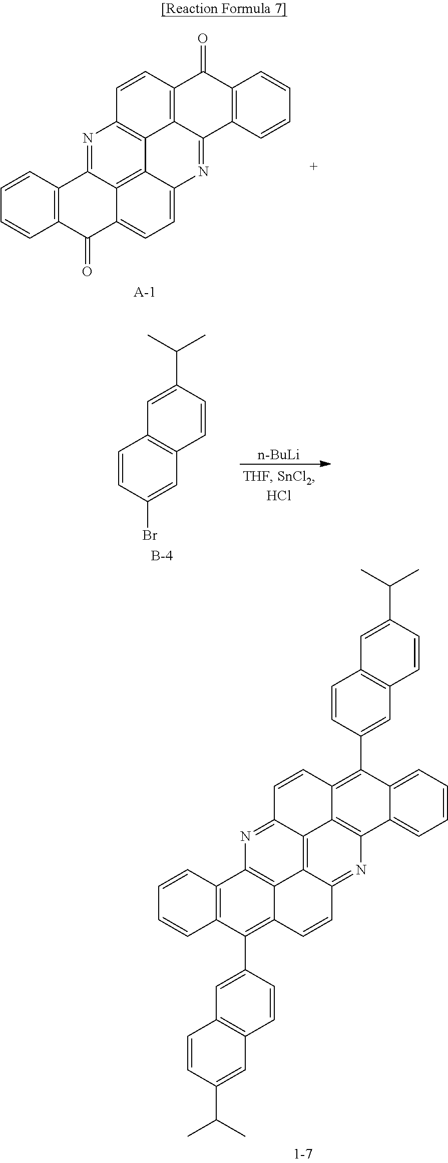

Compound A-1 (3.01 g, 7.3 mmol) and compound B-4 (5.48 g, 22.1 mmol) dissolved in anhydrous tetrahydrofuran (THF, 50 ml) were added into a 100 ml round bottom flask under nitrogen atmosphere, and then the solution was stirred at −78° C. n-BuLi (8.8 ml, 2.5 M) were added dropwisely into the round bottom flask and then the solution was stirred again for 3 hours. After the reaction was complete, the temperature was raised to a room temperature, and then the reactants were stirred for 12 hours. 3M HCl solution (50 ml) and SnCl₂ (2.08 g, 11 mmol) were added into the round bottom flask under nitrogen atmosphere, and the solution was stirred for 3 hours. Triethylamine was added into the solution to adjust pH of the solution to be neutral, the solution was stirred for 3 hours. Organic phase was extracted with water and dichloromethane and treated with anhydrous MgSO₄. The organic phase was filtered and subjected to reduced pressure to obtain a crude product. The crude product was purified with a column chromatography (eluent: dichloromethane) and recrystallized to give a solid Compound 1-7 (0.53 g, 10%).

### Synthesis Example 8: Synthesis of Compound 1-17

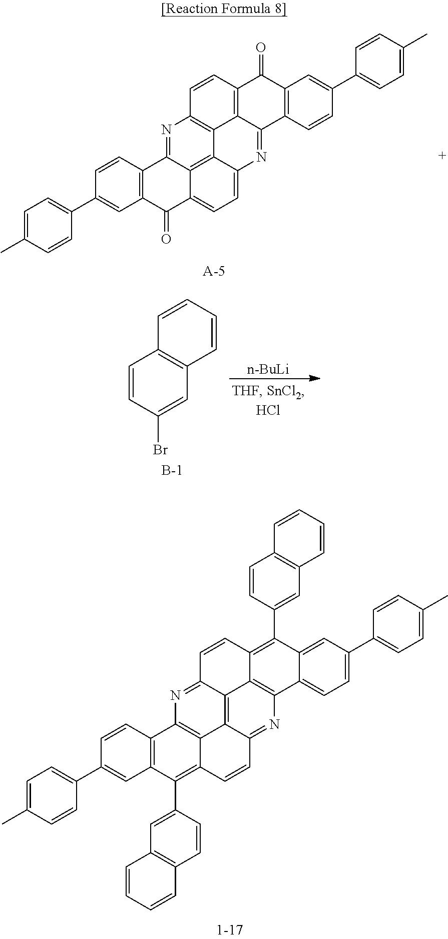

Compound A-5 (4.3 g, 7.3 mmol) and compound B-1 (4.55 g, 22.1 mmol) dissolved in anhydrous tetrahydrofuran (THF, 50 ml) were added into a 100 ml round bottom flask under nitrogen atmosphere, and then the solution was stirred at −78° C. n-BuLi (8.8 ml, 2.5 M) were added dropwisely into the round bottom flask and then the solution was stirred again for 3 hours. After the reaction was complete, the temperature was raised to a room temperature, and then the reactants were stirred for 12 hours. 3M HCl solution (50 ml) and SnCl₂ (2.08 g, 11 mmol) were added into the round bottom flask under nitrogen atmosphere, and the solution was stirred for 3 hours. Triethylamine was added into the solution to adjust pH of the solution to be neutral, the solution was stirred for 3 hours. Organic phase was extracted with water and dichloromethane and treated with anhydrous MgSO₄. The organic phase was filtered and subjected to reduced pressure to obtain a crude product. The crude product was purified with a column chromatography (eluent: dichloromethane) and recrystallized to give a solid Compound 1-17 (0.45 g, 12%).

### Example 1 (Ex. 1): Fabrication of OLED

An organic light emitting diode where Compound 1-1 of Synthesis Example 1 as an emitter were applied into an emitting material layer was fabricated. A glass substrate onto which ITO (50 nm) was coated as a thin film was washed and ultrasonically cleaned by solvent such as isopropyl alcohol, acetone and dried at 100° C. oven. The substrate was transferred to a vacuum chamber for depositing emissive layer. Subsequently, an emissive layer and a cathode were deposited by evaporation from a heating boat under about 5-7×10⁻⁷ Torr with setting a deposition rate 1 Å/s as the following order:

A hole injection layer (HIL including HAT-CN, 7 nm); a hole transport layer (HTL including NPB, 78 nm); an electron blocking layer (EBL including TAPC, 10 nm); an emitting material layer (EML including Compound 2-1 in Chemical Formula 6 (mCBP, 64 wt %), NH below (35 wt %), Compound 1-1 (1 wt %), 38 nm); a hole blocking layer (HBL including B3PYMPM, 10 nm); an electron transport layer (ETL including TPBi, 25 nm); an electron injection layer (LiF, 1 nm): and a cathode (Al, 100 nm).

The structures of a hole injection material (HAT-CN), a hole transporting material (NPB), an electron blocking material (TAPC), a hole blocking material (B3PYMPM) and an electron transporting material (ETL) are illustrated as follows:

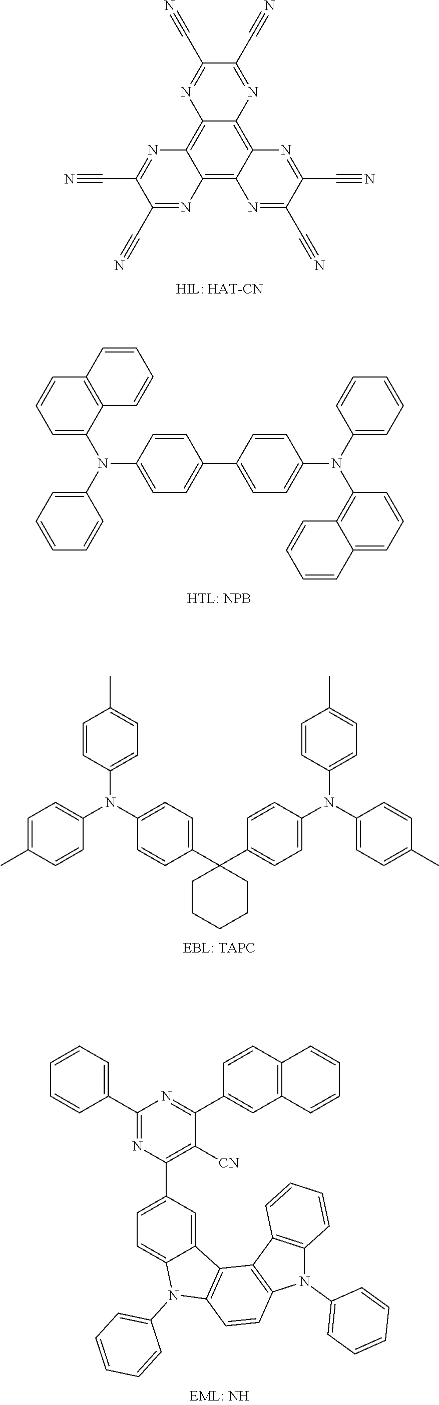

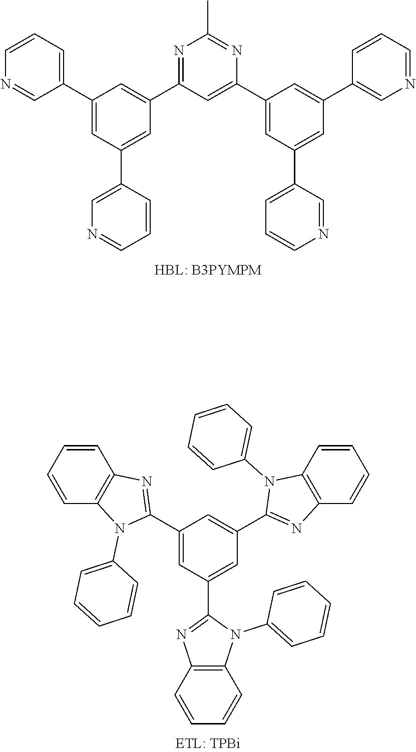 [SMILES: Cc1nc(-c2cc(-c3cccnc3)cc(-c3cccnc3)c2)cc(-c2cc(-c3cccnc3)cc(-c3cccnc3)c2)n1 ; c1ccc(-n2c(-c3cc(-c4nc5ccccc5n4-c4ccccc4)cc(-c4nc5ccccc5n4-c4ccccc4)c3)nc3ccccc32)cc1]

### Examples 2-6 (Ex. 2-6): Fabrication of OLEDs

An OLED was fabricated using the same procedure and the same materials as Example 1, except that each of Compound 1-2 (Ex. 2), Compound 1-3 (Ex. 3), Compound 1-4 (Ex. 4), Compound 1-5 (Ex. 5) and Compound 1-6 (Ex. 6) instead of Compound 1-1 was used as the emitter in the emitting material layer, respectively.

### Comparative Examples 1-8 (Refs. 1-8): Fabrication of OLED

An OLED was fabricated using the same procedure and the same materials as Example 1, except that each of the following Compound Ref. 1-1 (Ref 1), Compound Ref. 1-2 (Ref 2), Compound Ref. 1-3 (Ref. 3), Compound Ref. 1-4 (Ref. 4), Compound Ref. 1-5 (Ref. 5), Compound Ref. 1-6 (Ref. 6), Compound Ref. 1-7 (Ref 7) and Compound Ref. 1-8 (Ref 8) instead of Compound 1-1 was used as the emitter in the emitting material layer, respectively.

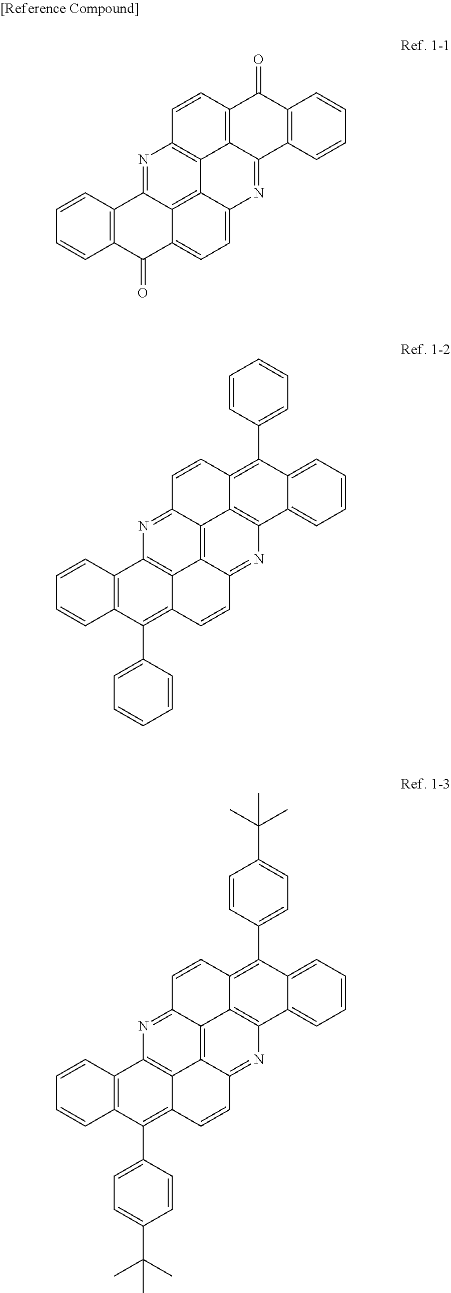 [SMILES: O=c1c2ccccc2c2nc3ccc4c(=O)c5ccccc5c5nc6ccc1c2c6c3c45 ; c1ccc(-c2c3ccccc3c3nc4ccc5c(-c6ccccc6)c6ccccc6c6nc7ccc2c3c7c4c56)cc1 ; CC(C)(C)c1ccc(-c2c3ccccc3c3nc4ccc5c(-c6ccc(C(C)(C)C)cc6)c6ccccc6c6nc7ccc2c3c7c4c56)cc1]

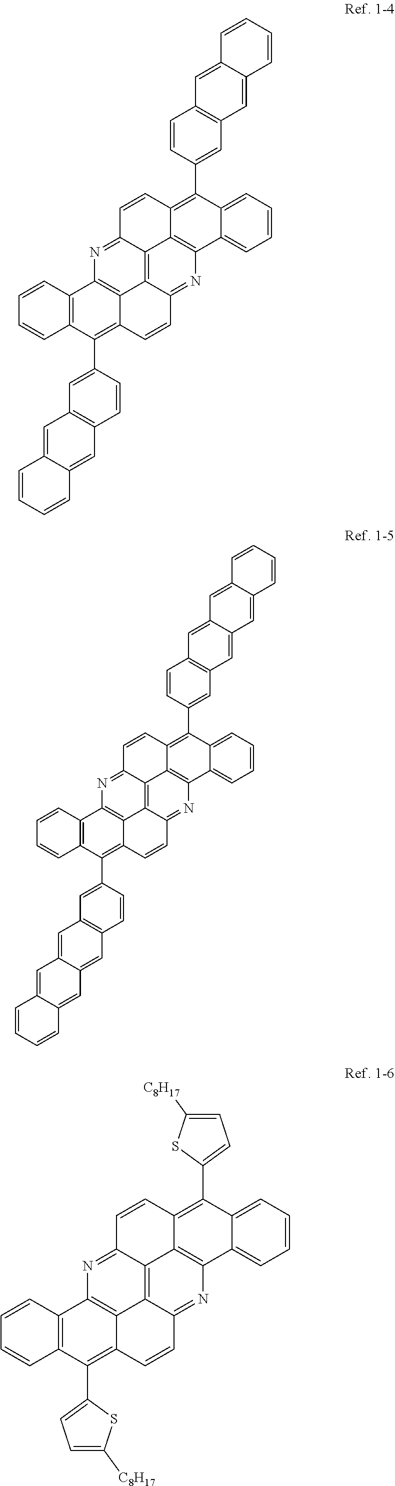

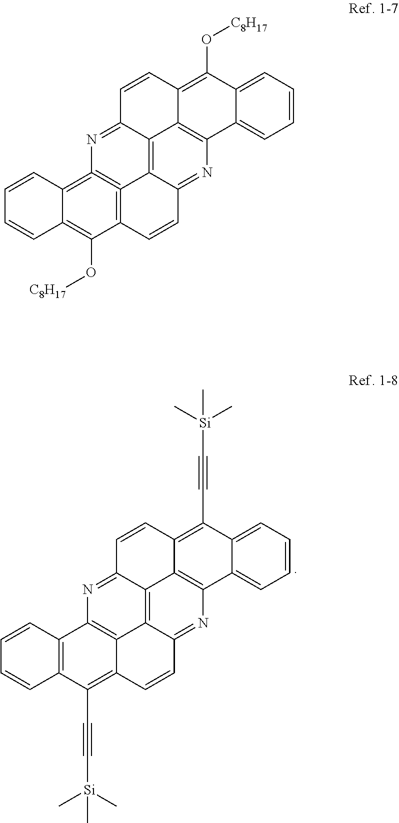

### Experimental Example 1: Measurement of Energy Level and Dipole Moment of Compounds

Luminous property for the OLEDs fabricated in Examples 1 to 6 and Comparative Examples 1 to 8 was measured. Each of the OLEDs having luminous area of 9 mm² was connected to external power source and the luminous property was measured using a current source (KEITHLEY) and a photometer (PR650) at a room temperature. In particular, driving voltage (V), external quantum efficiency (EQE, relative value) and lifespan (LT₉₅, relative value) at which the luminance was reduced to 95% from initial luminance was measured at a current density 6 mA/cm². The measurement results are indicated in the following Table 1.

TABLE 1
Luminous Properties of OLED

| Sample | Emitter | V | EQE (%) | LT₉₅ (%) |
|---|---|---|---|---|
| Ref. 1 | Ref. 1-1 | 3.7 | 96% | 38% |
| Ref. 2 | Ref. 1-2 | 3.7 | 100% | 100% |
| Ref. 3 | Ref. 1-3 | 3.7 | 98% | 100% |
| Ref. 4 | Ref. 1-4 | 3.7 | 99% | 92% |
| Ref. 5 | Ref. 1-5 | 3.7 | 97% | 88% |
| Ref. 6 | Ref. 1-6 | 3.6 | 85% | 40% |
| Ref. 7 | Ref. 1-7 | 3.6 | 88% | 37% |
| Ref. 8 | Ref. 1-8 | 3.5 | 67% | 31% |
| Ex. 1 | 1-1 | 3.7 | 107% | 127% |
| Ex. 2 | 1-2 | 3.7 | 110% | 134% |
| Ex. 3 | 1-3 | 3.7 | 106% | 129% |
| Ex. 4 | 1-4 | 3.7 | 108% | 133% |
| Ex. 5 | 1-5 | 3.7 | 107% | 131% |
| Ex. 6 | 1-6 | 3.7 | 104% | 124% |

As indicated in Table 1, compared to the OLED fabricated in Ref. 2 where Compound Ref. 1-2 having a phenyl group substituted to the core was used as the emitter, in the OLED fabricated in Ref 1 where Ref. 1-1 having an oxygen atom linked to the core by exocyclic bond was used as the emitter, and in Ref 4 to Ref. 8 where Compounds Ref. 1-4 to Ref. 1-8, an anthracenyl group with three fused aromatic rings, a tetracene group with four fused aromatic rings, a hetero aryl group, an alkoxy group or a silyl group is substituted to the core, were used as the emitter, EQE and the luminous lifespan were reduced. On the other hand, compared to the OLED fabricated in Ref 2, in the OLED fabricated in Ex. 1 to Ex. 6 where compounds having a naphthyl group is substituted to the core, driving voltage was equivalent level, but EQE and luminous lifespan was improved significantly.

It will be apparent to those skilled in the art that various modifications and variations may be made in the present disclosure without departing from the scope of the disclosure. Thus, it is intended that the present disclosure cover the modifications and variations of the present disclosure provided they come within the scope of the appended claims.

What is claimed is:

1. An organic compound of Chemical Formula 1: 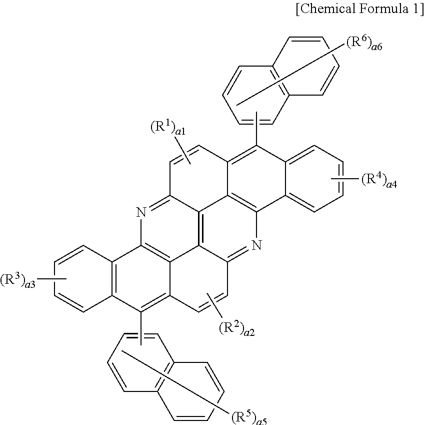
wherein, in Chemical Formula 1,
each of R¹, R², R³, R⁴, R⁵ and R⁶ is independently a halogen atom, a cyano group, an unsubstituted or substituted C₁-C₂₀ alkyl group, an unsubstituted or substituted C₁-C₂₀ alkyl amino group, an unsubstituted or substituted C₆-C₃₀ aryl group, an unsubstituted or substituted C₃-C₃₀ hetero aryl group, an unsubstituted or substituted C₆-C₃₀ aryl amino group or an unsubstituted or substituted C₃-C₃₀ hetero aryl amino group, where each R¹ is identical to or different from each other when a1 is 2, each R² is identical to or different from each other when a2 is 2, each R³ is identical to or different from each other when a3 is 2, 3 or 4, each R⁴ is identical to or different from each other when a4 is 2, 3 or 4, each R⁵ is identical to or different from each other when a5 is 2, 3, 4, 5, 6 or 7 and each R⁶ is identical to or different from each other when a6 is 2, 3, 4, 5, 6 or 7;
each of a1 and a2 is independently an integer of 0, 1 or 2;
each of a3 and a4 is independently an integer of 0, 1, 2, 3 or 4; and
each of a5 and a6 is independently an integer of 0, 1, 2, 3, 4, 5, 6 or 7.

2. The organic compound of claim 1, wherein the organic compound has a structure of Chemical Formula 2A or Chemical Formula 2B: 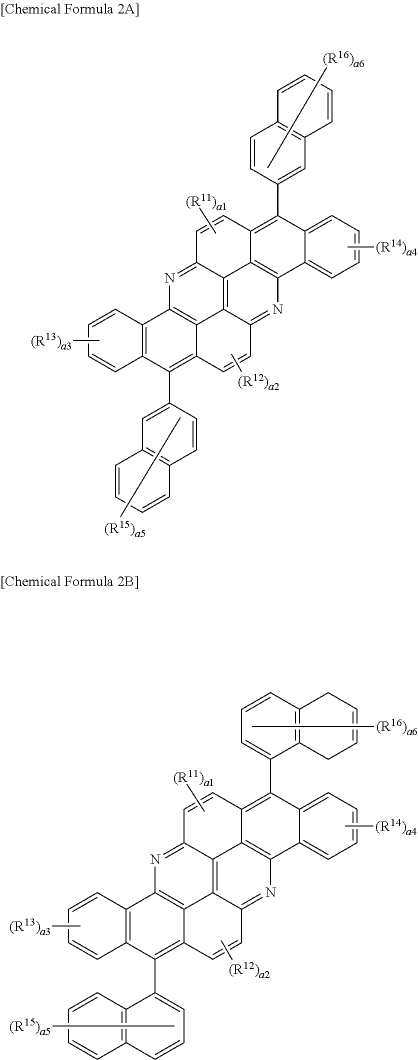
wherein, in Chemical Formulae 2A and 2B,
each of a1, a2, a3, a4, a5 and a6 is the same as defined in Chemical Formula 1,
each of R¹¹, R¹², R¹³, R¹⁴, R¹⁵ and R¹⁶ is independently an unsubstituted or substituted C₁-C₂₀ alkyl group or an unsubstituted or C₁-C₁₀ alkyl-substituted C₆-C₃₀ aryl group, where each R¹¹ is identical to or different from each other when a1 is 2, each R¹² is identical to or different from each other when a2 is 2, each R¹³ is identical to or different from each other when a3 is 2, 3 or 4, each R¹⁴ is identical to or different from each other when a4 is 2, 3 or 4, each R¹⁵ is identical to or different from each other when a5 is 2, 3, 4, 5, 6 or 7 and each R¹⁶ is identical to or different from each other when a6 is 2, 3, 4, 5, 6 or 7.

3. The organic compound of claim 1, wherein each of R³, R⁴, R⁵ and R⁶ is independently an unsubstituted or substituted C₁-C₁₀ alkyl group or an unsubstituted or C₁-C₁₀ alkyl-substituted C₆-C₃₀ aryl group, each of a1 and a2 is 0, and each of a3, a4, a5 and a6 is independently 0 or 1.

4. The organic compound of claim 1, wherein each of a1 and a2 is 0, each of R³ and R⁴ is independently an unsubstituted or C₁-C₁₀ alkyl-substituted C₆-C₃₀ aryl group, each of a3 and a4 is independently 0 or 1, each of R⁵ and R⁶ is independently an unsubstituted or substituted C₁-C₁₀ alkyl group, and each of a5 and a6 is independently 0 or 1.

5. The organic compound of claim 1, wherein each of a1 to a4 is 0, each of R⁵ and R⁶ is independently an unsubstituted or substituted C₁-C₁₀ alkyl group, and each of a5 and a6 is independently 0 or 1.

6. The organic compound of claim 2, wherein the organic compound has the structure of Chemical Formula 2A, each of a1 to a4 is 0, each of R¹⁵ and R¹⁶ is independently an unsubstituted or substituted C₁-C₁₀ alkyl group, and each of a5 and a6 is independently 0 or 1.

7. The organic compound of claim 1, wherein the organic compound is at least one of the following compounds 1-1 to 1-56:

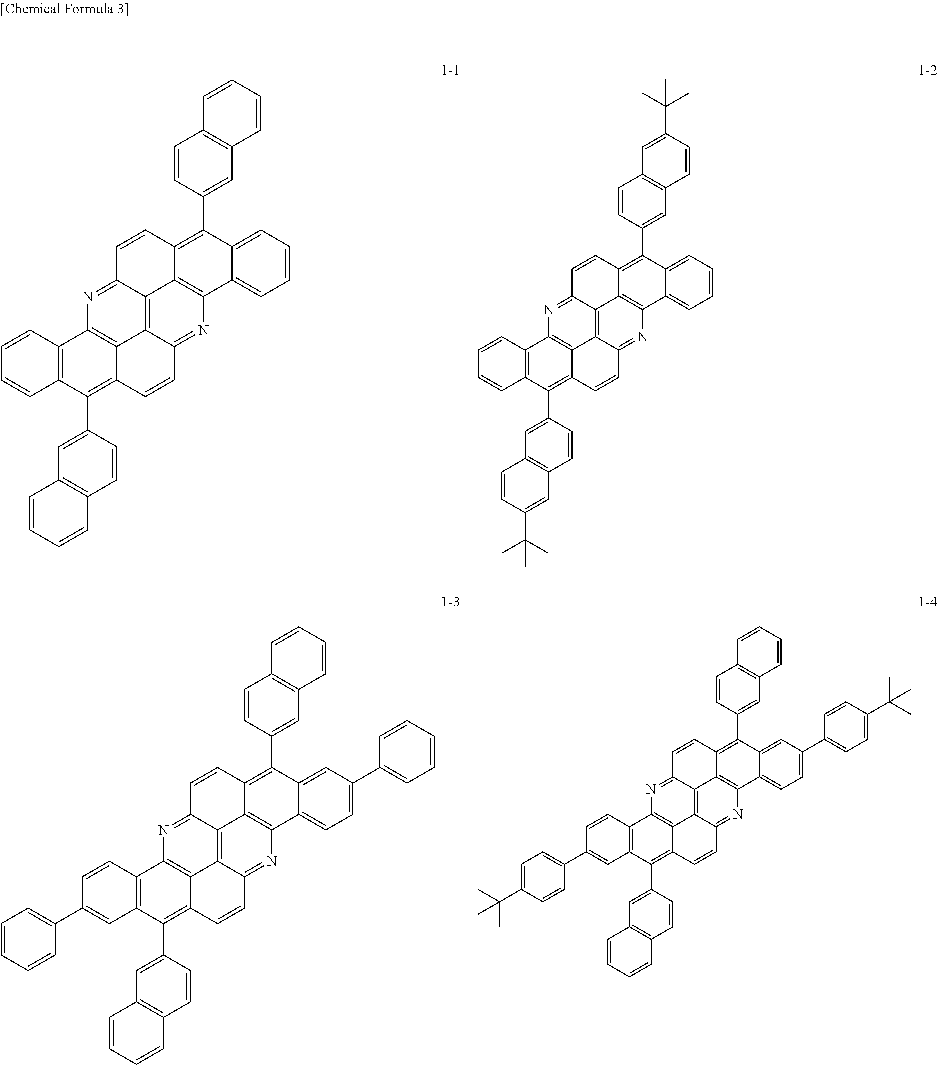 [SMILES: c1ccc2cc(-c3c4ccccc4c4nc5ccc6c(-c7ccc8ccccc8c7)c7ccccc7c7nc8ccc3c4c8c5c67)ccc2c1 ; CC(C)(C)c1ccc2cc(-c3c4ccccc4c4nc5ccc6c(-c7ccc8cc(C(C)(C)C)ccc8c7)c7ccccc7c7nc8ccc3c4c8c5c67)ccc2c1 ; c1ccc(-c2ccc3c(c2)c(-c2ccc4ccccc4c2)c2ccc4nc5c6ccc(-c7ccccc7)cc6c(-c6ccc7ccccc7c6)c6ccc7nc3c2c4c7c65)cc1 ; CC(C)(C)c1ccc(-c2ccc3c(c2)c(-c2ccc4ccccc4c2)c2ccc4nc5c6ccc(-c7ccc(C(C)(C)C)cc7)cc6c(-c6ccc7ccccc7c6)c6ccc7nc3c2c4c7c65)cc1]

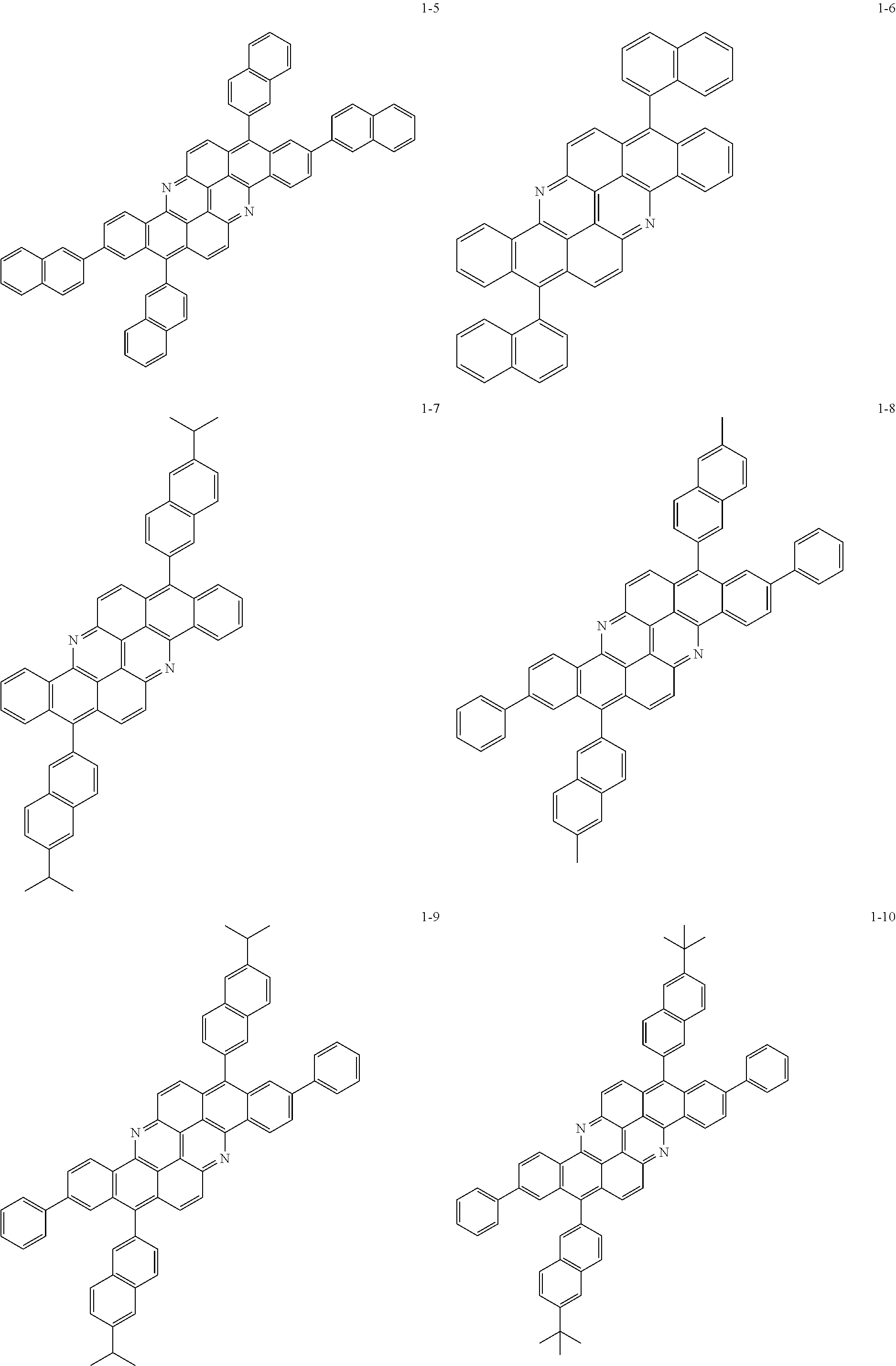

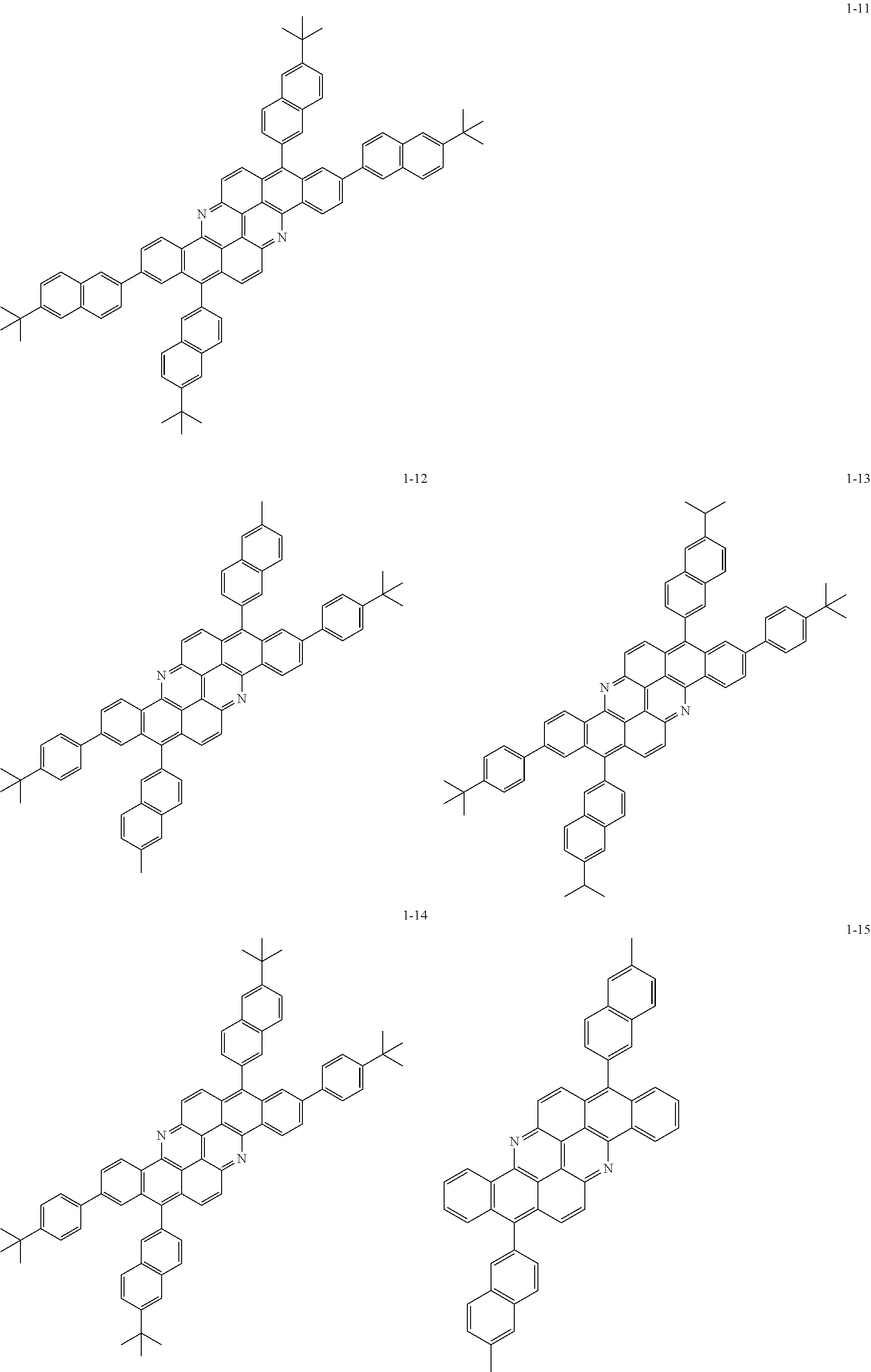

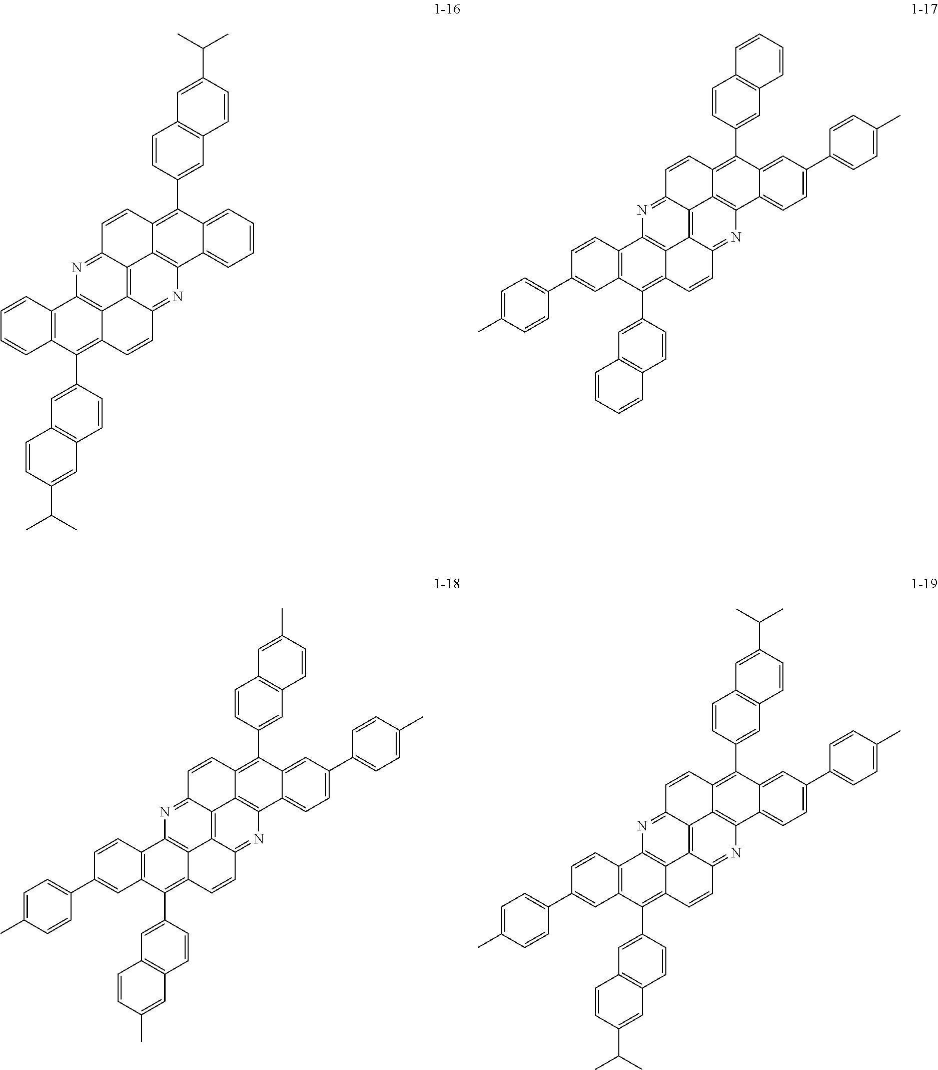

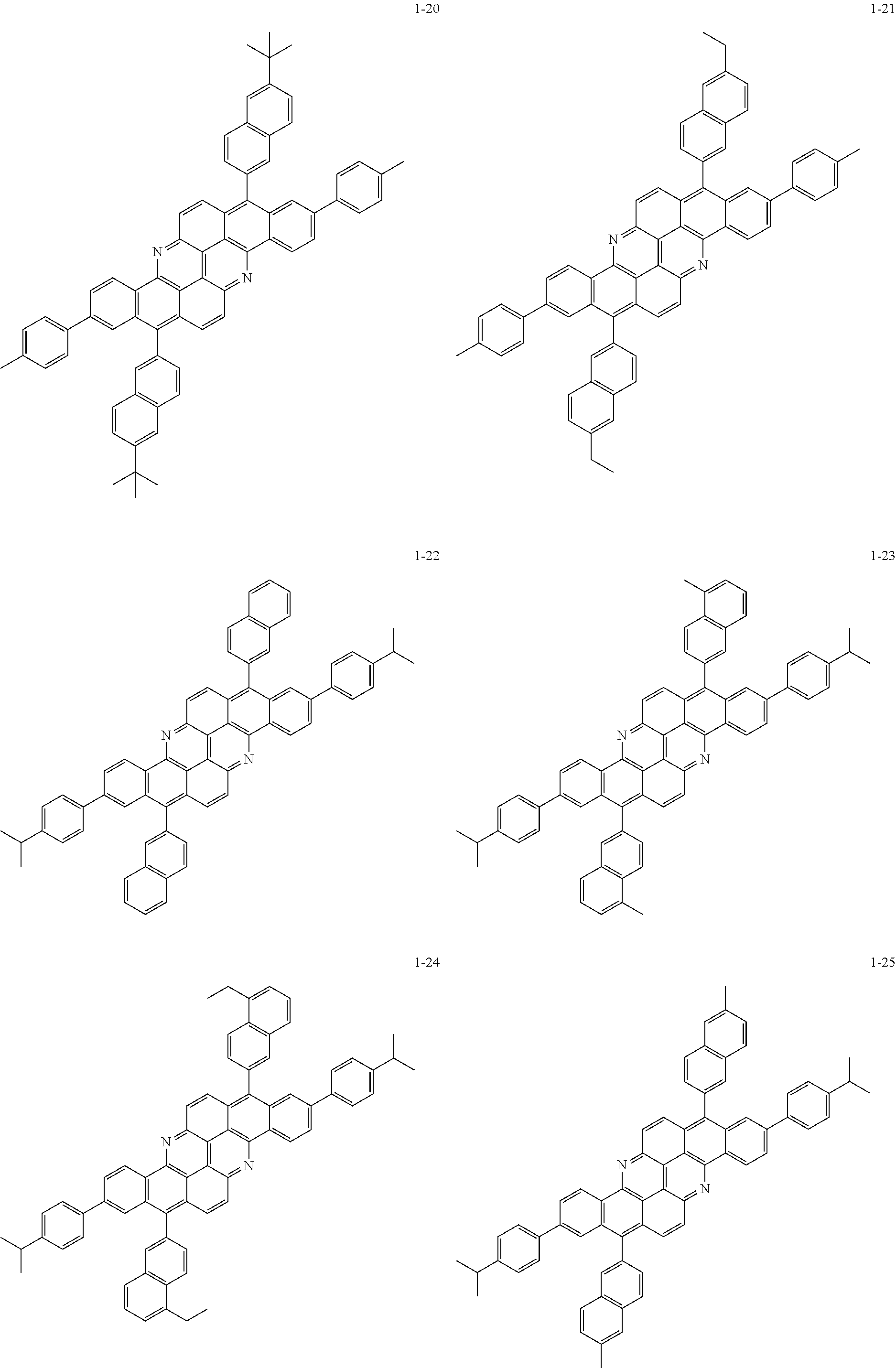

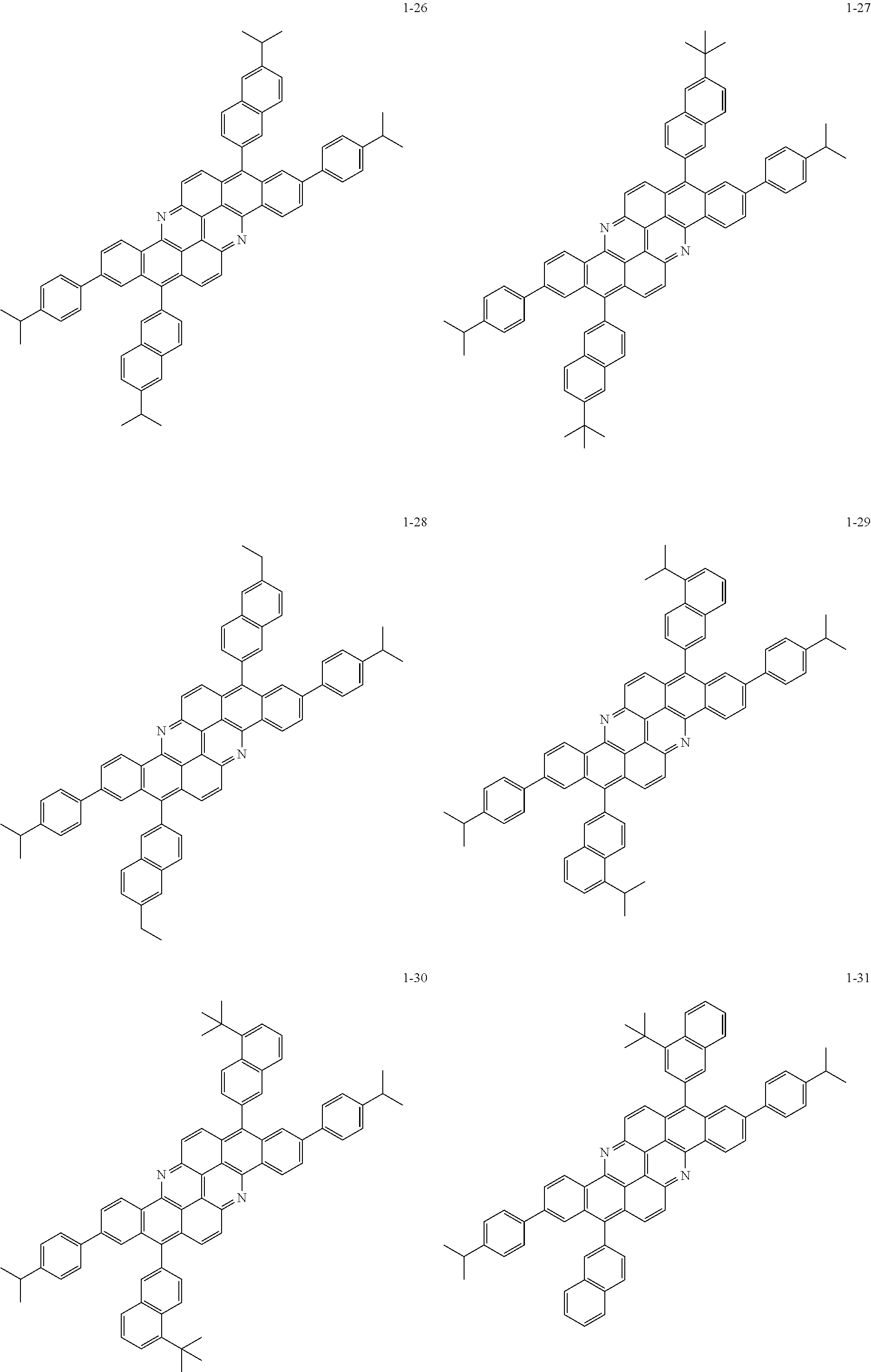

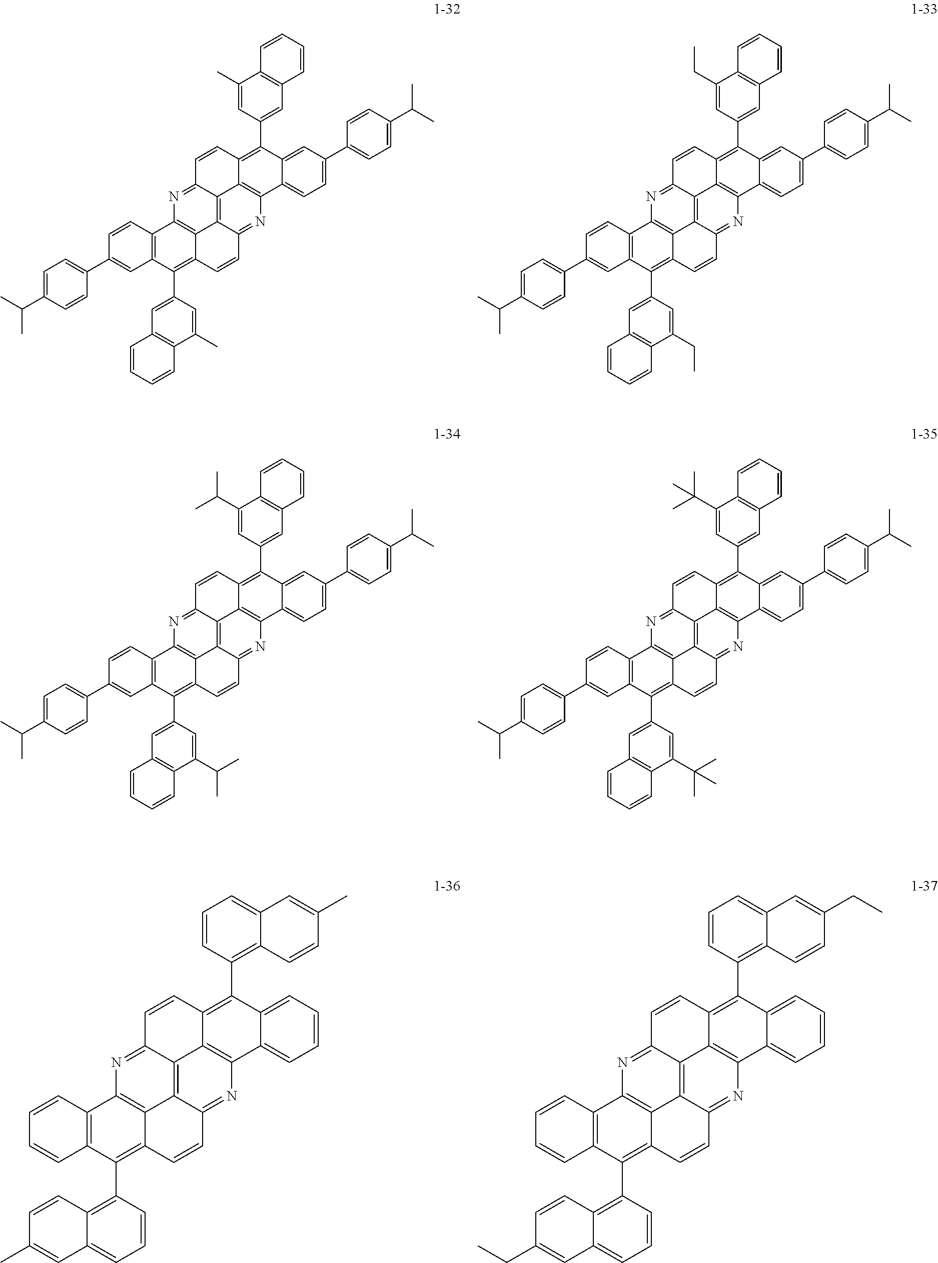

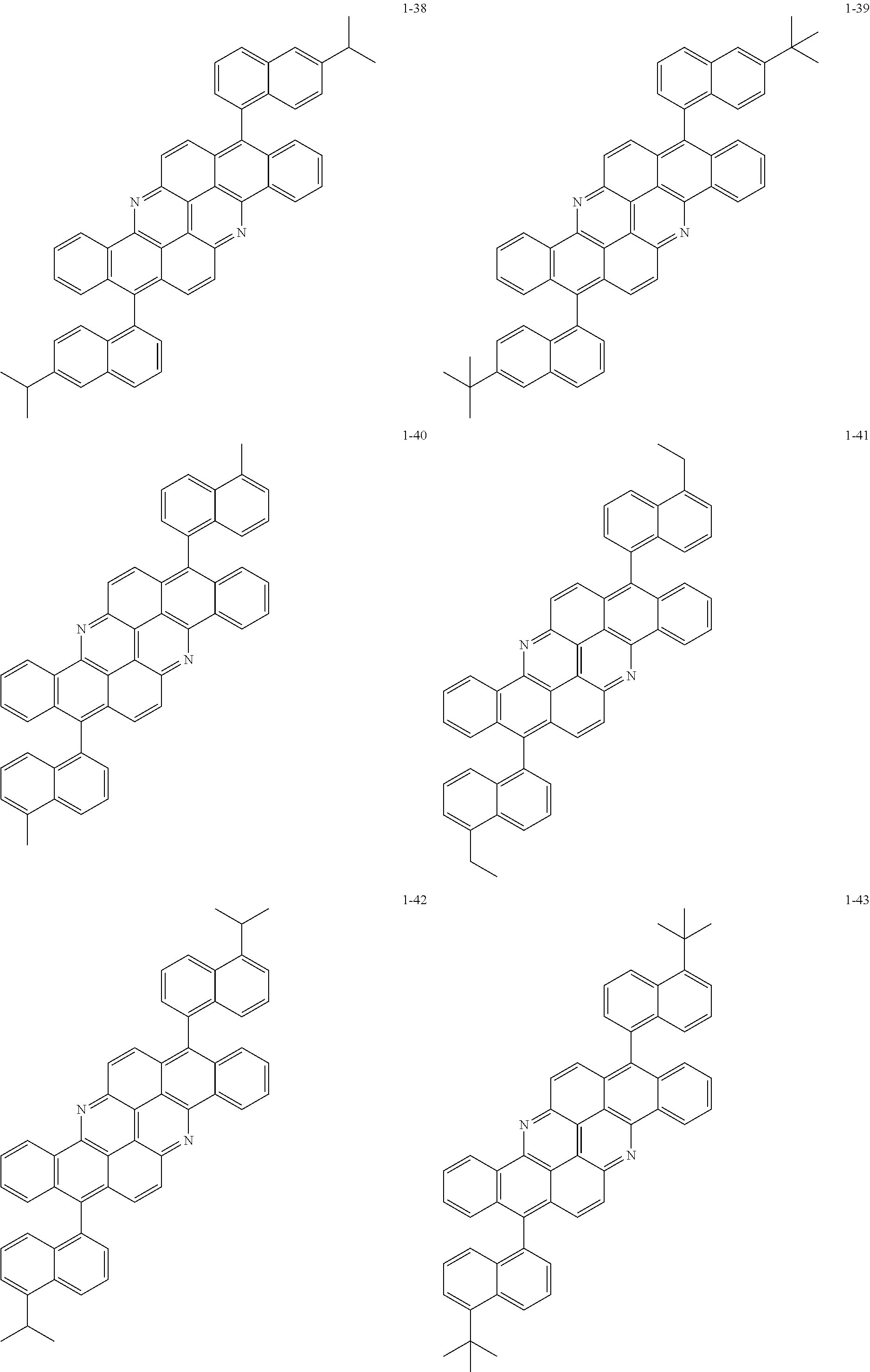

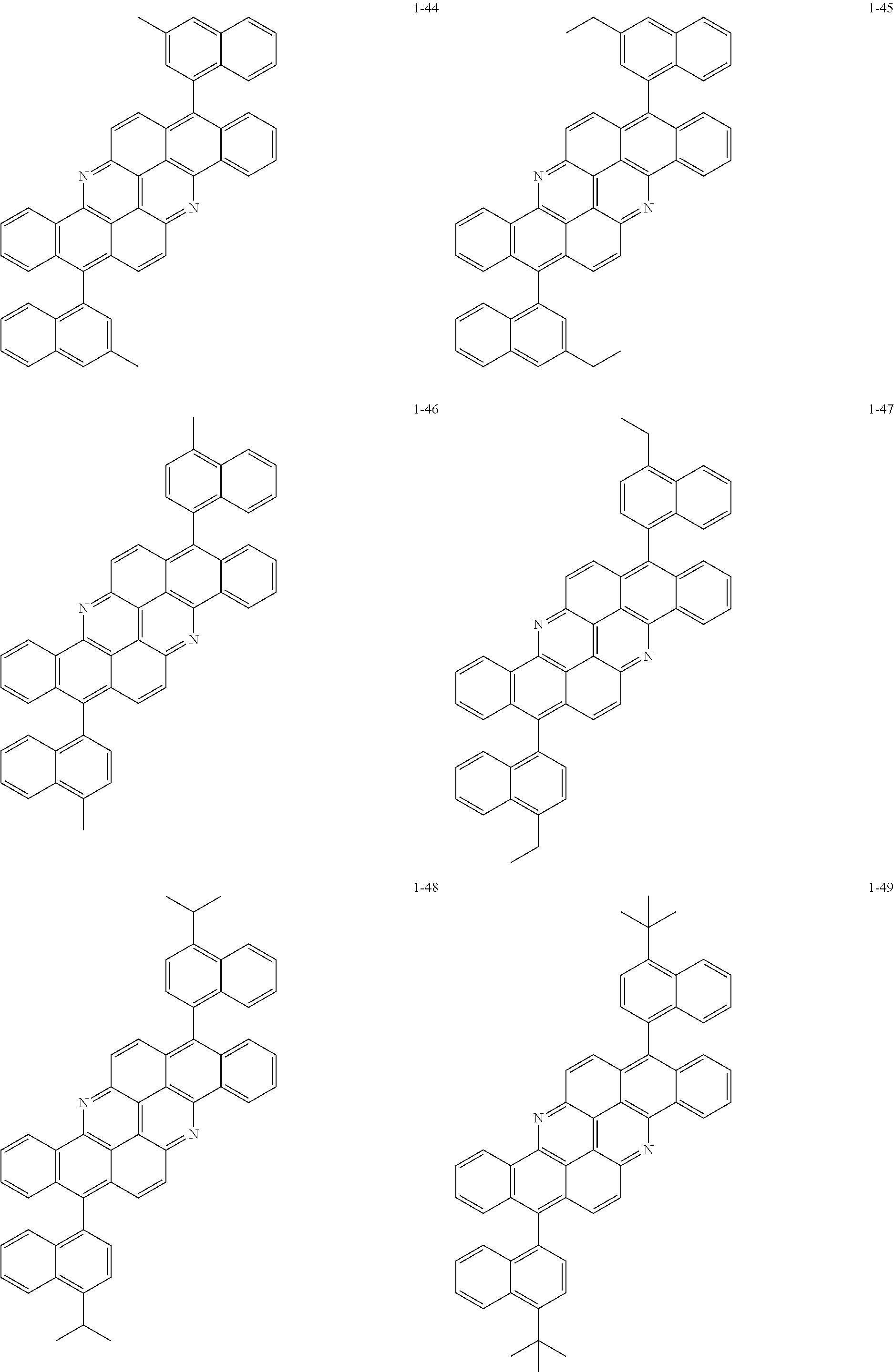

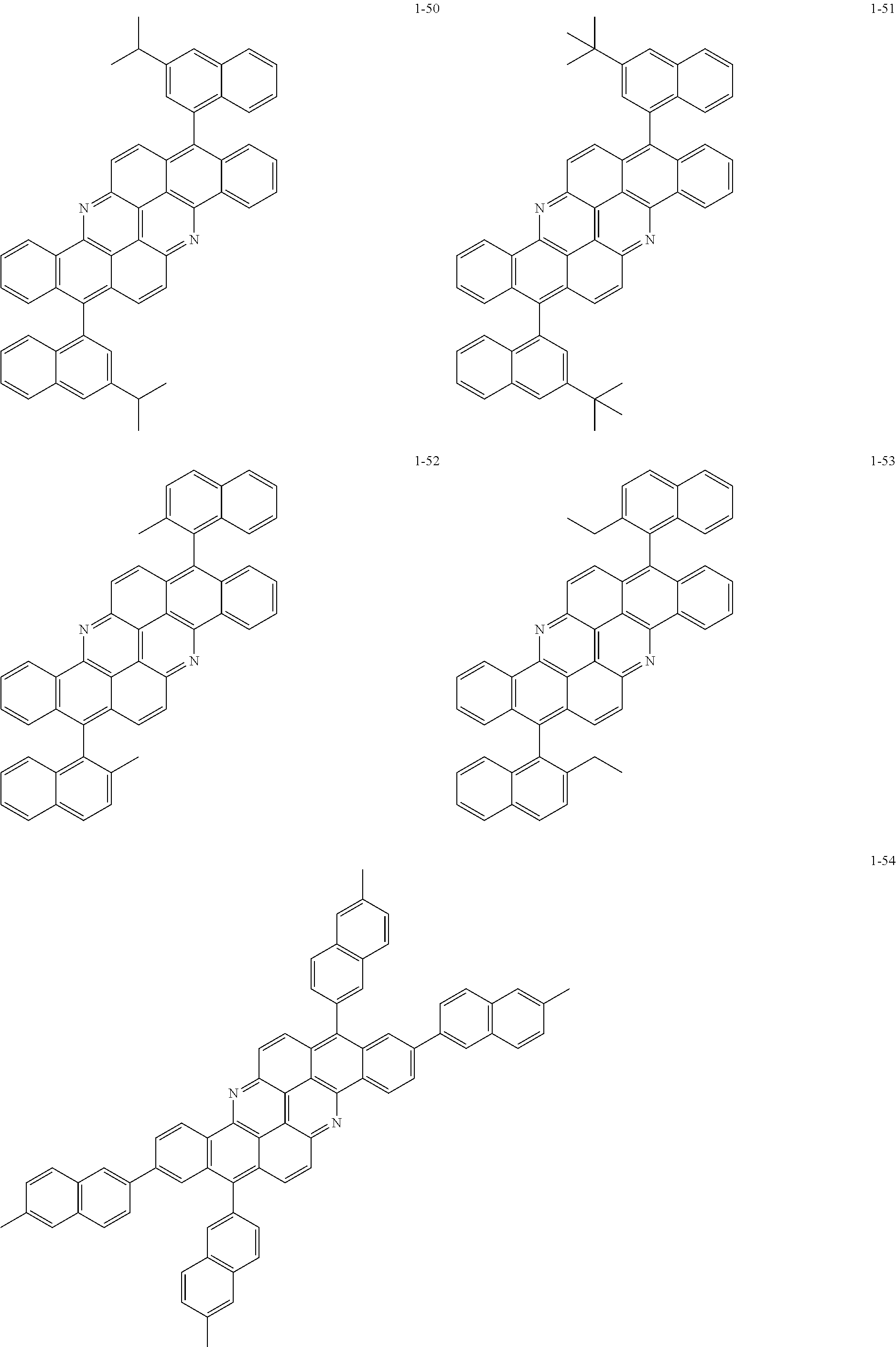

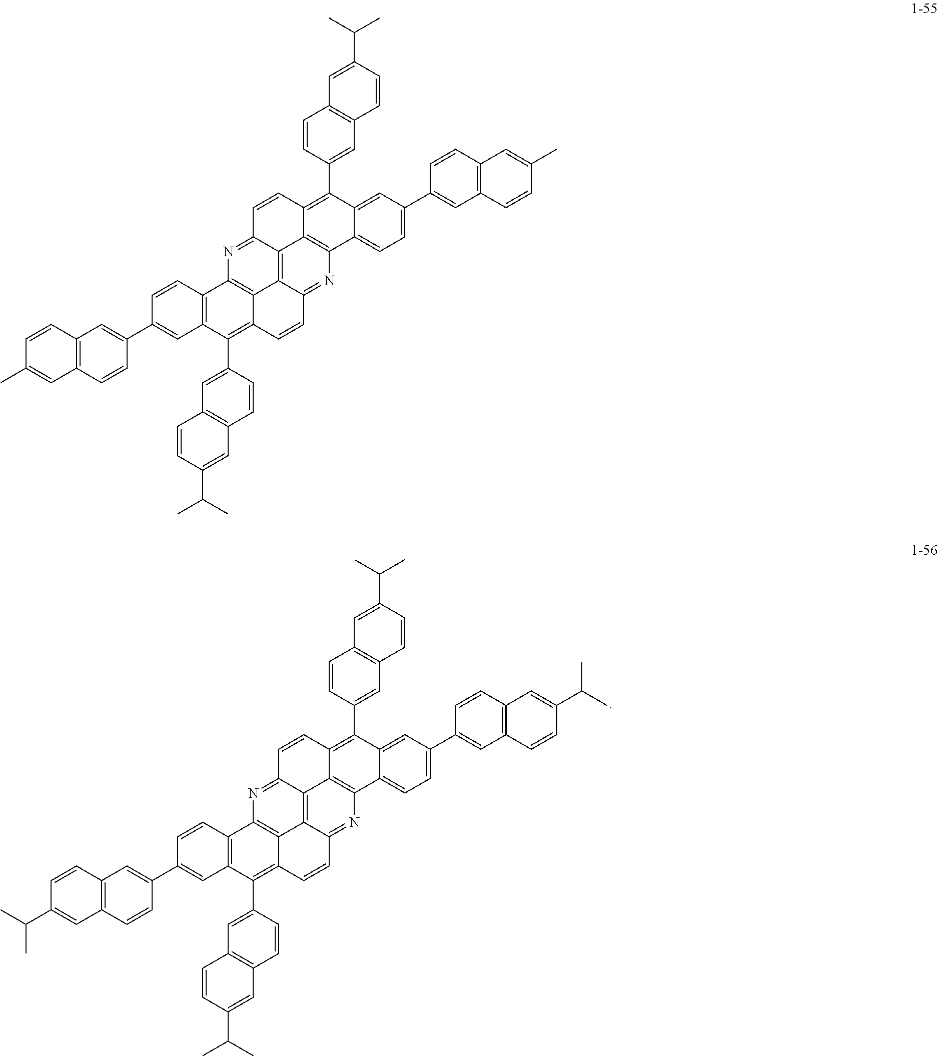

8. An organic light emitting diode, comprising:
a first electrode;
a second electrode facing the first electrode; and
an emissive layer disposed between the first electrode and the second electrode,
wherein the emissive layer comprises an organic compound of Chemical Formula 1: 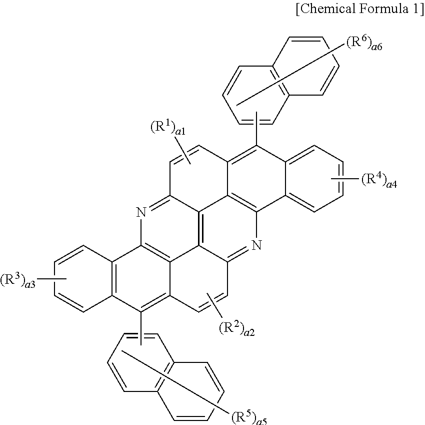
wherein, in Chemical Formula 1,
each of R¹, R², R³, R⁴, R⁵ and R⁶ is independently a halogen atom, a cyano group, an unsubstituted or substituted C₁-C₂₀ alkyl group, an unsubstituted or substituted C₁-C₂₀ alkyl amino group, an unsubstituted or substituted C₆-C₃₀ aryl group, an unsubstituted or substituted C₃-C₃₀ hetero aryl group, an unsubstituted or substituted C₆-C₃₀ aryl amino group or an unsubstituted or substituted C₃-C₃₀ hetero aryl amino group, where each R¹ is identical to or different from each other when a1 is 2, each R² is identical to or different from each other when a2 is 2, each R³ is identical to or different from each other when a3 is 2, 3 or 4, each R⁴ is identical to or different from each other when a4 is 2, 3 or 4, each R⁵ is identical to or different from each other when a5 is 2, 3, 4, 5, 6 or 7 and each R⁶ is identical to or different from each other when a6 is 2, 3, 4, 5, 6 or 7;
each of a1 and a2 is independently an integer of 0, 1 or 2;
each of a3 and a4 is independently an integer of 0, 1, 2, 3 or 4; and
each of a5 and a6 is independently an integer of 0, 1, 2, 3, 4, 5, 6 or 7.

9. The organic light emitting diode of claim 8, wherein the organic compound has a structure of Chemical Formula 2A or Chemical Formula 2B: 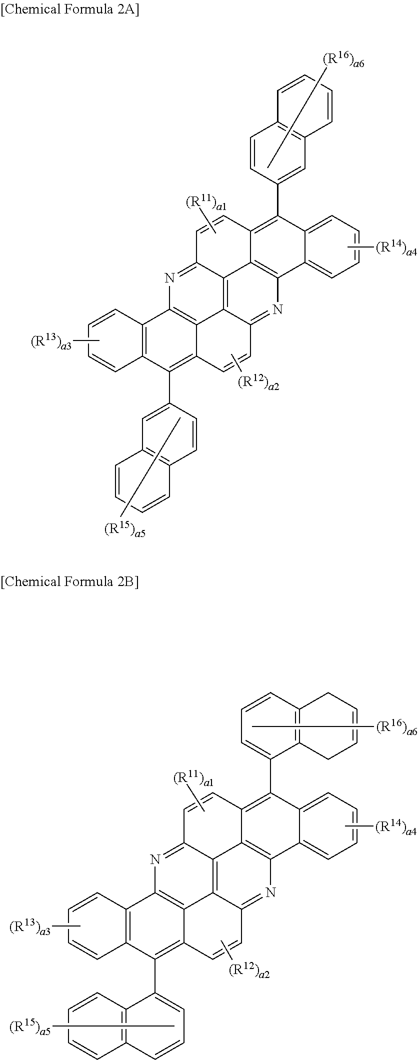
wherein, in Chemical Formulae 2A and 2B,
each of a1, a2, a3, a4, a5 and a6 is the same as defined in Chemical Formula 1,
each of R¹¹, R¹², R¹³, R¹⁴, R¹⁵ and R¹⁶ is independently an unsubstituted or substituted C₁-C₂₀ alkyl group or an unsubstituted or C₁-C₁₀ alkyl-substituted C₆-C₃₀ aryl group, where each R¹¹ is identical to or different from each other when a1 is 2, each R¹² is identical to or different from each other when a2 is 2, each R¹³ is identical to or different from each other when a3 is 2, 3 or 4, each R¹⁴ is identical to or different from each other when a4 is 2, 3 or 4, each R¹⁵ is identical to or different from each other when a5 is 2, 3, 4, 5, 6 or 7 and each R¹⁶ is identical to or different from each other when a6 is 2, 3, 4, 5, 6 or 7.

10. The organic light emitting diode of claim 8, wherein each of R³, R⁴, R⁵ and R⁶ is independently a unsubstituted or substituted C₁-C₁₀ alkyl group or an unsubstituted or C₁-C₁₀ alkyl-substituted C₆-C₃₀ aryl group, each of a1 and a2 is 0, and each of a3, a4, a5 and a6 is independently 0 or 1.

11. The organic light emitting diode of claim 8, wherein each of a1 and a2 is 0, each of R³ and R⁴ is independently an unsubstituted or C₁-C₁₀ alkyl-substituted C₆-C₃₀ aryl group, each of a3 and a4 is independently 0 or 1, each of R⁵ and R⁶ is independently an unsubstituted or substituted C₁-C₁₀ alkyl group, and each of a5 and a6 is independently 0 or 1.

12. The organic light emitting diode of claim 8, wherein each of a1 to a4 is 0, each of R⁵ and R⁶ is independently an unsubstituted or substituted C₁-C₁₀ alkyl group, and each of a5 and a6 is independently 0 or 1.

13. The organic light emitting diode of claim 9, wherein the organic compound has the structure of Chemical Formula 2A, each of a1 to a4 is 0, each of R¹⁵ and R¹⁶ is independently an unsubstituted or substituted C₁-C₁₀ alkyl group, and each of a5 and a6 is independently 0 or 1.

14. The organic light emitting diode of claim 8, wherein the emissive layer comprises at least one emitting material layer including the organic compound of Chemical Formula 1.

15. The organic light emitting diode of claim 14, wherein the at least one emitting material layer comprises an emitter including the organic compound of Chemical Formula 1.

16. The organic light emitting diode of claim 15, wherein the at least one emitting material layer further comprises a first host.

17. The organic light emitting diode of claim 16, wherein the first host comprises a compound of Chemical Formula 4: 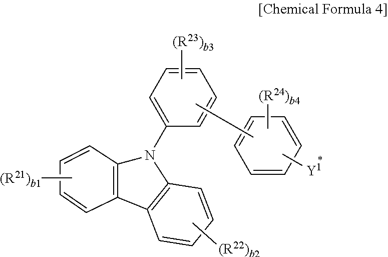
wherein, in Chemical Formula 4,
each of R²¹ and R²² is independently an unsubstituted or substituted C₁-C₂₀ alkyl group or an unsubstituted or substituted C₆-C₃₀ aryl group, where each R²¹ is identical to or different from each other when b1 is 2, 3 or 4, and each R²² is identical to or different from each other when b2 is 2, 3 or 4;
each of R²³ and R²⁴ is independently an unsubstituted or substituted C₁-C₂₀ alkyl group or an unsubstituted or substituted C₆-C₃₀ aryl group, where each R²³ is identical to or different from each other when b3 is 2, 3 or 4, and each R²⁴ is identical to or different from each other when b4 is 2, 3 or 4, or optionally,
with proviso that R²³ and R²⁴ are optionally linked together to form an unsubstituted or substituted hetero ring;
Y¹ has a structure of Chemical Formula 5A or Chemical Formula 5B;
each of b1, b2, b3 and b4 is independently an integer of 0, 1, 2, 3 or 4; and
an asterisk indicates a link to Chemical Formula 5A or Chemical Formula 5B: 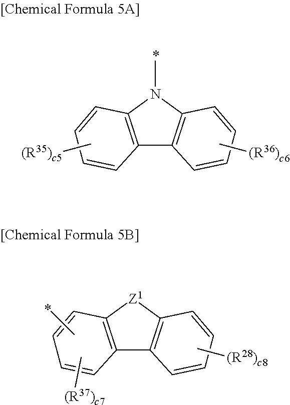
wherein, in Chemical Formulae 5A and 5B,
each of R³⁵, R³⁶, R³⁷ and R³⁸ is independently an unsubstituted or substituted C₁-C₂₀ alkyl group or an unsubstituted or substituted C₆-C₃₀ aryl group, where each R³⁵ is identical to or different from each other when c5 is 2, 3 or 4, each R³⁶ is identical to or different from each other when c6 is 2, 3 or 4, each R³⁷ is identical to or different from each other when c7 is 2 or 3, and each R³⁸ is identical to or different from each other when c8 is 2, 3 or 4;
Z¹ is NR³⁹, O or S, where R³⁹ is hydrogen, an unsubstituted or substituted C₁-C₂₀ alkyl group or an unsubstituted or substituted C₆-C₃₀ aryl group;
each of c5, c6 and c8 is independently an integer of 0, 1, 2, 3 or 4;
c7 is an integer of 0, 1, 2 or 3; and
an asterisk indicates a link position.

18. The organic light emitting diode of claim 17, wherein the first host is at least one of the following compounds 2-1 to 2-24:

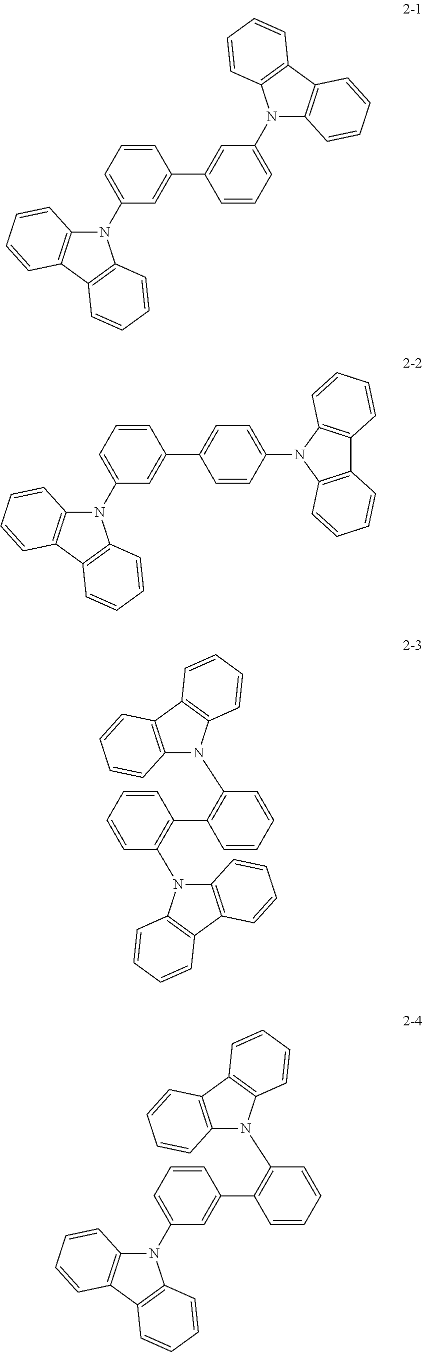 [SMILES: c1cc(-c2cccc(-n3c4ccccc4c4ccccc43)c2)cc(-n2c3ccccc3c3ccccc32)c1 ; c1cc(-c2ccc(-n3c4ccccc4c4ccccc43)cc2)cc(-n2c3ccccc3c3ccccc32)c1 ; c1ccc(-n2c3ccccc3c3ccccc32)c(-c2ccccc2-n2c3ccccc3c3ccccc32)c1 ; c1cc(-c2ccccc2-n2c3ccccc3c3ccccc32)cc(-n2c3ccccc3c3ccccc32)c1]

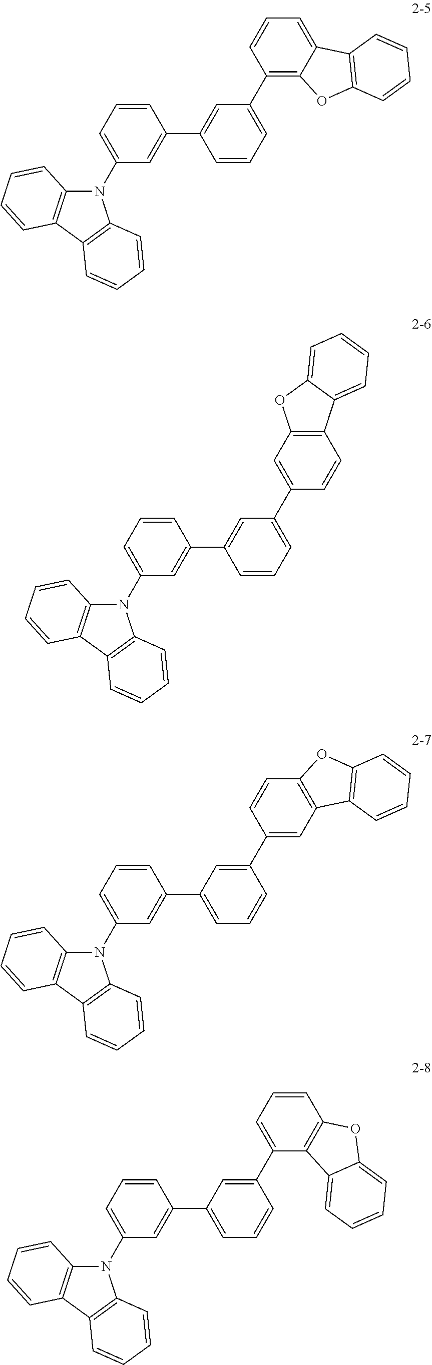 [SMILES: c1cc(-c2cccc(-n3c4ccccc4c4ccccc43)c2)cc(-c2cccc3c2oc2ccccc23)c1 ; c1cc(-c2cccc(-n3c4ccccc4c4ccccc43)c2)cc(-c2ccc3c(c2)oc2ccccc23)c1 ; c1cc(-c2cccc(-n3c4ccccc4c4ccccc43)c2)cc(-c2ccc3oc4ccccc4c3c2)c1 ; c1cc(-c2cccc(-n3c4ccccc4c4ccccc43)c2)cc(-c2cccc3oc4ccccc4c23)c1]

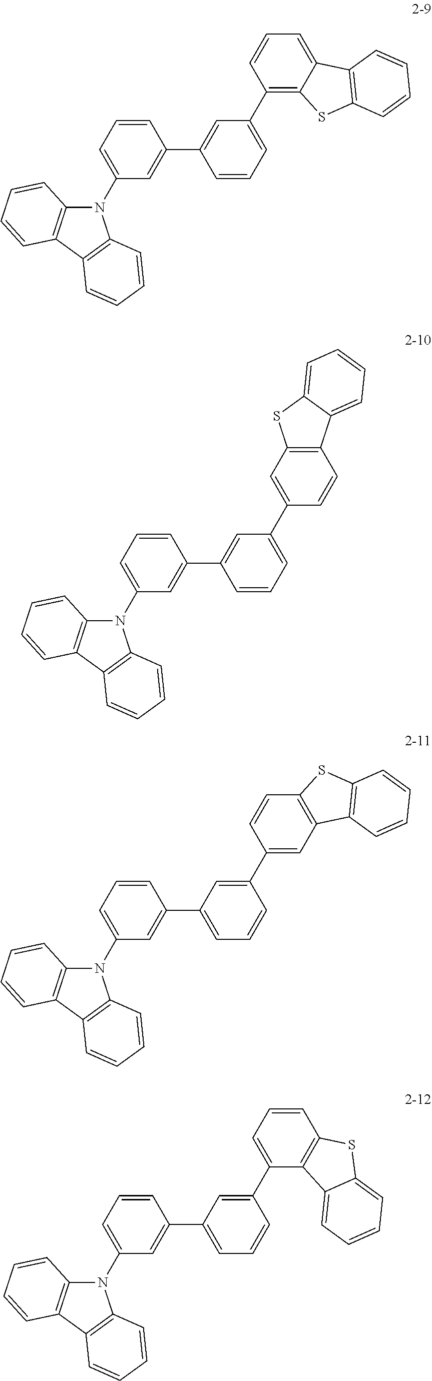 [SMILES: c1cc(-c2cccc(-n3c4ccccc4c4ccccc43)c2)cc(-c2cccc3c2sc2ccccc23)c1 ; c1cc(-c2cccc(-n3c4ccccc4c4ccccc43)c2)cc(-c2ccc3c(c2)sc2ccccc23)c1 ; c1cc(-c2cccc(-n3c4ccccc4c4ccccc43)c2)cc(-c2ccc3sc4ccccc4c3c2)c1 ; c1cc(-c2cccc(-n3c4ccccc4c4ccccc43)c2)cc(-c2cccc3sc4ccccc4c23)c1]

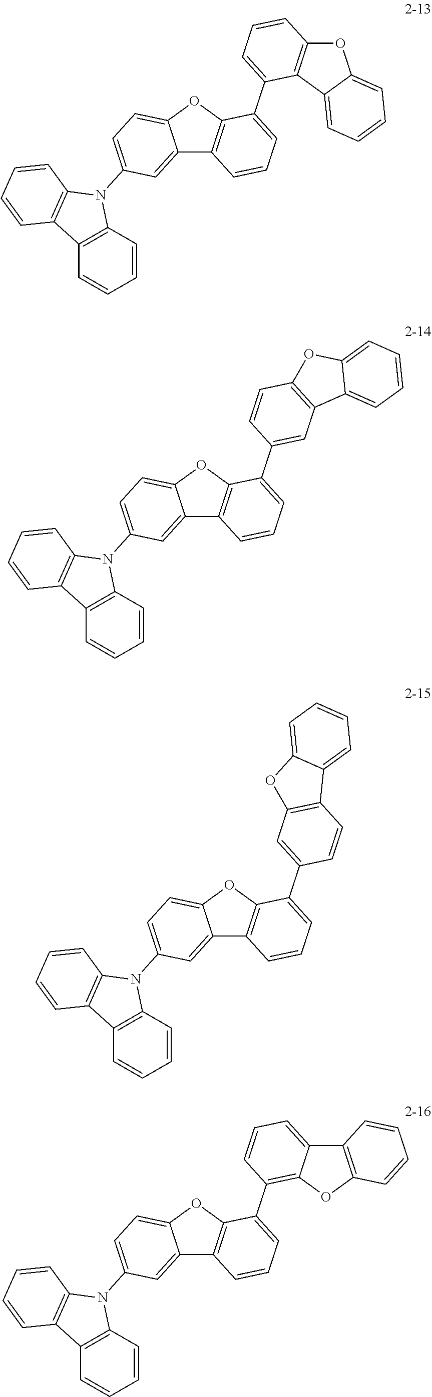 [SMILES: c1ccc2c(c1)oc1cccc(-c3cccc4c3oc3ccc(-n5c6ccccc6c6ccccc65)cc34)c12 ; c1ccc2c(c1)oc1ccc(-c3cccc4c3oc3ccc(-n5c6ccccc6c6ccccc65)cc34)cc12 ; c1ccc2c(c1)oc1cc(-c3cccc4c3oc3ccc(-n5c6ccccc6c6ccccc65)cc34)ccc12 ; c1ccc2c(c1)oc1c(-c3cccc4c3oc3ccc(-n5c6ccccc6c6ccccc65)cc34)cccc12]

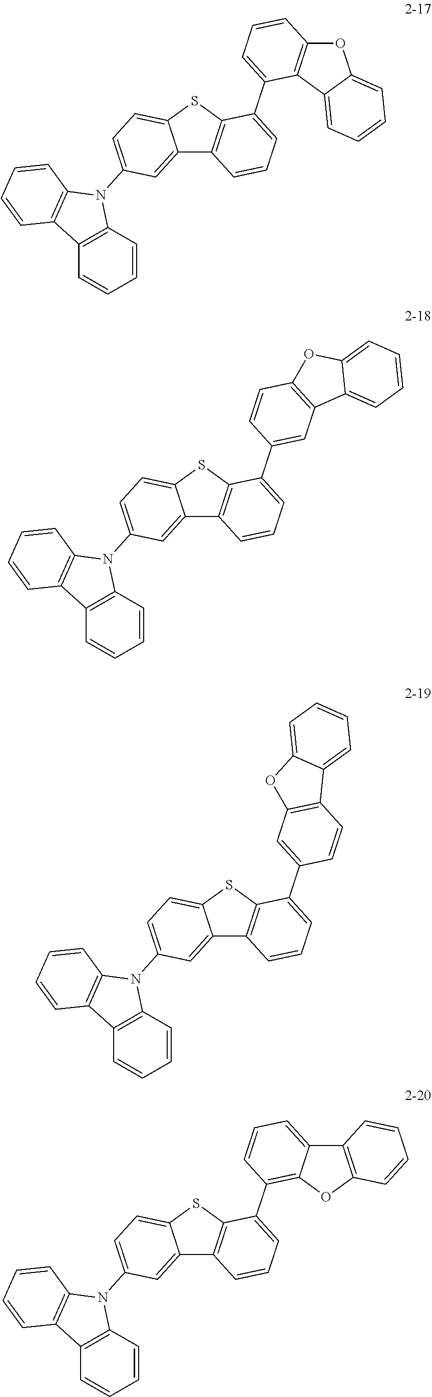 [SMILES: c1ccc2c(c1)oc1cccc(-c3cccc4c3sc3ccc(-n5c6ccccc6c6ccccc65)cc34)c12 ; c1ccc2c(c1)oc1ccc(-c3cccc4c3sc3ccc(-n5c6ccccc6c6ccccc65)cc34)cc12 ; c1ccc2c(c1)oc1cc(-c3cccc4c3sc3ccc(-n5c6ccccc6c6ccccc65)cc34)ccc12 ; c1ccc2c(c1)oc1c(-c3cccc4c3sc3ccc(-n5c6ccccc6c6ccccc65)cc34)cccc12]

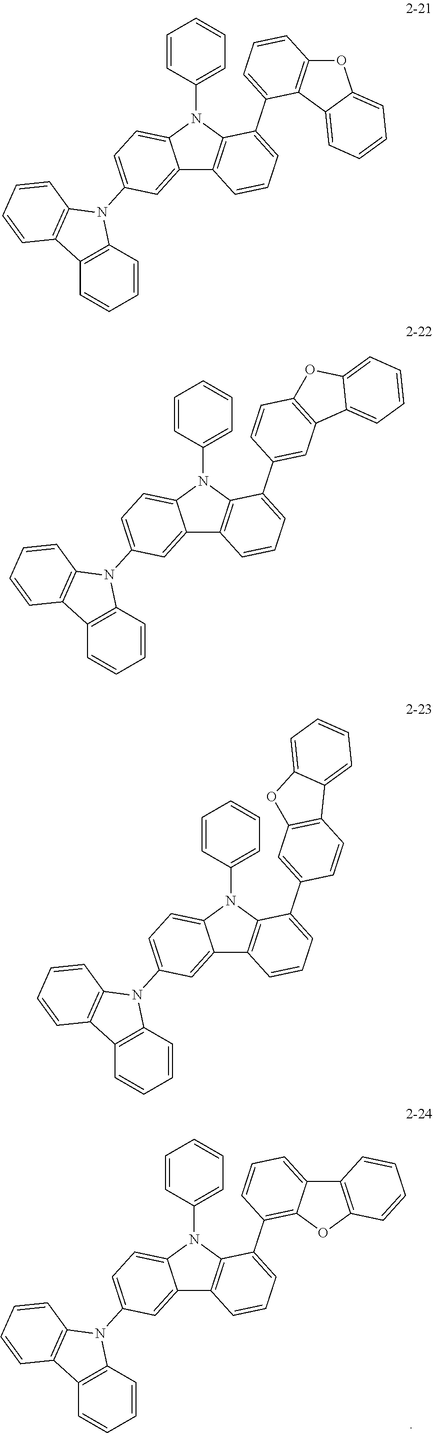 [SMILES: c1ccc(-n2c3ccc(-n4c5ccccc5c5ccccc54)cc3c3cccc(-c4cccc5oc6ccccc6c45)c32)cc1 ; c1ccc(-n2c3ccc(-n4c5ccccc5c5ccccc54)cc3c3cccc(-c4ccc5oc6ccccc6c5c4)c32)cc1 ; c1ccc(-n2c3ccc(-n4c5ccccc5c5ccccc54)cc3c3cccc(-c4ccc5c(c4)oc4ccccc45)c32)cc1 ; c1ccc(-n2c3ccc(-n4c5ccccc5c5ccccc54)cc3c3cccc(-c4cccc5c4oc4ccccc45)c32)cc1]

19. The organic light emitting diode of claim 16, wherein the at least one emitting material layer further comprises a second host.

20. The organic light emitting diode of claim 8, wherein the emissive layer has a single emitting part.

21. The organic light emitting diode of claim 8, wherein the emissive layer comprises:
a first emitting part disposed between the first and second electrodes and including a first emitting material layer;
a second emitting part disposed between the first emitting part and the second electrode and including a second emitting material layer; and
a first charge generation layer disposed between the first emitting part and the second emitting part, and
wherein at least one of the first emitting material layer or the second emitting material layer comprises the organic compound of Chemical Formula 1.

22. The organic light emitting diode of claim 21, wherein the second emitting material layer comprises:
a first layer disposed between the first charge generation layer and the second electrode; and
a second layer disposed between the first layer and the second electrode;
wherein one of the first layer or the second layer comprises the organic compound.

23. The organic light emitting diode of claim 21, wherein the emissive layer further comprises:
a third emitting part disposed between the second emitting part and the second electrode and including a third emitting material layer; and
a second charge generation layer disposed between the second emitting part and the third emitting part,
wherein the second emitting material layer comprises:
a first layer disposed between the first charge generation layer and the second charge generation layer; and
a second layer disposed between the first layer and the second charge generation layer,
wherein one of the first layer or the second layer comprises the organic compound of Chemical Formula 1.

24. An organic light emitting device, comprising:
a substrate; and
the organic light emitting diode according to claim 8 on the substrate.
